(19) United States
(12) Patent Application Publication
(10) Pub. No.: US 2003/0143656 A1
(43) Pub. Date: Jul. 31, 2003

(54) Protein kinase regulation

(76) Inventors: Dario Alessi, Dundee (GB); Ricardo Biondi, Dundee (GB)

Correspondence Address: Karla M Weyand, Braman & Rogalskyj, PO Box 352, Canadaigua, NY, 14424-0352, US

(21) Appl. No.: 10/148,786

(22) PCT Filed: Dec. 4, 2000
(86) PCT No.: PCT/GB00/04598

(51) Int. Cl. C12Q 1/48; C12N 9/12

(52) U.S. Cl. 435/15; 435/194

(57) ABSTRACT

A method of identifying a compound that modulates the protein kinase activity of a protein kinase having a hydrophobic pocket in the position equivalent to the hydrophobic pocket of Protein Kinase A (PKA) that is defined by residues including Lys76, Leu116, Val80 and/or Lys111 of full-length mouse PKA, wherein the ability of the compound to inhibit, promote or mimic the interaction of the said hydrophobic pocket-containing protein kinase with an interacting polypeptide is measured and a compound that inhibits, promotes or mimics the said interaction interacts is selected, wherein the interacting polypeptide with the hydrophobic pocket of the protein kinase and/or comprises the amino acid sequence Phe/Tyr-Xaa-Xaa-Phe/Tyr.

The present invention relates to regulation of protein kinases.

Stimulation of cells with insulin and growth factors generates the second messengers PtdIns (3, 4, 5,) P3 and PtdIns (3, 4) P₂ (Leevers et al (1999) Curr Opin Biol 11, 219-225) which induce the activation of certain members of the AGC subfamily of protein kinases that include protein kinase B (PKB) (Shepherd et al (1998) Biochem J 333: 471-479; Alessi & Downes (1998) Biochem Biophys Acta 1436, 151-164), p70 S6 kinase (S6K) (Proud C G (1995) Trends in Biochem Sci 21, 181-185; Pullen & Thomas (1998) FEBS LETT 410, 78-82), serum and glucocorticoid-induced kinase (SGK) (Kobayashi & Cohen (1999) Biochem J 339, 319-328; Park et al (1999) EMBO J 18, 3024-3033) and protein kinase C (PKC) isoforms (Mellor & Parker (1998) Biochem J, 332, 281-292). These kinases can then mediate many of the effects of insulin and growth factors by phosphorylating key regulatory proteins (reviewed in Shepherd et al (1998) Biochem J 333, 471-479; Alessi & Downes (1998) Biochem Biophys Acta 1436, 151-164 and Alessi & Cohen (1998) Curr Opin Genet Dev 8, 55-62).

The interaction of PtdIns (3, 4, 5) P₃ with the PH domain of PKB causes PKB to translocate to the plasma membrane where it is activated by phosphorylation of two residues, namely Thr308 and Ser473. Both of these residues need to be phosphorylated for maximal activation and their phosphorylation in vivo is prevented by inhibitors of phosphatidylinositol (PI) 3-kinase (Shepherd et al (1998); Alessi & Downes (1998)). Thr308 lies in the activation loop of the kinase domain while Ser473 is located C-terminal to the catalytic domain, in a region that displays high homology between different AGC family members. Importantly, p70 S6K (Pearson et al (1995) EMBO J 14, 5278-5287), PKC isoforms (Mellor & Parker (1998)) and SGK (Kobayashi & Cohen (1999); Park et al (1999)) also possess residues lying in sequences equivalent to Thr308 and Ser473 of PKB, whose phosphorylation is necessary for activation of these kinases in vivo. Ser473 of PKB and the equivalent residues of p70 S6 kinase, PKC and SGK lie in a hydrophobic motif: Phe-Xaa-Xaa-Phe-Ser/Thr-Phe/Tyr distinct from the sequences surrounding Thr308.

The protein kinase termed 3-phosphoinositide-dependent protein kinase-1 (PDK1) plays a central role in activating AGC subfamily members (reviewed in Belham et al (1999) Current Biol 9, R93-R96; Peterson & Schreiber (1999) Current Biol 9, R521-524]). PDK1 phosphorylates PKB at Thr308 (Alessi et al (1997) Curr Biol 7, 261-269; Alessi et al (1997) Curr Biol 7, 776-789; Stokoe et al (1997) Science 277, 567-570; Stephens et al (1998) Science 279, 710-714) and the equivalent residues on PKC isoforms (LeGood et al (1998) Science, 281, 2042-2045; Chou et al (1998) Curr Biol 8, 1069-1077; Dutil et al (1998) Curr Biol 8, 1366-1375), p70 S6 kinase (Alessi et al (1998) Curr Biol 8, 69-81; Pullen et al (1998) Science, 279, 707-710) and SGK (Kobayashi & Cohen (1999); Park et al (1999)). Cyclic AMP-dependent protein kinase (PKA) is also phosphorylated by PDK1 at the equivalent residue (Thr197) and this is required for PKA activity (Chen et al (1998) Proc Natl Acad Sci, USA 95, 9849-9854). However, unlike the other members of the AGC subfamily of protein kinases discussed above, PKA does not possess a residue equivalent to Ser473 of PKB. Instead, its amino acid sequence terminates with the sequence Phe-Xaa-Xaa-PheCOOH corresponding to the first part of the hydrophobic motif Phe-Xaa-Xaa-Phe-Ser/Thr-Phe/Tyr that surrounds Ser473 (the “PDK2” phosphorylation motif). Nevertheless, this C-terminal region of PKA plays an important role as its mutation or deletion greatly diminishes activity (Etchebehere et al (1997) Eur J Biochem, 248, 820-826).

Recently, we discovered that the kinase domain of PDK1 interacts with a region of Protein kinase C-Related Kinase-2 (PRK2) termed the PDK1 Intracting Fragment (PIF). This converts PDK1 from a form that phosphorylates PKB at Thr308 to a form that phosphorylates PKB at both Thr308 and Ser473 (Balendran et at (1999) Current Biology 9, 393-404). PIF contains a hydrophobic sequence motif (Phe-Xaa-Xaa-Phe-Asp-Tyr) similar to that found in PKB, except that the residue equivalent to Ser473 is an Asp. Mutation of any of the conserved aromatic residues in this motif or mutation of the Asp residue to either Ala or Ser greatly weakens the interaction of PIF with PDK1, indicating that PIF-binds to PDK1 via these residues (Balendran et at (1999).

p70 S6 kinase (p70 S6K or S6K) is activated by insulin and growth factors and mediates the phosphorylation of the 40S ribosomal protein S6 (Proud (1995). Trends in Bioch. Sci 21, 181-185). This enables efficient translation of mRNA molecules containing a polypyrimidine tract at their 5′ transcriptional start sites (Lane et at (1993) Nature 363,170-172). p70 S6K also phosphorylates unknown proteins to permit progression through the G1 phase of the cell cycle (Jefferies et at (1997) EMBO J. 16, 3693-3704).

p70 S6K is activated by insulin and growth factors, through a PI3-kinase dependent pathway, and becomes phosphorylated on at least 7 Ser/Thr residues in response to these agonists. The phosphorylation of two of these residues namely Thr252 and Thr412 on the longer splice variant of the α-isoform (Thr229 and Thr389 on the shorter splice variant) appear to make the most important contribution to the activation of p70 S6K (Pearson et al (1995) EMBO J. 14, 5278-5287; Pullen & Thomas (1998) FEBS LETT.410, 78-82; Weng et al (1998) J. Biol. Chem.273, 16621-16629). Phosphorylation of Thr252 alone or mutation of Thr412 to glutamic acid to mimic phosphorylation of this residue, results in a small activation of p70 S6K. In contrast, phosphorylation of both residues or phosphorylation of Thr252 in the T412E mutant of p70 S6K results in large activation of expressed p70 S6K, showing that phosphorylation of Thr252 and Thr412 leads to a synergistic activation p70 S6K (Weng et al (1998) J. Biol. Chem.273, 16621-16629; Alessi et al (1998) Curr. Biol. 8, 69-81).

The residues surrounding Thr252 and Thr412 of p70 S6K are highly conserved in all AGC family members and phosphorylation of the residues equivalent to Thr252 and Thr412 of p70 S6K is necessary for activation and/or stability of these kinases in vivo (Belham et al (1999) Current Biol. 9, R93-R96), as discussed above. Thr412 is located C-terminal to the catalytic domain, and the residues surrounding Thr412 lie in a Phe-Xaa-Xaa-Phe-Ser/Thr-Phe/Tyr consensus motif.

As discussed above, 3-phosphoinositide dependent protein kinase-1 (PDK1) can phosphorylate p70 S6K at Thr252 in vitro and in transfection experiments. Phosphorylation of p70 S6K by PDK1 in vitro is independent of the presence of PtdIns(3,4,5) P₃, and activation is increased greatly if the non catalytic carboxy terminal tail of p70 S6K is deleted and if Thr412 is mutated to an acidic residue

Recently, we made the surprising observation that PDK1 can be converted from a form that phosphorylates Thr308 of PKB alone (the residue equivalent to Thr252 in p70 S6K) to a form that phosphorylates both Thr308 and Ser 473 (the residue equivalent to Thr412 in p70 S6K) through interaction with a region of Protein Kinase C-Related Kinase-2(PRK2), termed the PDK1 Interacting Fragment (PIF) (Balendran et al (1999) Curr Biol 9(8), 393-404; GB 9906245.7, filed Mar. 19, 1999).

We identify and characterise a hydrophobic pocket at a position equivalent to a hydrophobic pocket on the small lobe of protein kinase A (PKA) on the small lobe of the kinase domain of protein kinases other than PKA, for example PDK1, and identify polypeptides that interact with the hydrophobic pocket. We identify the effect of such polypeptides on the protein kinase activity and stability of such protein kinases. We identify assays and protein kinase substrates that can be used to identify drugs that activate or inhibit the activity of a protein kinase by interacting with the hydrophobic pocket.

A first aspect of the invention provides a method of identifying a compound that modulates the protein kinase activity of a protein kinase having a hydrophobic pocket in the position equivalent to the hydrophobic pocket of mouse Protein Kinase A (PKA) that is defined by residues including Lys76, Leu116, Val80 and/or Lys111 of full-length mouse PKA, wherein the ability of the compound to inhibit, promote or mimic the interaction of the said hydrophobic pocket-containing protein kinase with an interacting polypeptide is measured and a compound that inhibits, promotes or mimics the said interaction is selected, wherein the interacting polypeptide interacts with the hydrophobic pocket of the protein kinase and/or comprises the amino acid sequence Phe/Tyr-Xaa-Xaa-Phe/Tyr.

The residue immediately C-terminal of the Phe/Tyr-Xaa-Xaa-Phe/Tyr sequence may be any residue. Preferably, the interacting polypeptide comprises the amino acid sequence Phe/Tyr-Xaa-Xaa-Phe/Tyr-Zaa-Phe/Tyr, wherein Zaa represents a negatively charged amino acid residue. Also preferably, the interacting polypeptide may have the C-terminal sequence Phe/Tyr-Xaa-Xaa-Phe/Tyr-COOH, preferably Phe-Xaa-Xaa-Phe-COOH, or Phe/Tyr-Xaa-Xaa-Phe/Tyr-(X)ₙ—COOH, preferably Phe-Xaa-Xaa-Phe-(X)ₙ—COOH, wherein n is between 1 and 20, 15, 10, 5, 4, 3 or 2, preferably between 1 and 4, most preferably 4. Each amino acid X is any amino acid residue, preferably glycine. Thus, it is preferred that the interacting polypeptide has the C-terminal sequence Phe-Xaa-Xaa-Phe-(Gly)₄-COOH. The interacting polypeptide preferably does not have the amino acid sequence Phe/Tyr-Xaa-Xaa-Phe/Tyr-Ser/Thr-Phe/Tyr. When the interacting polypeptide, for example comprising the amino acid sequence Phe/Tyr-Xaa-Xaa-Phe/Tyr is not part of the same polypeptide chain as the protein kinase, it is preferred that the interacting polypeptide has fewer than about 400, 380, 350, 300, 250, 200, 150, 100, 80, 50, 40 or 30 amino acids. When the hydrophobic pocket-containing polypeptide is PDK1, it is preferred that the interacting polypeptide is not full length PKB or SGK (phosphorylated or unphosphorylated forms) or other known naturally occurring substrate of PDK1, for example PKCζ.

The negatively charged amino acid residue Zaa may be, for example, an aspartate, glutamate, phosphorylated serine (phosphoserine), phosphorylated threonine (phosphothreonine) or phosphorylated tyrosine (phosphotyrosine) residue, or a negatively charged non-naturally occuring residue. It is preferred that Zaa is an aspartate, glutamate, phosphoserine or phosphothreonine residue, still more preferably an aspartate or glutamate residue. It is preferred that the first residue in the sequence corresponding to any of the above consensus sequences is a phenylalanine residue. Phenylalanine is found in this position in naturally occuring polypeptides in which a said consensus sequence has been identified. It may also be preferred that the fourth residue in the sequence corresponding to any of the above consensus sequences is a phenylalanine residue. Phenylalanine and tyrosine are both (separately) found in this position in naturally occuring polypeptides in which a said consensus sequence has been identified.

Preferred interacting polypeptides in which the residue immediately C-terminal of the Phe/Tyr-Xaa-Xaa-Phe/Tyr amino acid sequence is not a negatively charged amino acid residue may comprise the amino acid sequence FEGFA or FAGFS.

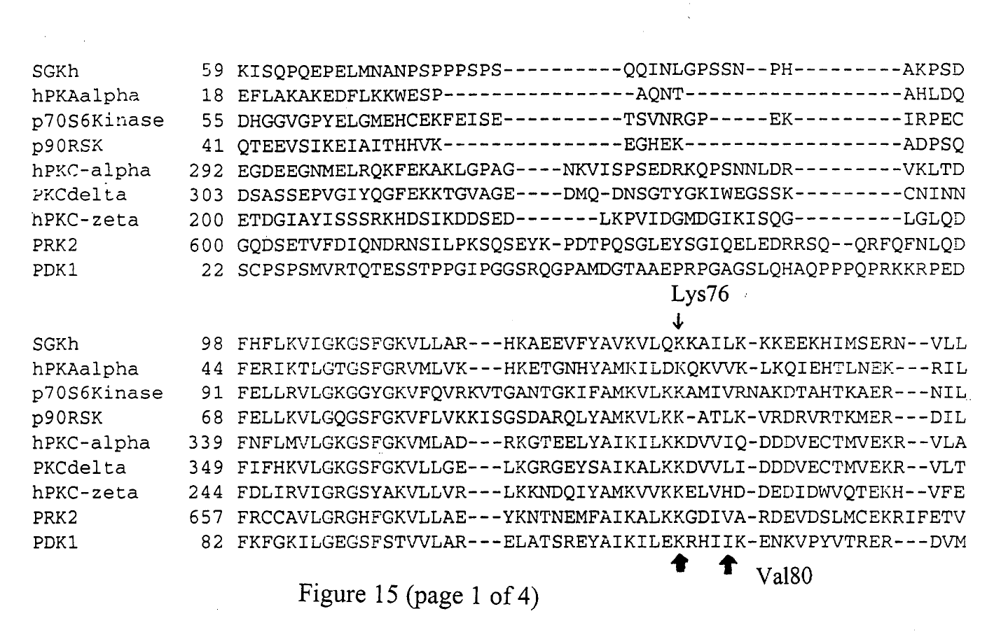

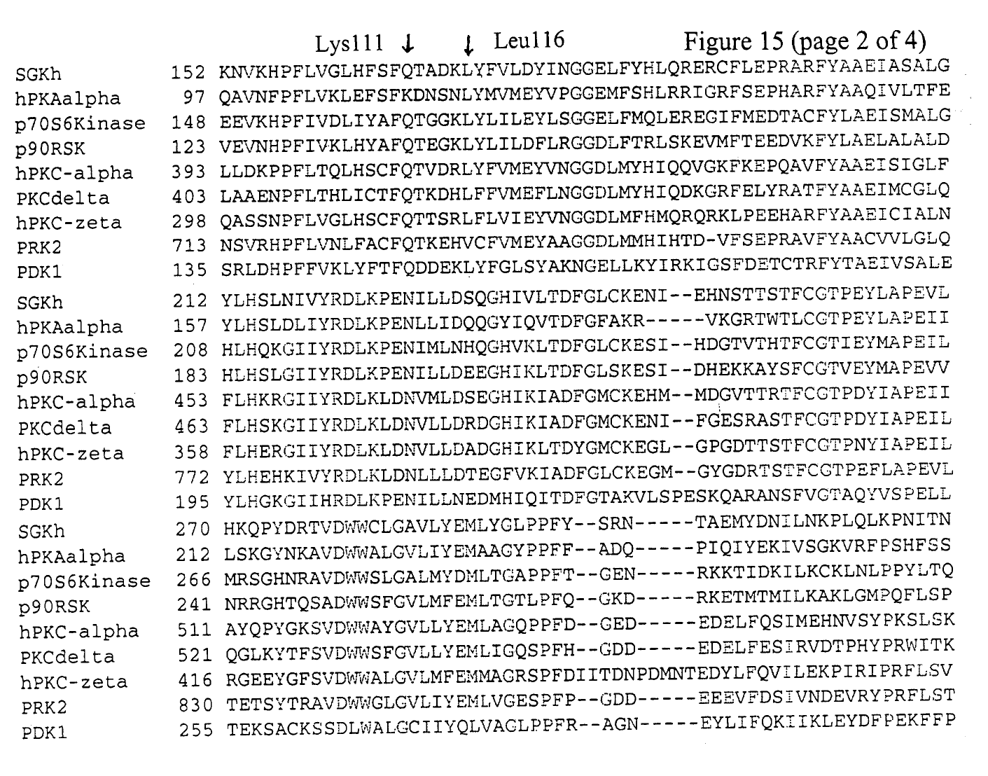

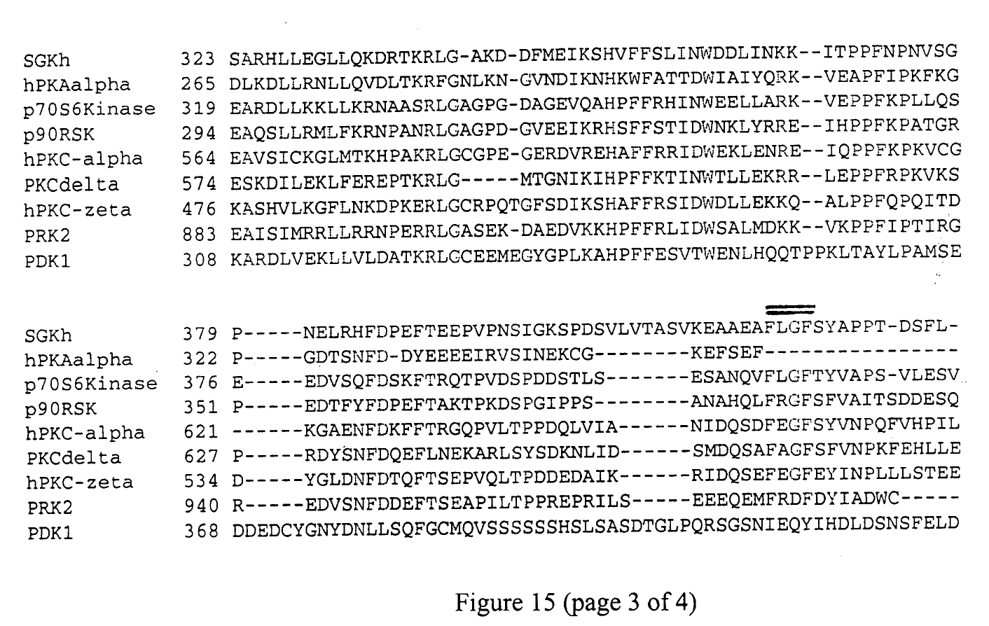

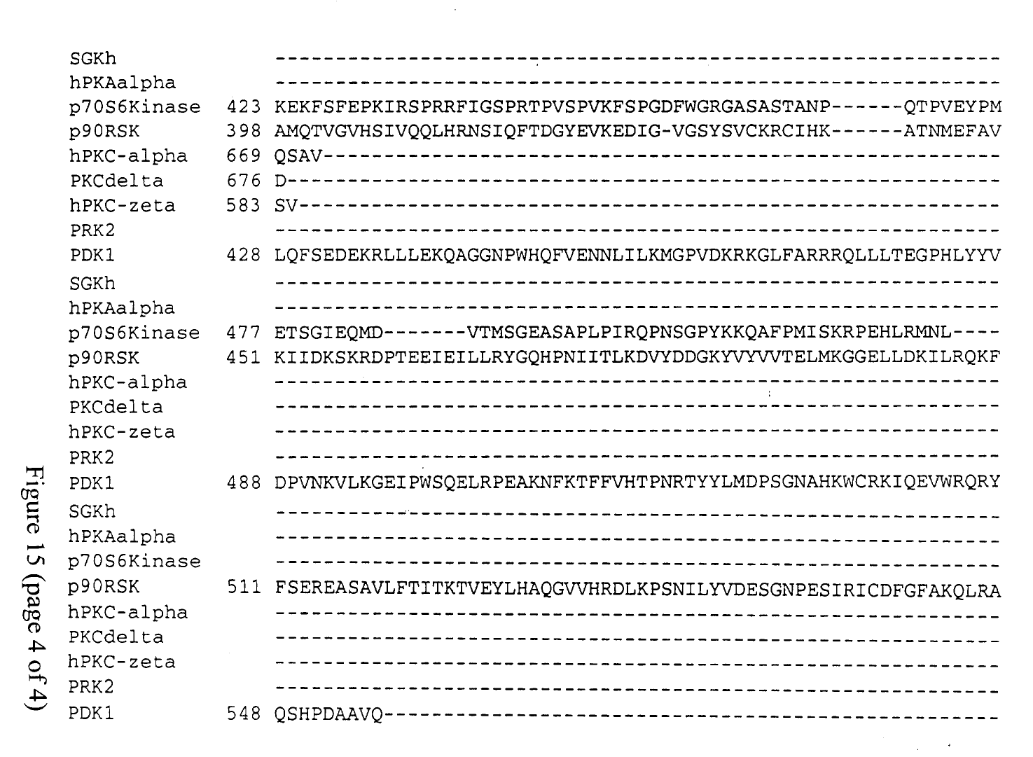

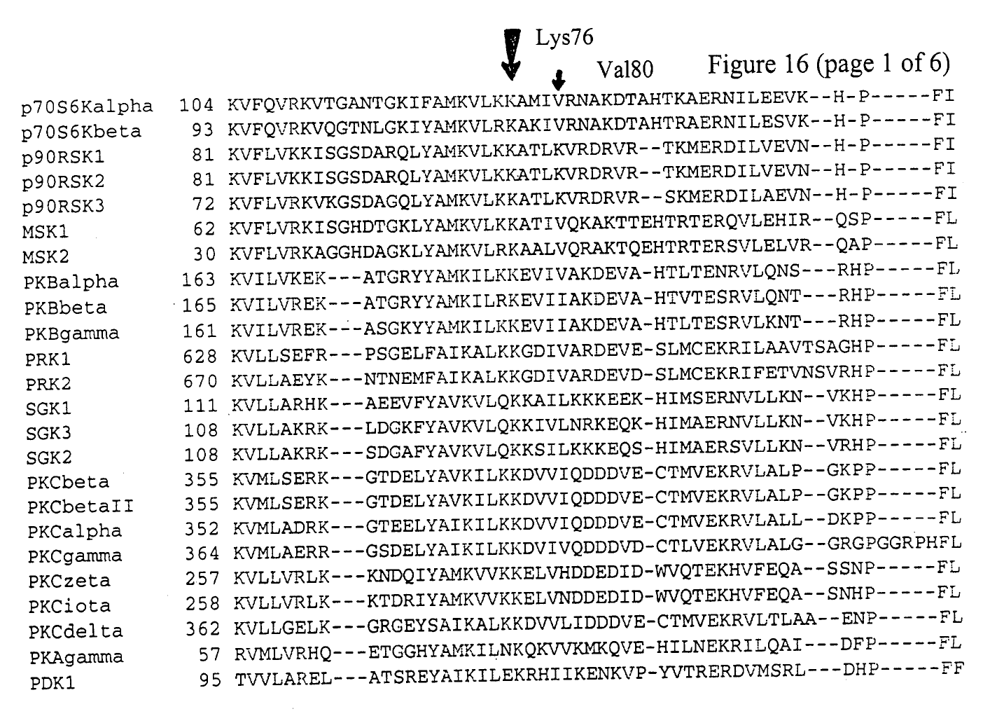

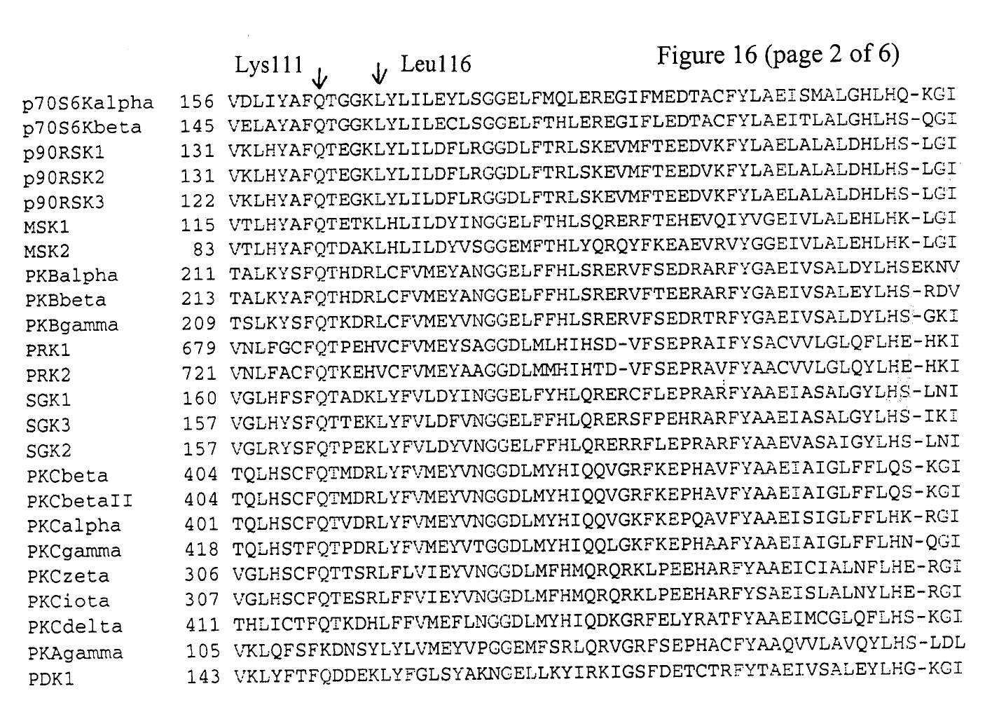

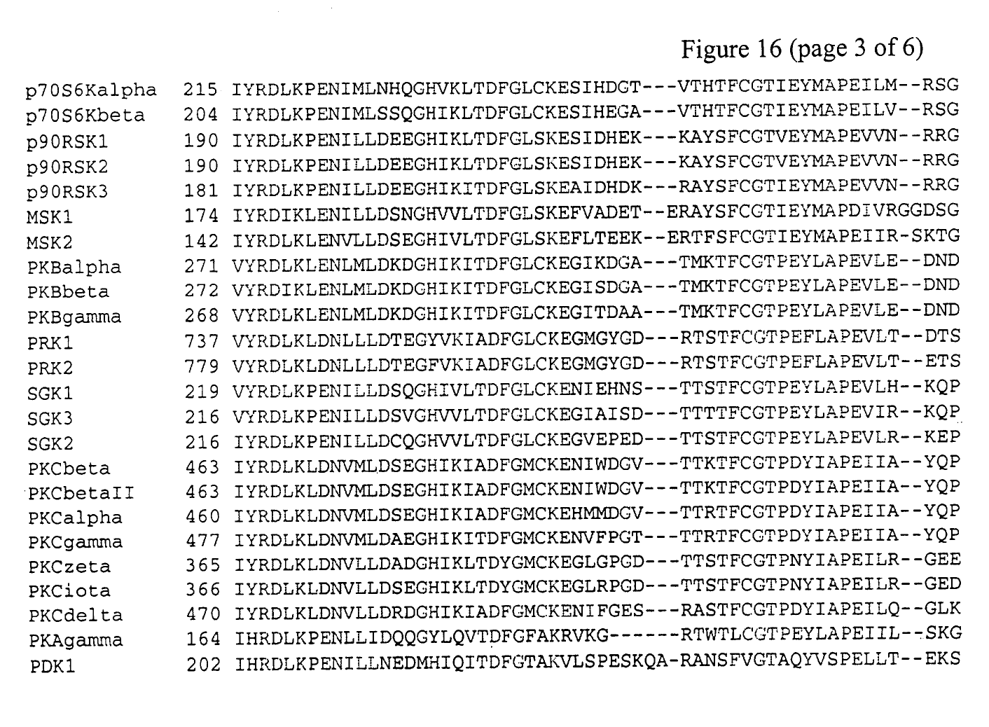

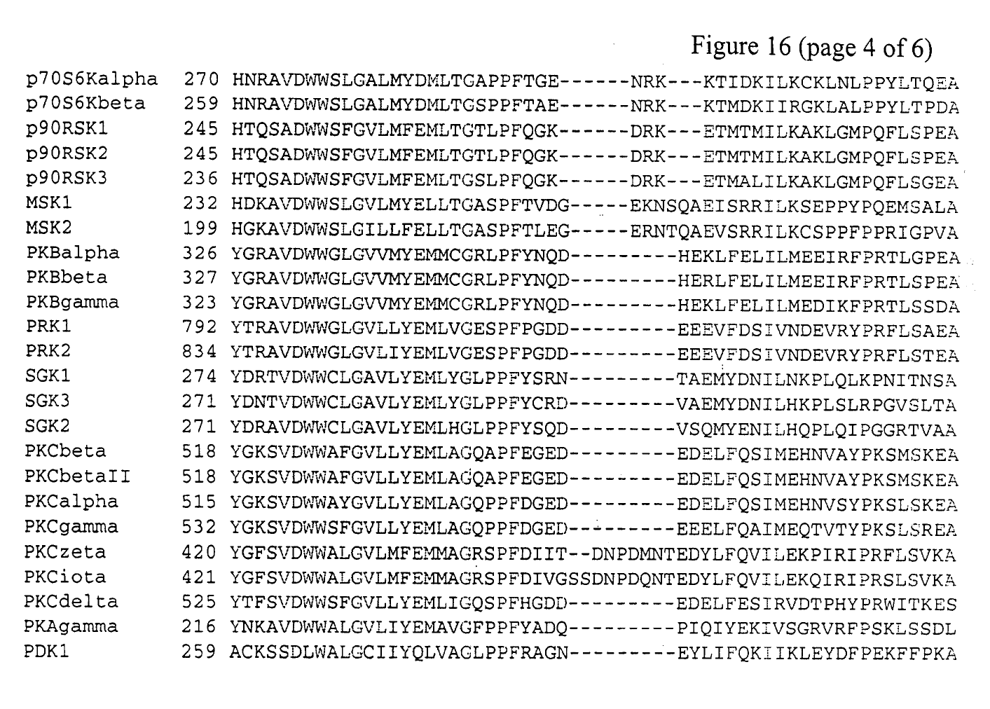

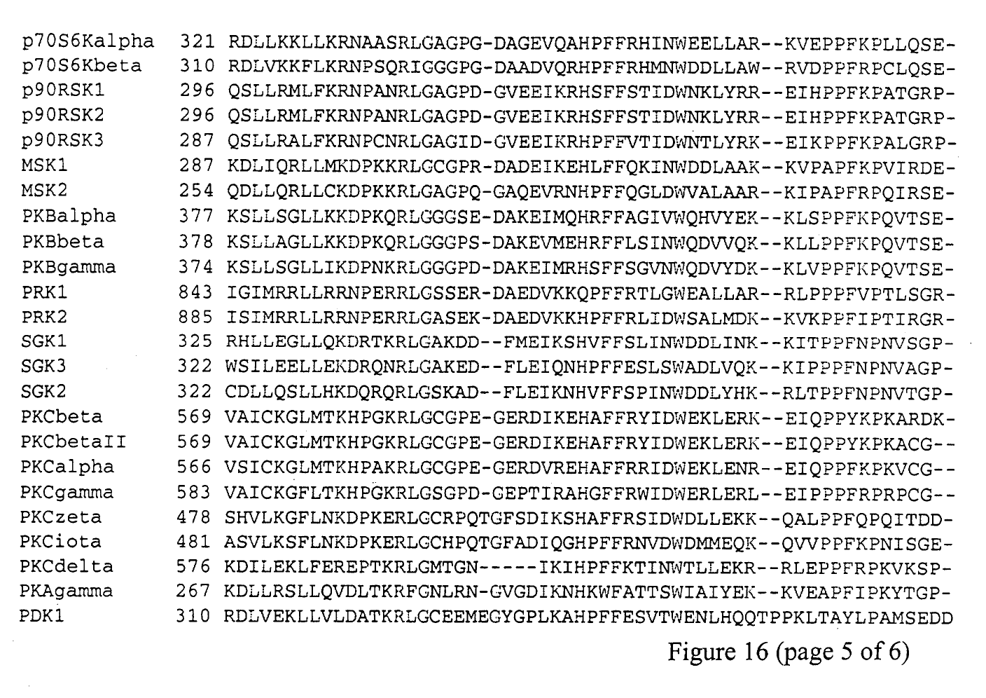

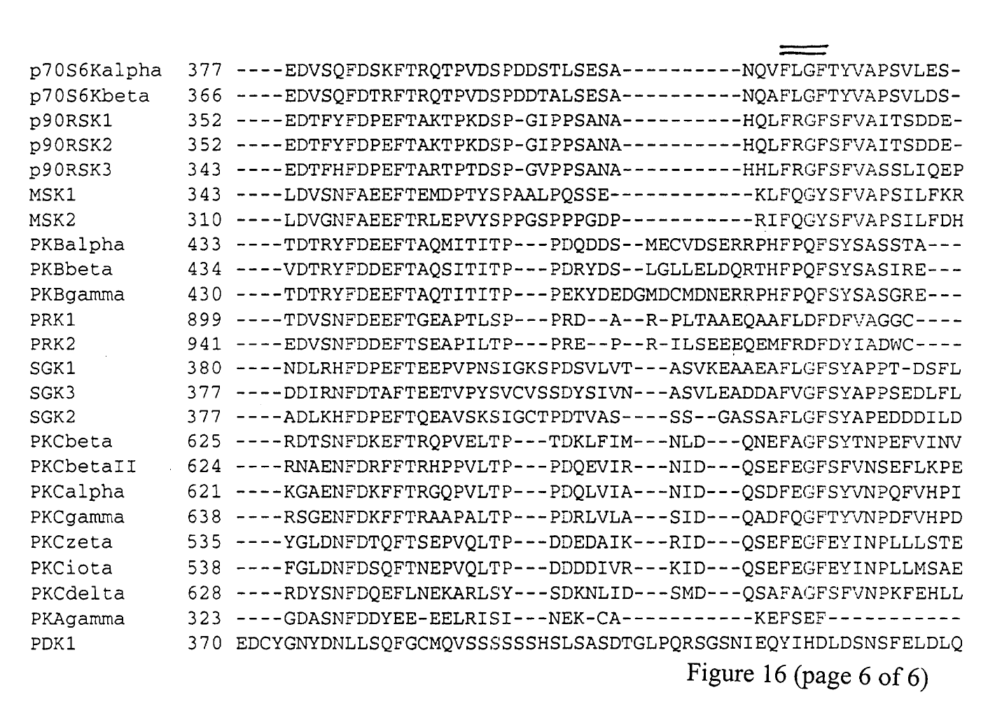

The hydrophobic pocket-containing protein kinase may be the protein kinase termed 3-phosphoinositide-dependent protein kinase 1 (PDK1). Alternatively, it may be Serum and Glucocorticoid stimulated protein kinase (SGK), Protein Kinase B (PKB), Protein Kinase A (PKA), p70 S6 kinase, p90 RSK, PKC isoforms (for example PKCα, PKCδ, PKCζ, PRK1, PRK2, MSK1 or MSK2. Hydrophobic pocket-containing protein kinases and their EMBL database accession numbers are listed in Table I and shown in FIGS. 15 and 16. All AGC family protein kinases may be hydrophobic pocket-containing protein kinases, as defined above. In addition to the protein kinases shown in FIGS. 15 and 16, rhodopsin and G-protein coupled receptor protein kinases, for example, also have a hydrophobic pocket as defined above and the residue equivalent to Lys76 of mouse PKA is a lysine residue.

The term PDK1 as used herein includes a polypeptide (a PDK1 polypeptide) comprising the amino acid sequence identified as PDK1 in Alessi D. R et al (1997) Curr. Biol. 7: 261-269, Alessi D. R et al (1997) Curr. Biol. 7: 776-789, Stokoe D et al (1997) Science 277: 567-570 or Stephens L et al (1998) Science 279: 710-714, or a variant, fragment, fusion or derivative thereof, or a fusion of a said variant or fragment or derivative, for example as described in WO98/41638, incorporated herein by reference. It is preferred that the said PDK1 polypeptide is a protein kinase. It is preferred that the said PDK1 polypeptide is a protein kinase that is capable of phosphorylating a threonine residue that lies in a Thr-Phe-Cys-Gly-Thr-Xaa-Glu-Leu consensus motif (where the underlined Thr corresponds to the threonine that is phosphorylated by PDK1 and Xaa is a variable residue), and preferably that is capable of phosphorylating PKB, for example PKBα, at residue Thr308. The rate at which the said PDK1 polypeptide is capable of phosphorylating a threonine residue as described above may be increased in the presence of PtdIns(3,4,5)P3 or PtdIns(3,4)P₂ but it will be appreciated that this is not essential. The said polypeptide may be capable of phosphorylating the equivalent residues to Thr308 of PKBα on PKC isoforms (LeGood et al (1998) Science 281: 2042-2045; et al (1998) Curr. Biol. 8: 1069-1077; Dutil et al (1998) Curr. Biol. 8:1366-1375), p70 S6 kinase (Alessi et al (1998) Curr. Biol. 8: 69-81; Pullen et al (1998) Science 279, 707-710), SGK (sequence given in Webster et al (1993) Mol. Cell. Biol. 13, 1031-2040; equivalent residues identified in U.S. application Ser. No. 112,217 filed on Dec. 14, 1998; GB 9919676.8, filed on Aug. 19, 1999, and Kobayashi & Cohen (1999)) and PKA (Cheng et al (1998) Proc. Natl. Acad. Sci. USA 95: 9849-9854). It may further be preferred that the substrate specificity and/or other characteristics of the said PDK1 polypeptide in vitro may be substantially as reported in Alessi D. R et al (1997) Curr. Biol. 7: 261-269, Alessi D. R et al (1997) Curr. Biol. 7: 776-789, Stokoe D et al (1997) Science 277: 567-570 or Stephens L et al (1998) Science 279: 710-714.

The terms SGK, PKB, PKA, p70 S6 kinase, p90 RSK, PKCα, PKCδ, PKCζ or PRK2, for example, as used herein include a polypeptide (a SGK, PKB, PKA, p70S6 kinase, p90 RSK, PKCα, PKCδ, PKCζ or PRK2 polypeptide) comprising the amino acid sequence identified as a SGK, PKB, PKA, p70 S6 kinase, p90 RSK, PKCα, PKCδ, PKCζ or PRK2, respectively, in the relevant EMBL database records indicated in Table I.

TABLE I

| | | |
|---|---|---|
| | AGC | |
| Activation or T- | Hydrophobic | Accession |
| Loop | Motif | number |

| | | |
|---|---|---|
| consensus: | TFCGTxxYxAPD | FxxFSY |

| | | |
|---|---|---|
| L | E | YTF |

| | | | |
|---|---|---|---|
| | TFCGTPEYLAPE | FPQFSY | (Y15056) |
| PKBα | | | |
| PKBβ | TFCGTPEYLAPE | FPQFSY | (P31751) |
| PKBγ | TFCGTPEYLAPE | FPQFSY | (AF135794) |
| SGK1 | TFCGTPEYLAPE | FLGFSY | (AAD41091) |
| SGK2 | TFCGTPEYLAPE | FLGFSY | (AF169034) |
| SGK3 | TFCGTPEYLAPE | FLGFSY | (AF169035) |
| PKCα | TFCGTPDYIAPE | FEGFSY | (4506067) |
| PKCβI | TFCGTPDYIAPE | FAGFSY | (4506069) |
| PKCβII | TFCGTPDYIAPE | FEGFSF | (P05127) |
| PKCγ | TFCGTPDYIAPE | FGGFTY | (P05129) |
| PKCδ | TFCGTPDYIAPE | FAGFSF | (5453970) |
| PCKζ | TFCGTPNYIAPE | FEGFEY | (4506079) |
| PKCι | TFCGTPNYIAPE | FEGFEY | (4506071) |
| PRK1 | TFCGTPEFLAPE | FLDFDF | (AAC50209) |
| PRK2 | TFCGTPEFLAPE | FRDFDY | (AAC50208) |
| p70-S6Kα | TFCGTIEYMAPE | FLGFTY | (AAA36410) |
| p70-S6Kβ | TFCGTIEYMAPE | FLGFTY | (4506739) |
| p90-RSK1 | SFCGTVEYMAPE | FRGFSF | (I38556) |
| p90-RSK2 | SFCGTVEYMAPE | FRDFSF | (P51812) |
| p90-RSK3 | SFCGTIEYMAPE | FRGFSF | (CAA59427) |

TABLE II
Alignment of the amino acid sequences surrounding the T-
loop and the hydrophobic motif of AGC kinases.

| MSK1 | SFCGTIEYMAPD | FQGYSF | (AAC31171) |
|---|---|---|---|
| MSK2 | SFCGTIEYMAPE | FQGYSF | (AAC67395) |
| PKA | TLCGTPEYLAPE | FSEF (1) | (P22612) |
| PDK1 | SFVGTAQYVSPE | (2) | (AF017995) |

All the sequences and accession numbers are from human proteins.
The underlined residues correspond to those that become phosphorylated.
Footnotes:
(1) the PKA protein terminates at this position
(2) PDK1 does not possess a hydrophobic motif.

It is particularly preferred, although not essential, that the variant or fragment or derivative or fusion of the PDK1, or the fusion of the variant or fragment or derivative has at least 30% of the enzyme activity of full-length human PDK1 with respect to the phosphorylation of full-length human PKBα or SGK1 on residue Thr308 in either the presence or absence of PtdIns(3,4,5)P₃ or PtdIns(3,4)P₂. It is more preferred if the variant or fragment or derivative or fusion of the said protein kinase, or the fusion of the variant or fragment or derivative has at least 50%, preferably at least 70% and more preferably at least 90% of the enzyme activity of PDK1 with respect to the phosphorylation of PKBα or SGK1. However, it will be appreciated that variants or fusions or derivatives or fragments which are devoid of enzymatic activity may nevertheless be useful, for example by interacting with another polypeptide. Thus, variants or fusions or derivatives or fragments which are devoid of enzymatic activity may be useful in a binding assay, which may be used, for example, in a method of the invention in which modulation of an interaction of PDK1 (as defined above) with a interacting polypeptide, for example an interacting polypeptide comprising the amino acid sequence motif Phe/Tyr-Xaa-Xaa-Phe/Tyr, for example Phe/Tyr-Xaa-Xaa-Phe/Tyr-Zaa-Phe/Tyr, for example Phe/Tyr-Xaa-Xaa-Phe/Tyr-Asp/Glu-Phe/Tyr or Phe/Tyr-Xaa-Xaa-Phe/Tyr-PhosphoSer/PhosphoThr-Phe/Tyr is measured.

It is preferred that the variant or fragment or derivative or fusion of the said hydrophobic pocket-containing protein kinase, or the fusion of the variant or fragment or derivative comprises a hydrophobic pocket in the position equivalent to the hydrophobic pocket of (mouse) PKA that is defined by residues including Lys76, Leu116, Val80 and/or Lys111 of fill-length mouse PKA, as discussed further below.

Equivalent preferences apply to a variant or fragment or derivative or fusion of the SGK, PKB, PKA, p70 S6 kinase, p90 RSK, PKCα, PKCδ, PKCζ or PRK2 (for example), or the fusion of the variant or fragment or derivative, with the substitution in relation to SGK, PKB and p70S6 kinase of the peptide substrate Crosstide (GRPRTSSFAEG), or for PKB and SGK of the peptide substrate RPRAATF; the subsitution in relation to PKA of the peptide substrate Kemptide (LRRASLG); the substitution in relation to PKC isoforms and PRK1/2 of histone H1; and the substitution in relation to MSK1/2 or p90-RSK1/2/3 of CREBtide (EILSRRPSYRK).

By “variants” of a polypeptide we include insertions, deletions and substitutions, either conservative or non-conservative. In particular we include variants of the polypeptide where such changes do not substantially alter the activity of the said polypeptide, for example the protein kinase activity of PDK1, as described above.

By “conservative substitutions” is intended combinations such as Gly, Ala; Val, Ile, Leu; Asp, Glu; Asn, Gln; Ser, Thr; Lys, Arg; and Phe, Tyr.

It is particularly preferred if the PDK1 (or SGK, PKB, PKA or p70 S6 kinase or other hydrophobic pocket-containing kinase as defined above) variant has an amino acid sequence which has at least 65% identity with the amino acid sequence of PDK1 referred to above (or the sequence for SGK (including SGK1, 2 and 3), PKB, PKA or p70 S6 kinase, for example, as appropriate, referred to above), more preferably at least 70%, 71%, 72%, 73% or 74%, still more preferably at least 75%, yet still more preferably at least 80%, in further preference at least 85%, in still further preference at least 90% and most preferably at least 95% or 97% identity with the amino acid sequence defined above.

It is still further preferred if the PDK1 (or SGK, PKB, PKA or p70 S6 kinase or other hydrophobic pocket-containing kinase, as defined above) variant has an amino acid sequence which has at least 65% identity with the amino acid sequence of the catalytic domain, particularly the residues forming the hydrophobic pocket, of PDK1 (or, for example, SGK, PKB, PKA or p70 S6 kinase) in the appropriate sequence referred to above, more preferably at least 70%, 71%, 72%, 73% or 74%, still more preferably at least 75%, yet still more preferably at least 80%, in further preference at least 83 or 85%, in still further preference at least 90% and most preferably at least 95% or 97% identity with the amino acid sequence defined above. It will be appreciated that the catalytic domain of a protein kinase-related polypeptide may be readily identified by a person skilled in the art, for example using sequence comparisons as described below.

The percent sequence identity between two polypeptides may be determined using suitable computer programs, for example the GAP program of the University of Wisconsin Genetic Computing Group and it will be appreciated that percent identity is calculated in relation to polypeptides whose sequence has been aligned optimally.

The alignment may alternatively be carried out using the Clustal W program (Thompson et al (1994) Nucl Acid Res 22, 4673-4680). The parameters used may be as follows:

Fast pairwise alignment parameters: K-tuple(word) size; 1, window size; 5, gap penalty; 3, number of top diagonals; 5. Scoring method: x percent.

Multiple alignment parameters: gap open penalty; 10, gap extension penalty; 0.05.

Scoring matrix: BLOSUM.

It is preferred that the PDK1 (or, for example, SGK, PKB, PKA or p70 S6 kinase) is a polypeptide which consists of the amino acid sequence of the protein kinase PDK1 (or, for example, SGK, PKB, PKA or p70 S6 kinase as the case may be) sequence referred to above or naturally occurring allelic variants thereof. It is preferred that the naturally occuring allelic variants are mammalian, preferably human, but may alternatively be homologues from parasitic or pathogenic or potentially pathogenic organisms. Examples of such organisms and homologues, and of uses of modulators of such homologues are given in U.S. patent application No. 60/112,114, filed on Dec. 14, 1998, and applications claiming priority therefrom, or in Casamayor et al (1999) Curr Biol 9, 186-197.

The PDK1 may also be a polypeptide with the amino acid sequence of residues 51 to 404 of full-length human PDK1; this may comprise the protein kinase domain of PDK1, as described in Example 2. The PDK1 (or SGK, PKB, PKA or p70 S6 kinase) may also be Myc epitope-tagged or His-tagged, as described in Example 1. The p70 S6 kinase, for example, may have a His tag at its N-terminus and/or may lack the carboxy terminal 104 residues (p70 S6K-T2; see Example 1). The PDK1 or SGK may be a Saccharomyces cerevisiae homologue, for example Pkh1 or Pkh2 (PDK1 homologues) or Ypk1 or Yrk2 (SGK homologues), as described in Casamayor et al (1999) Curr Biol 9, 186-197.

It is preferred that the PDK1 (or, for example, SGK, PKB, PKA or p70 S6 kinase) is a polypeptide that is capable of interacting with a polypeptide comprising the amino acid sequence motif Phe/Tyr-Xaa-Xaa-Phe/Tyr, preferably Phe-Xaa-Xaa-Phe/Tyr, more preferably Phe-Xaa-Xaa-Phe, still more preferably Phe/Tyr-Xaa-Xaa-Phe/Tyr-Zaa-Phe/Tyr or Phe/Tyr-Xaa-Xaa-Phe/Tyr-COOH, for example the polypeptide PIF or PIFtide, as defined below. Further preferences for the said polypeptide are as given above in relation to the interacting polypeptide.

The capability of the said PDK1 (or, for example, SGK, PKB, PKA or p70 S6 kinase) polypeptide with regard to interacting with or binding to a polypeptide, for example a polypeptide comprising the amino acid sequence motif Phe/Tyr-Xaa-Xaa-Phe/Tyr, for example Phe/Tyr-Xaa-Xaa-Phe/Tyr-Zaa-Phe/Tyr or Phe/Tyr-Xaa-Xaa-Phe/Tyr-COOH, may be measured by any method of detecting/measuring a protein/protein interaction, as discussed further below. Suitable methods include methods analagous to those discussed above and described in Example 1 or Example 2, for example yeast two-hybrid interactions, co-purification, ELISA, co-immunoprecipitation and surface plasmon resonance methods. Thus, the said PDK1 (or SGK, PKB, PKA or p70 S6 kinase) may be considered capable of binding to or interacting with a polypeptide, for example a polypeptide comprising the amino acid sequence motif Phe/Tyr-Xaa-Xaa-Phe/Tyr, for example Phe/Tyr-Xaa-Xaa-Phe/Tyr-Zaa-Phe/Tyr, Phe/Tyr-Xaa-Xaa-Phe/Tyr-Asp/Glu-Phe/Tyr or Phe/Tyr-Xaa-Xaa-Phe/Tyr-PhosphoSer/PhosphoThr-Phe/Tyr if an interaction may be detected between the said PDK1 polypeptide and the said interacting polypeptide by ELISA, co-immunoprecipitation or surface plasmon resonance methods or by a yeast two-hybrid interaction or copurification method, for example as described in Example 1 or Example 2.

It is preferred that the interaction can be detected using a surface plasmon resonance method, as described in Example 1 or 2 and in Balendran et al (1999), supra and GB9906245.7, supra. The interacting polypeptide may be immobilised on the test surface, for example it can be coupled through amino groups to a SensorChip CM5™, according to the manufacturer's instructions, or a biotinylated polypeptide can be bound to an avidin coated SensorChip SA. The protein kinase (at concentrations between, for example 0 and between 10 μM and 1.0 μM, for example 2 μM) is then injected over the surface and steady state binding determined in each case. From these measurements a K_{d} can be determined. It is preferred that the interaction has a K_{d} of less than 8 μM, more preferably less than 5 μM, 2 μM, 1 μM, 500 nM, 300 nM, 200 nM or 100 nM, for example about 150 nM. The K_{d} of the interaction determined between GST-PDK1 and PIF may be about 150 nM. Alternatively, a K_{d} can be determined for a polypeptide in competition with the immobilised polypeptide. The protein kinase (for example at a concentration of 0.5 μM) is mixed with free polypeptide (for example, at concentrations between 0 and 3 μM) and the mixture injected over the immobilised polypeptides. The steady state binding is determined in each case, from which the K_{d} of the interaction can be determined using the Cheng-Prescott relationship. Alternatively, the interaction may be expressed in terms of an observed response or relative observed responses, measured in terms of mass of protein bound to the surface, as described in Example 2. For example, the polypeptide may be immobilised by amino coupling to a SensorChip CM5 and each protein kinase (for example different mutated protein kinases, as discussed below) for example at a concentration of 1.0 μM, injected over the immobilised polypeptide. Alternatively, the polypeptide may be immobilised on a SA SensorChip and each protein kinase, for example at a concentration of 40 nM injected over the immobilised polypeptide. The steady state response for each protein kinase is determined, for example expressed in Response Units (RU). 1000 RU corresponds to 1 ng/mm² of protein bound to the surface. A response of less than 10 RU may indicate that no interaction has taken place. A response of at least 10 RU may indicate that the immobilised and injected molecules interact with each other.

It will be appreciated that the above methods may be used to determine whether a particular polypeptide is an interacting polypeptide in respect of a protein kinase having a hydrophobic pocket in the position equivalent to the hydrophobic pocket of mouse Protein Kinase A (PKA) that is defined by residues including Lys76, Leu116, Val80 and/or Lys111 of full-length mouse PKA, for example a naturally occuring said hydrophobic pocket-containing protein kinase.

By “protein kinase having a hydrophobic pocket in the position equivalent to the hydrophobic pocket of mouse Protein Kinase A (PKA) that is defined by residues including Lys76, Leu116, Val80 and/or Lys111 of full-length mouse PKA” is meant a polypeptide having an amino acid sequence identifiable as that of a protein kinase catalytic domain, and further having a predicted or determined three-dimensional structure that includes a hydrophobic pocket corresponding to the region indicated in Knighton et al (1991) Science 253, 407-414 for PKA as interacting with C-terminal amino acids of full-length PKA, for example Phe348 and/or Phe351, as discussed in Example 2. The hydrophobic pocket in PKA is in the small lobe of the catalytic domain and does not overlap with the ATP or peptide substrate binding sites on PKA.

Residues Lys76, Val80, Lys111 and/or Leu116 in a hydrophobic pocket of PKA may interact with residues Phe347 and Phe350 at the C-terminus of full length mouse PKA (Uhler et al (1996) PNAS USA 83, 1300-1304). It is preferred that the protein kinase has identical or conserved residues that are equivalent to Lys76, Val80, Lys111 and/or Leu116 of mouse PKA, more preferably at least Lys76 and Leu116 of mouse PKA, most preferably an identical residue equivalent to Lys76. Thus, for example, the protein kinase may have a Lys residue at the position equivalent to Lys76 of PKA and/or a Leu residue at the position equivalent to Leu116 of PKA. Lys115 and Leu155 of PDK1, for example, are equivalent to Lys76 and Leu116, respectively, of PKA. It is preferred that the protein kinase does not have an Ala at the position equivalent to Lys76 and/or a Ser, Asp or Glu at the position equivalent to Leu116 of PKA. The protein kinase may have a Val residue at the position equivalent to Leu116 of PKA, as in PRK1 and 2 (see FIGS. 15 and 16), or an Ile residue. The protein kinase may have a non-conserved residue at the position equivalent to Lys111, for example a glutamine residue and/or at the position equivalent to Val80.

FIGS. 15 and 16 shows an alignment of examples of protein kinases having a hydrophobic pocket at the position equivalent to the said hydrophobic pocket of PKA. The residues equivalent to Lys76, Val80, Lys111 and Leu116 of full length mouse PKA are indicated. A Lys residue is present at the position equivalent to Lys76 of mouse PKA in all of the aligned sequences.

An amino acid sequence may be identifiable as that of a protein kinase catalytic domain by reference to sequence identity or similarities of three dimensional structure with known protein kinase domains, as known to those skilled in the art.

Protein kinases show a conserved catalytic core, as reviewed in Johnson et al (1996) Cell, 85, 149-158 and Taylor & Radzio-Andzelm (1994) Structure 2, 345-355. This core folds into a small N-terminal lobe largely comprising anti-parallel β-sheet, and a large C-terminal lobe which is mostly α-helical. A deep cleft at the interface between these lobes is the site of ATP binding, with the phosphate groups near the opening of the cleft.

Protein kinases also show conserved sequences within this catalytic core, and the residue equivalent to a given residue of, for example, PKA, may be identified by alignment of the sequence of the kinase with that of known kinases in such a way as to maximise the match between the sequences. The alignment may be carried out by visual inspection and/or by the use of suitable computer programs, for example the GAP program of the University of Wisconsin Genetic Computing Group, which will also allow the percent identity of the polypeptides to be calculated. The Align program (Pearson (1994) in: Methods in Molecular Biology, Computer Analysis of Sequence Data, Part II (Griffin, AM and Griffin, HG eds) pp 365-389, Humana Press, Clifton).

The comparison of amino acid sequences or three dimension structure (for example from crystallography or computer modelling based on a known structure) may be carried out using methods well known to the skilled man, as detailed below and as described in Example 2.

MAP kinase, MEK1, Cdk2 and Erk2 (for example) are not protein kinases having a hydrophobic pocket in the position equivalent to the hydrophobic pocket of Protein Kinase A (PKA) that is defined by residues including Lys76, Leu116, Val80 and/or Lys111 of full-length mouse PKA. MEK1, Cdk2 and ERK2 have histidine, glutamine and proline, respectively at the residue equivalent to Lys76 of fill-length mouse PKA ie not lysine or a conservative substitution, and do not interact with a Phe-Xaa-Xaa-Phe amino acid sequence. MEK1, Cdk2 and ERK2 may have a larger hydrophobic pocket which interacts with an amino acid sequence motif (which may be Phe-Xaa-Phe-Pro) which is not Phe-Xaa-Xaa-Phe. Thus, these protein kinases do not have a hydrophobic pocket in the position equivalent to the said hydrophobic pocket of protein kinase A.

The interacting polypeptide may interact with the said hydrophobic pocket of the protein kinase. Thus, it is preferred that the interacting polypeptide interacts with the protein kinase but interacts less strongly with the protein kinase in which one or more residues forming the said hydrophobic pocket is mutated, preferably to a non-conserved amino acid. Most preferably, the mutated residues are the residues equivalent to residues Lys76, Val80, Lys111 and/or Leu116 in the hydrophobic pocket of PKA that is defined by residues including Lys76, Leu116, Val80 and/or Lys111 of full-length mouse PKA. It is particularly preferred that the residue at the position equivalent to residue Lys76 of PKA is mutated to an Ala and/or that the residue at the position equivalent to Leu116 of PKA is mutated to a Ser, Asp or Glu.

It will be appreciated that the interacting polypeptide may interact with additional regions of the protein kinase. For example, it may interact (for example via the acidic residue or group in the preferred amino acid sequence indicated above) with a residue equivalent to Gln35 of PKA (in the N-terminal non-catalytic region of PKA), which appears to form a hydrogen bond with the C-terminal carboxylate group of Phe350, when the C-terminus of PKA interacts with the hydrophobic pocket of PKA.

The interaction may be measured by any of the methods discussed above. In particular, it may be measured using surface plasmon resonance, as discussed above and in Example 1 and 2. It is particularly preferred that the relative strength of interaction with the protein kinase and the mutated protein kinase is determined by measuring the relative steady state responses, as described above. It is preferred that the response (expressed in RUs) for the unmutated protein kinase is at least 2, 5, 10, 30, 50, 80, 100, 200 or 500 times the response for the mutated protein kinase.

The interacting polypeptide, for example comprising the amino acid sequence Phe/Tyr-Xaa-Xaa-Phe/Tyr may be part of the same polypeptide chain as the protein kinase. For example, PKA comprises the amino acid sequence Phe/Tyr-Xaa-Xaa-Phe/Tyr, and SGK, PKB and p70 S6 kinase comprise the amino acid sequence Phe/Tyr-Xaa-Xaa-Phe/Tyr-Zaa-Phe/Tyr wherein Zaa is phosphoserine or phosphothreonine. Thus, the interaction may be an intramolecular interaction, for example in which the hydrophobic pocket (of the protein kinase domain of the polypeptide) and the interacting portion of the polypeptide, for example a portion of the polypeptide comprising a Phe/Tyr-Xaa-Xaa-Phe/Tyr sequence, within a single polypeptide chain, interact. Alternatively, two or more such polypeptide chains may form a dimer or multimer through intermolecular interactions between, for example, the hydrophobic pocket of one polypeptide chain and the interacting portion of a second polypeptide. Intramolecular interactions can be measured by techniques known to those skilled in the art, including cross-linking studies, structural studies and fluorescence resonance energy transfer (FRET) methods, in which changes in separation between fluorophores, for example attached to different parts of a molecule, can be measured.

A polypeptide comprising the amino acid sequence Phe/Tyr-Xaa-Xaa-Phe/Tyr-Ser/Thr-Phe/Tyr may interact with a said hydrophobic pocket of a protein kinase with different affinity depending upon the phosphorylation state of the Ser/Thr residue. Thus, the polypeptide may interact with the hydrophobic pocket more strongly when phosphorylated on the Ser/Thr residue than when not so phosphorylated. In the absence of phosphorylation, the interaction may be substantially undetectable using one or more of the methods described above or may be about 2, 5 or 10-fold weaker than when phosphorylated. Thus, for example, an intra- or intermolecular interaction between the SGK, PKB or p70 S6 kinase protein kinase domain and the portion comprising the sequence Phe/Tyr-Xaa-Xaa-Phe/Tyr-Ser/Thr-Phe/Tyr may occur substantially only when the said sequence is phosphorylated on the Ser/Thr residue. The interaction may modulate, for example increase, the activity and/or stability of the protein kinase domain (or entire polypeptide).

It is preferred that the interacting polypeptide, for example comprising the amino acid sequence motif Phe/Tyr-Xaa-Xaa-Phe/Tyr, is a polypeptide that is capable of binding PDK1 and inhibiting its activity towards p70 S6 kinase in substantially the same way as a polypeptide with the amino acid sequence EDVKKHPFFRLIDWSALMDKKVKPPFIPTIRGREDVSNFDDEFTSEAPILTPPREPRILSEEEQEMFRDFDYIADWC (termed PIF) or (GST)-EDVKKHPFFRLIDWSALMDKKVKPPFIPTIRGREDVSNFDDEFTSEAPILTPPREPRILSEEEQEMFRDFDYIADWC (termed GST-PIF) or REPRILSEEEQEMFRDFDYIADWC (termed PIFtide) as described in Example 1, wherein GST represents a glutathione S-transferase portion, as known to those skilled in the art, and the sequence corresponding to the amino acid sequence motif is underlined.

Alternatively or in addition, it is preferred that the interacting polypeptide, for example comprising the amino acid sequence motif Phe/Tyr-Xaa-Xaa-Phe/Tyr, is a polypeptide that is capable of binding PDK1 and increasing its activity towards (ie phosphorylation of the underlined residue of) KTFCGTPEYLAPEVRR (termed T308tide) in substantially the same way as a polypeptide with the amino acid sequence EDVKKHPFFRLIDWSALMDKKVKPPFIPTIRGREDVSNFDDEFTSEAPILTPPREPRILSEEEQEMFRDFDYIADWC (termed PIF) or (GST)-EDVKKHPFFRLIDWSALMDKKVKPPFIPTIRGREDVSNFDDEFTSEAPILTPPREPRILSEEEQEMFRDFDYIADWC (termed GST-PIF) or REPRILSEEEQEMFRDFDYIADWC (termed PIFtide) as described in Example 2.

The three-letter amino acid code of the IUPAC-IUB Biochemical Nomenclature Commission is used herein, with the exception of the symbol Zaa, defined above. In particular, Xaa represents any amino acid. It is preferred that Xaa and Zaa represent a naturally occuring amino acid.

It is preferred that at least the amino acids corresponding to the consensus sequences defined above are L-amino acids.

By modulation of the protein kinase activity is included inhibition or an increase in the protein kinase activity.

The protein kinase activity of PDK1 that is modulated may be phosphorylation of the underlined residue in a polypeptide with the amino acid sequence Thr/Ser-Phe-Cys-Gly-Thr-Xaa-Glu-Leu (“PDK1” activity). Alternatively or in addition, the modulated activity may be phosphorylation of the underlined residue in a polypeptide with the amino acid sequence Phe-Xaa-Xaa-Phe-Ser/Thr-Phe/Tyr (“PDK2” activity). The polypeptide may be, for example, a PKB, SGK, p70 S6 kinase, PKC or (in relation only to phosphorylation of the underlined residue in a polypeptide with the amino acid sequence Thr/Ser-Phe-Cys-Gly-Thr-Xaa-Glu-Leu) PKA polypeptide.

The protein kinase activity of PKA that is modulated may be phosphorylation of the underlined residue in a polypeptide with the amino acid sequence Arg-Arg-X-Ser-Y, wherein X is any small residue and Y is a large hydrophobic group. Substrates of PKA include the transcription factor CREB, which is phosphorylated on Ser133, and the polypeptide BAD, which is phosphorylated on Ser112 and Ser155.

The protein kinase activity of PKB, SGK or p70 S6 kinase that is modulated may be phosphorylation of the underlined residue in a polypeptide with the amino acid sequence Arg-Xaa-Arg-Xaa-Xaa-Ser/Thr. The polypeptide may be Glycogen Synthase Kinase 3 (GSK3), 40 S ribosomal subunit S6, BAD, 6-phosphofructo-2-kinase, phosphodiesterase3b, human caspase 9, endothelial nitric oxide synthase or BRACA1.

A compound identified by a method of the invention may modulate the ability of the protein kinase to phosphorylate different substrates, for example different naturally occuring polypeptides, to different extents. The compound may inhibit the protein kinase activity in relation to one substrate but may increase the protein kinase activity in relation to a second substrate, for example as discussed in Example 2. For example, the protein kinase activity may be modulated to a different extent for PKB when compared with SGK, p70 S6 kinase and/or PKC.

It will be appreciated that the modulatory, for example inhibitory action of a compound found to bind (or inhibit binding of a polypeptide or compound) to the protein kinase may be confirmed by performing an assay of enzymic activity (for example PDK1 and/or PDK2 protein kinase activity) in the presence of the compound.

The said interacting polypeptide may be derivable from PRK1, PRK2, a PKC isoform, for example PKCζ, PKCα or PKCδ, PKA, SGK, p70 S6 kinase or PKB, preferably from the C-terminal portion of PKA, PRK1, PRK2, PKCα, PKCδ or PKCζ. The said interacting polypeptide may be derivable from PRK2 by proteolytic cleavage, for example by Caspase 3, as described in Balendran et al (1999), supra.

Thus, the interacting polypeptide may comprise or consist essentially of the amino acid sequence from residue 701 to the C-terminus of PRK2. This may correspond to the C-terminal 77 amino acids of PRK2, termed the PDK1-Interacting Fragment (PIF; see Balendran et al (1999), supra).

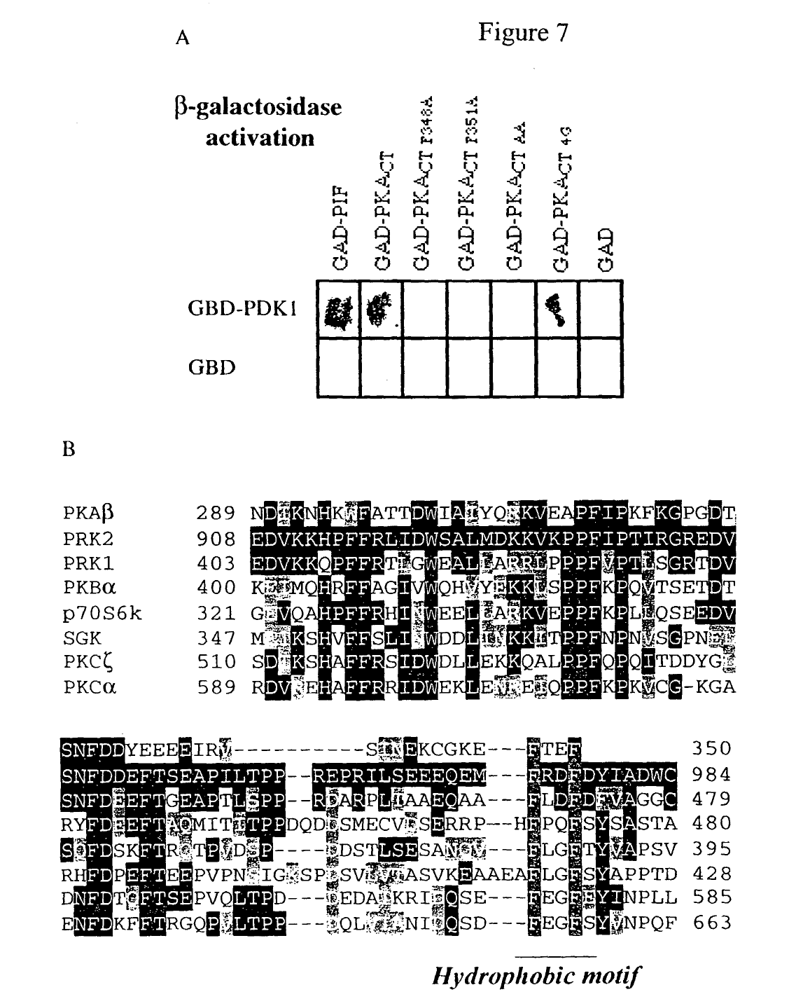

The PIF region of PRK2 may lie immediately C-terminal to the kinase catalytic domain of PRK2. The polypeptide may comprise or consist essentially of the amino acid sequence of residues 960 to 984 of PRK2 (termed Region B) or the equivalent region of PRK1, PRK1, PKBα, p70S6 kinase, PKB, SGK, a PKC isoform, for example PKCζ or PKCα, or PKAβ as shown in FIG. 7E. The polypeptide may comprise or consist of the C-terminal 223 or C-terminal 62 amino acids of PKA, as described in Example 2 and shown in FIG. 7. PKC isoforms are described, for example, in Mellor & Parker (1998) Biochem J 332, 281-292. A polypeptide that comprises an amino acid sequence that corresponds to the consensus sequence Phe/Tyr-Xaa-Xaa-Phe/Tyr-Ser/Thr-Phe/Tyr may interact with PDK1 (1) when the serine or threonine residue is phosphorylated, so that the polypeptide then comprises an amino acid sequence that corresponds to the consensus sequence Phe/Tyr-Xaa-Xaa-Phe/Tyr-PhosphoSer/PhosphoThr-Phe/Tyr, or (2) if the serine or threonine residue is replaced by an aspartate or glutamate residue. PKCδ comprises an amino acid sequence corresponding to the consensus sequence Phe/Tyr-Xaa-Xaa-Phe/Tyr-Ser/Thr-Phe/Tyr (see FIG. 15) and may interact with PDK1 when unphosphorylated.

The said interacting polypeptide may comprise or consist essentially of the sequence REPRILSEEEQEMFRDFDYIADWC or REPRILSEEEQEMARDFDYIADWC or REPRILSEEEQEMFGDFDYIADWC. The said interacting polypeptide may further comprise the sequence EDVKKHPFFRLIDWSALMDKKVKPPFIPTIRGREDVSNFDDEFTSEAPILTPP (see Balendran et al (1999), supra and GB 9906245.7).

The said interacting polypeptide may comprise or consist essentially of a variant of a sequence indicated above. Preferably, in such a variant, the residues that correspond to the amino acid sequence Phe/Tyr-Xaa-Xaa-Phe/Tyr in the sequence indicated above are unchanged, or, if changed, still have the sequence Phe/Tyr-Xaa-Xaa-Phe/Tyr. It is preferred that the residues within about 2, 5 or 10 amino acids C- or N-terminal of the said sequence are also unchanged. It is preferred that the interacting polypeptide has fewer than about 400, 380, 350, 300, 250, 200, 150, 100, 80, 50, 40 or 30 amino acids.

The said interacting polypeptide may comprise a GST portion, as described in Examples 1 and 2. This may be useful in purifying and/or detecting the said interacting polypeptide. The said interacting polypeptide may be biotinylated or otherwise tagged, for example with a 6His, HA, myc or other epitope tag, as known to those skilled in the art.

A further aspect of the invention provides a said interacting polypeptide immobilised on a surface of an article suitable for use as a test surface in a surface plasmon resonance method. The surface may be a SensorChip™ surface, for example a SensorChip CM5™ or SA SensorChip™ surface. It is preferred that the interacting polypeptide is not PIF or PIFtide. It is further preferred that the interacting polypeptide comprises the amino acid sequence Phe/Tyr-Xaa-Xaa-Phe/Tyr and does not comprise the amino acid sequence Phe/Tyr-Xaa-Xaa-Phe/Tyr-Zaa-Phe/Tyr or Phe/Tyr-Xaa-Xaa-Phe/Tyr-Ser/Thr-Phe/Tyr. It is preferred that the interacting polypeptide has fewer than about 400, 380, 350, 300, 250, 200, 150, 100, 80, 50, 40 or 30 amino acids.

The ability of the compound to inhibit or promote the interaction of the said protein kinase with the interacting polypeptide may be measured by detecting/measuring the interaction using any suitable method and comparing the interaction detected/measured in the presence of different concentrations of the compound, for example in the absence and in the presence of the compound, for example at a concentration of about 100 μM, 30 μM, 10 μM, 3 μM, 1 μM, 0.1 μM, 0.01 μM and/or 0.001 μM. Suitable methods include methods analagous to those discussed above and described in Example 1 or Example 2, for example yeast two-hybrid interactions, co-purification, ELISA, co-immunoprecipitation and surface plasmon resonance methods.

A further aspect of the invention provides a method of identifying a compound that modulates the protein kinase activity of a protein kinase having a hydrophobic pocket in the position equivalent to the hydrophobic pocket of Protein Kinase A (PKA), wherein the effect of the said compound on the rate or degree of phosphorylation of a substrate polypeptide of the said hydrophobic pocket-containing protein kinase by the said hydrophobic pocket-containing protein kinase in the presence of an interacting polypeptide is determined, and a compound that modulates the said rate or degree of phosphorylate is selected, wherein the interacting polypeptide interacts with the hydrophobic pocket of the protein kinase and/or comprises the amino acid sequence Phe/Tyr-Xaa-Xaa-Phe/Tyr and is comprised in a separate polypeptide chain to the hydrophobic pocket-containing protein kinase, and wherein the substrate polypeptide has fewer than 400 amino acids, still more preferably fewer than 380, 350, 300, 250, 200, 150, 120, 100, 80, 70, 60, 50, 40, 30, 25 or 20 amino acids.

The effect of the compound may be determined by comparing the rate or degree of phosphorylation of the said substrate polypeptide by the said hydrophobic pocket-containing protein kinase in the presence of different concentrations of the compound, for example in the absence and in the presence of the compound, for example at a concentration of about 100 μM, 30 μM, 10 μM, 3 μM, 1 μM, 0.1 μM, 0.01 μM and/or 0.001 μM.

The substrate polypeptide may comprise a portion that is the interacting polypeptide, for example that comprises the amino acid sequence Phe/Tyr-Xaa-Xaa-Phe/Tyr. Thus, the substrate polypeptide may comprise non-overlapping interacting and substrate portions. The substrate polypeptide may comprise (1) an interacting portion, for example comprising the amino acid sequence Phe/Tyr-Xaa-Xaa-Phe/Tyr and (2) a substrate portion comprising a consensus sequence for phosphorylation by a protein kinase having a hydrophobic pocket in the position equivalent to the said hydrophobic pocket of Protein Kinase A (PKA), for example PDK1, PKB, SGK, p70 S6 kinase or PKA, for example the sequence Thr/Ser-Phe-Cys-Gly-Thr-Xaa-Glu-Leu, Phe-Xaa-Xaa-Phe-Ser/Thr-Phe/Tyr, Arg-Arg-X-Ser-Val or Arg-Xaa-Arg-Xaa-Xaa-Ser/Thr. It is preferred that the amino acid sequences indicated in relation to the said substrate and interacting portions are separated by between about 1 and 100 to 150 amino acids, preferably between about 5 and 50, still more preferably between about 10 and 30 amino acids, for example about 26 amino acids.

A further aspect of the invention provides a substrate polypeptide as defined above wherein the amino acid sequence indicated in relation to the said substrate and interacting portions are separated by between about by between about 1 and 100 to 150 amino acids, preferably between about 5 and 50, still more preferably between about 10 and 30 amino acids, for example about 26 amino acids.

Thus, if the hydrophobic pocket-containing protein kinase is PDK1, the substrate polypeptide may comprise the sequence KTFCGTPEYLAPEV (substrate portion) and, for example, the sequence EPRILSEEEQEMFRDFDYIADWC (interacting polypeptide portion, for example hydrophobic pocket binding portion). The substrate polypeptide may, for example, comprise or consist of the sequence KTFCGTPEYLAPEVRREPRILSEEEQEMFRDFDYIADWC.

Alternatively, the substrate portion and the interacting portion may be present on separate polypeptide chains, ie as separate substrate polypeptide and interacting polypeptide. Thus, if the hydrophobic pocket-containing protein kinase is PDK1, the substrate polypeptide may comprise or consist of the sequence KTFCGTPEYLAPEV, and the interacting polypeptide may comprise or consist of the sequence EPRILSEEEQEMFRDFDYIADWC.

It will be appreciated that the compound may interact with PDK1 or with the said interacting polypeptide or with both.

A further aspect of the invention provides a method of identifying a compound that modulates the protein kinase activity of a protein kinase having a hydrophobic pocket in the position equivalent to the hydrophobic pocket of Protein Kinase A (PKA) that is defined by residues including Lys76, Leu116, Val80 and/or Lys111 of full-length mouse PKA wherein the effect of the said compound on the rate or degree of phosphorylation of a substrate polypeptide of the said hydrophobic pocket-containing protein kinase by the said hydrophobic pocket-containing protein kinase is determined, and a compound that modulates the said rate or degree of phosphorylation is selected, wherein the effect of the compound is determined in the absence (including substantial absence) of an interacting polypeptide, wherein an interacting polypeptide interacts with the hydrophobic pocket of the protein kinase and/or comprises the amino acid sequence Phe/Tyr-Xaa-Xaa-Phe/Tyr, and wherein the substrate polypeptide has fewer than 400 amino acids, still more preferably fewer than 380, 350, 300, 250, 200, 150, 120, 100, 80, 70, 60, 50, 40, 30, 25 or 20 amino acids.

Thus, the substrate polypeptide and the hydrophobic pocket-containing protein kinase do not comprise an interacting polypeptide that interacts with the hydrophobic pocket of the protein kinase and/or comprises the amino acid sequence Phe/Tyr-Xaa-Xaa-Phe/Tyr.

The compound may mimic the effect of the interaction of an interacting polypeptide (that interacts with the hydrophobic pocket of the protein kinase and/or comprises the amino acid sequence Phe/Tyr-Xaa-Xaa-Phe/Tyr) with the protein kinase.

The effect of the compound may be determined by comparing the rate or degree of phosphorylation of the said substrate polypeptide by the said hydrophobic pocket-containing protein kinase in the presence of different concentrations of the compound, for example in the absence and in the presence of the compound, for example at a concentration of about 100 μM, 30 μM, 10 μM, 3 μM, 1 μM, 0.1 μM, 0.01 μM and/or 0.001 μM.

Most preferably, the protein kinase is PDK1 and the substrate polypeptide consists of or comprises the amino acid sequence KTFCGTPEYLAPEV or KTFCGTPEYLAPEVRR. A compound that mimics the effect of an interacting polypeptide on PDK1 may increase the rate or extent of phosphorylation of such a substrate polypeptide by PDK1.

By “mimic the effect of the interaction of the said interacting polypeptide with the protein kinase” is meant that the compound has a quantitative or qualitative effect on the hydrophobic pocket-containing protein kinase, for example on its protein kinase activity or stability, that is the same as an effect of the interacting polypeptide on the protein kinase, for example on its protein kinase activity or stability, as discussed in Examples 1 and 2. For example, the interacting polypeptide PIF increases the rate at which PDK1 phosphorylates the polypeptide KTFCGTPEYLAPEVRR; a mimic of PIF may increase the rate at which PDK1 (in the absence of PIF) phosphorylates the said polypeptide.

The protein kinase and interacting polypeptide may form a complex, which may be detected in a cell-free system, for example by BiaCore measurements, as described in Examples 1 and 2. The ability of the compound to inhibit or promote the formation or stability of the complex may be determined by exposing the protein kinase and/or interacting polypeptide and/or complex of the protein kinase and interacting polypeptide to the compound and determining any change in the affinity, extent or stability of the interaction in the presence of the compound. The estimated equilibrium dissociation constant of the association between GST-PIF and His-tagged PDK1 may be 600 nM. The estimated dissociation constant K_{d} between His-PDK1 and an immobilised and biotinylated 24 residue synthetic peptide corresponding to Region B above (PIF) detected using Surface Plasmon Resonance measurements was 800 nM, or 1.5 μM.

It is preferred that the said protein kinase, interacting polypeptide and/or, where appropriate, substrate polypeptide, is a recombinant or synthetic polypeptide. It is further preferred that the said protein kinase, interacting polypeptide and/or, where appropriate, substrate polypeptide is substantially pure when introduced into the method of the invention.

By “substantially pure” we mean that the protein kinase or interacting polypeptide or substrate polypeptide is substantially free of other proteins. Thus, we include any composition that includes at least 30% of the protein content by weight as the said protein kinase or interacting polypeptide or substrate polypeptide, preferably at least 50%, more preferably at least 70%, still more preferably at least 90% and most preferably at least 95% of the protein content is the said protein kinase or interacting polypeptide or substrate polypeptide.

Thus the substantially pure protein kinase or interacting polypeptide or substrate polypeptide may include a contaminant wherein the contaminant comprises less than 70% of the composition by weight, preferably less than 50% of the composition, more preferably less than 30% of the composition, still more preferably less than 10% of the composition and most preferably less than 5% of the composition by weight.

The substantially pure said protein kinase or interacting polypeptide or substrate polypeptide may be combined with other components ex vivo, said other components not being all of the components found in the cell in which said protein kinase or interacting polypeptide or substrate polypeptide is naturally found.

The said protein kinase, for example PDK1 (or SGK, PKB, p70 S6 kinase or PKA), and said interacting polypeptide may be exposed to each other and to the compound to be tested in a cell in which the said protein kinase and the said interacting polypeptide are both expressed, as described in Examples 1 and 2. The protein kinase may be the endogenous protein kinase or it may be a protein kinase expressed from a recombinant construct. Similarly, the said interacting polypeptide may be endogenous or it may be expressed from a recombinant construct, as described in Example 1. The said protein kinase and the said interacting polypeptide are not exposed to each other in a cell in which the said protein kinase and the said interacting polypeptide are both naturally expressed. The said protein kinase and the said interacting polypeptide are not both endogenous polypeptides to the cell in which the said protein kinase and the said interacting polypeptide are exposed to each other.

A complex may also be detected by coimmunoprecipitation or copurification experiments, or using fluorescence resonance energy transfer (FRET) techniques (for example using fusion proteins comprising fluorescent proteins, for example green, blue or yellow fluorescent proteins (GFPs; YFPs, BFPs, as well known to those skilled in the art)), for example in material from cells in which the protein kinase (as defined above) and the said interacting polypeptide are coexpressed, as described in Examples 1 and 2.

A further aspect of the invention provides a compound (termed an interacting compound) capable of modulating the protein kinase activity of a hydrophobic pocket-containing protein kinase as defined above wherein the compound inhibits the interaction of the said protein kinase with an interacting polypeptide, wherein the interacting polypeptide interacts with the hydrophobic pocket of the said protein kinase and/or comprises the amino acid sequence Phe/Tyr-Xaa-Xaa-Phe/Tyr, wherein the compound does not comprise a polypeptide having the amino acid sequence Phe/Tyr-Xaa-Xaa-Phe/Tyr-Zaa-Phe/Tyr and is not PKA or PKCδ.

A further aspect of the invention provides a compound (termed an interacting compound) capable of modulating the protein kinase activity of a hydrophobic pocket-containing protein kinase as defined above, wherein the compound modulates the rate or degree of phosphorylation of a substrate polypeptide of the said hydrophobic pocket-containing protein kinase by the said hydrophobic pocket-containing protein kinase in the absence (including substantial absence) of an interacting polypeptide, wherein an interacting polypeptide interacts with the hydrophobic pocket of the protein kinase and/or comprises the amino acid sequence Phe/Tyr-Xaa-Xaa-Phe/Tyr, and wherein the substrate polypeptide has fewer than 400 amino acids, still more preferably fewer than 380, 350, 300, 250, 200, 150, 120, 100, 80, 70, 60, 50, 40, 30, 25 or 20 amino acids.

A further aspect of the invention provides a compound that modulates the protein kinase activity of a protein kinase having a hydrophobic pocket in the position equivalent to the hydrophobic pocket of Protein Kinase A (PKA) that is defined by residues including Lys76, Leu116, Val80 and/or Lys111 of full-length mouse PKA, wherein the compound modulates the rate or degree of phosphorylation of a substrate polypeptide of the said hydrophobic pocket-containing protein kinase by the said hydrophobic pocket-containing protein kinase in the presence of an interacting polypeptide, wherein the interacting polypeptide interacts with the hydrophobic pocket of the protein kinase and/or comprises the amino acid sequence Phe/Tyr-Xaa-Xaa-Phe/Tyr and is comprised in a separate polypeptide chain to the hydrophobic pocket-containing protein kinase, and wherein the substrate polypeptide has fewer than 400 amino acids, still more preferably fewer than 380, 350, 300, 250, 200, 150, 120, 100, 80, 70, 60, 50, 40, 30, 25 or 20 amino acids.

The compound may be or comprise a polypeptide having the C-terminal sequence Phe-Xaa-Xaa-Phe-COOH, or Phe/Tyr-Xaa-Xaa-Phe/Tyr-(X)ₙ—COOH, preferably Phe-Xaa-Xaa-Phe-(X)ₙ—COOH, wherein n is between 1 and 150, 100, 60, 50, 30, 20, 15, 10, 5, 4, 3 or 2, preferably between 1 and 4, most preferably 4. Each amino acid X is any amino acid residue, preferably glycine. Thus, it is preferred that the polypeptide has the C-terminal sequence Phe-Xaa-Xaa-Phe-COOH or Phe-Xaa-Xaa-Phe-(Gly)₄-COOH. The polypeptide may consist of or comprise contiguous residues derivable from PKA. For example, it may comprise the C-terminal about 223, 220, 200, 180, 160, 140, 120, 100, 80, 70, 65, 63, 55, 50, 45, 40, 35, 30, 25, 20, 15, 10 or 5 amino acids of PKA, or a variant or fusion thereof that has the C-terminal sequence Phe-Xaa-Xaa-Phe-COOH or Phe/Tyr-Xaa-Xaa-Phe/Tyr-(X)ₙ—COOH.

It will be appreciated that the polypeptide may comprise a covalent modification, for example it may be modified by biotinylation ie comprise a biotin group.

A further aspect of the invention provides a compound identifiable by the method of the invention (termed an interacting compound), provided that the compound is not a polypeptide having the amino acid sequence Phe/Tyr-Xaa-Xaa-Phe/Tyr-Zaa-Phe/Tyr and is not full length PKA.

The compound may be, for example, a compound selected on the basis of, or designed to have, as well known to those skilled in the art, a three-dimensional conformation that may be similar to that of an interacting polypeptide, for example comprising the amino acid sequence Phe/Tyr-Xaa-Xaa-Phe/Tyr, for example Phe/Tyr-Xaa-Xaa-Phe/Tyr-COOH, as discussed above.

A further aspect of the invention provides a mutated protein kinase, wherein the protein kinase before mutation has a hydrophobic pocket in the position equivalent to the hydrophobic pocket of Protein Kinase A (PKA) that is defined by residues including Lys76, Leu116, Val80 and/or Lys111 of full-length mouse PKA, and wherein one or more residues forming the hydrophobic pocket of the protein kinase is mutated. It is preferred that the said protein kinase is not PKA. The said protein kinase may be, for example, SGK, PKB, p70 S6 kinase or PDK1, preferably PDK1. It is preferred that the mutated residue(s) are the residues equivalent to residue Lys76, Val80, Lys111 and/or Leu116 in the hydrophobic pocket of PKA. It is particularly preferred that the residue at the position equivalent to residue Lys76 of PKA is mutated to an Ala and/or that the residue at the position equivalent to Leu116 of PKA is mutated to a Ser, Asp or Glu. The equivalent residues of are indicated for several protein kinases in FIG. 15. The mutated protein kinase may be useful in determining whether a polypeptide or compound interacts with the hydrophobic pocket of the unmutated protein kinase, as discussed above and shown in Examples 1, 2 and 3. For example, the abilities of a compound (including polypeptide) to bind to the mutated and unmutated protein kinase, or to modulate the activity of the protein kinase towards one or more substrates of the protein kinase, may be measured and compared.

A further aspect of the invention provides a preparation comprising a protein kinase having a hydrophobic pocket in the position equivalent to the hydrophobic pocket of Protein Kinase A (PKA) that is defined by residues including Lys76, Leu116, Val80 and/or Lys111 of full-length mouse PKA, and a second, interacting compound (encompassing an interacting polypeptide), wherein the interacting compound interacts with the hydrophobic pocket of the protein kinase and/or comprises the amino acid sequence Phe/Tyr-Xaa-Xaa-Phe/Tyr, wherein the said preparation further comprises a substrate polypeptide as defined above and does not comprise all of the components found in a cell in which said protein kinase or compound is naturally found.

A further aspect of the invention provides a preparation comprising a protein kinase having a hydrophobic pocket in the position equivalent to the hydrophobic pocket of Protein Kinase A (PKA) that is defined by residues including Lys76, Leu116, Val80 and/or Lys111 of full-length mouse PKA, and a second, interacting compound, wherein the interacting compound interacts with the hydrophobic pocket of the protein kinase, wherein the said preparation does not comprise all of the components found in a cell in which said protein kinase or compound is naturally found, and wherein when the protein kinase is PDK1, the interacting compound is not a polypeptide comprising the amino acid sequence Phe/Tyr-Xaa-Xaa-Phe/Tyr-Zaa-Phe/Tyr. The interacting compound may be an interacting polypeptide as defined above. Preferences for the interacting polypeptide and protein kinase are as given above. It is preferred that an interacting polypeptide does not comprise the sequence Phe/Tyr-Xaa-Xaa-Phe/Tyr-Ser/Thr-Phe/Tyr. Thus, the preparation may be substantially free of polypeptides with which the protein kinase or compound is present or associated in a cell other than a said interacting polypeptide. The compound may be a compound of the invention that mimics the effect of an interacting polypeptide on the protein kinase.

Thus, we include any composition that includes at least 30% of the protein content by weight as the said protein kinase or interacting polypeptide or (if appropriate) substrate polypeptide (ie in combination), preferably at least 50%, more preferably at least 70%, still more preferably at least 90% and most preferably at least 95% of the protein content is the said protein kinase or interacting polypeptide or (if appropriate) substrate polypeptide.

Thus, the invention also includes preparations comprising the said protein kinase, the said interacting compound, for example polypeptide, and the said substrate polypeptide (if appropriate), and a contaminant wherein the contaminant comprises less than 70% of the composition by weight, preferably less than 50% of the composition, more preferably less than 30% of the composition, still more preferably less than 10% of the composition and most preferably less than 5% of the composition by weight. The invention also includes a preparation comprising the said protein kinase and the said interacting compound, for example polypeptide, and the said substrate polypeptide (if appropriate) when combined with other components ex vivo, said other components not being all of the components found in the cell in which said protein kinase and/or interacting compound, for example polypeptide, and/or substrate polypeptide is naturally found.

A further aspect of the invention provides a method of phosphorylating a substrate polypeptide for a protein kinase having a hydrophobic pocket in the position equivalent to the hydrophobic pocket of Protein Kinase A (PKA) that is defined by residues including Lys76, Leu116, Val80 and/or Lys111 of full-length mouse PKA, wherein a preparation according to the preceding aspect of the invention is used. The substrate polypeptide comprises the appropriate consensus sequence for phosphorylation by a protein kinase having a hydrophobic pocket in the position equivalent to the hydrophobic pocket of Protein Kinase A (PKA) that is defined by residues including Lys76, Leu116, Val80 and/or Lys111 of full-length mouse PKA, for example PDK1, PKB, SGK, p70 S6 kinase or PKA, for example the sequence Thr/Ser-Phe-Cys-Gly-Thr-Xaa-Glu-Leu, Phe-Xaa-Xaa-Phe-Ser/Thr-Phe/Tyr, Arg-Arg-X-Ser-Val or Arg-Xaa-Arg-Xaa-Xaa-Ser/Thr.

When the protein kinase is PDK1, the substrate polypeptide may be PKB, for example PKBα, SGK, p70S6 kinase, PKA or a PKC isoform. When the protein kinase is p70 S6 kinase, the substrate may be ribosomal 40S subunit S6. When the protein kinase is PKB, SGK or p70S6 kinase, the substrate may be glycogen synthase kinase 3 (GSK3), BAD, 6-phosphofructo-2-kinase, phosphodiesterase 3b, human caspase 9, endothelial nitric oxide synthase or BRCA, for example BRCA2.

It will be appreciated that the method may be carried out in the presence of a phosphoinositide, for example PIP₂ or PtdIns(3,4,5)P₃ (PIP₃). The said PIP₂ or PIP₃ may increase the rate or extent of phosphorylation of the underlined residue in a substrate polypeptide with an amino acid sequence corresponding to the consensus sequence Phe-Xaa-Xaa-Phe-Ser/Thr-Phe/Tyr and/or of the residue corresponding to the underlined residue in the consensus sequence Thr/Ser-Phe-Cys-Gly-Thr-Xaa-Glu-Leu, for example by PDK1. The substrate may be PKB, for example PKB comprising a functional (ie phosphoinositide-binding) PH domain.

A further aspect of the invention provides a method of phosphorylating p70 S6 kinase on the residue equivalent to Thr412 of full length human p70 S6 kinase wherein the said p70 S6 kinase is exposed to PDK1. A further aspect of the invention provides the use of PDK1 in a method of phosphorylating p70 S6 kinase on the residue equivalent to Thr412 of full length human p70 S6 kinase. The p70 S6 kinase has a serine or threonine residue at the position equivalent to Thr412 of full length human p70 S6 kinase. The p70 S6 kinase is preferably a naturally occuring p70 S6 kinase, for example full length human p70 S6 kinase, or a fragment or fusion thereof, or a fusion of a fragment thereof, for example as described in Example 1. The p70 S6 kinase and/or the PDK1 are preferably recombinant p70 S6 kinase or PDK1, still more preferably recombinant p70 S6 kinase or PDK1 expressed in a bacterial, yeast or mammalian cell. The method may be performed in vitro or in a cell.

A further aspect of the invention provides a method of identifying a compound that modulates the activation and/or phosphorylation of p70 S6 kinase on the residue equivalent to Thr412 of full length human p70 S6 kinase by PDK1 wherein the activation and/or phosphorylation of p70 S6 kinase on the residue equivalent to Thr412 of full length human p70 S6 kinase by PDK1 is measured in the presence of more than one concentration (for example in the presence or absence) of the compound. A further aspect of the invention is a compound identified or identifiable by the said method.

A further aspect of the invention provides the use of an interacting polypeptide as defined above, for example comprising the amino acid sequence Phe/Tyr-Xaa-Xaa-Phe/Tyr or an interacting compound of the invention in a method of stabilising a protein kinase having a hydrophobic pocket in the position equivalent to the hydrophobic pocket of Protein Kinase A (PKA) that is defined by residues including Lys76, Leu116, Val80 and/or Lys111 of full-length mouse PKA, wherein the protein kinase is exposed to the compound or polypeptide. Stabilisation may be measured by measuring the TM₅₀ value. The TM₅₀ value is the temperature at which heating for two minutes produces a 50% reduction in protein kinase activity (measured using any appropriate substrate) compared with the protein kinase activity before such heating, as described in Example 2. An increase in the TM₅₀ value, for example by at least about 1, 2, 3, 4, 5, 6, 7, 8, 9, 10 or 15° C. indicates stabilisation.

A further aspect of the invention provides a method of modulating in a cell the protein kinase activity of a protein kinase having a hydrophobic pocket in the position equivalent to the hydrophobic pocket of Protein Kinase A (PKA) that is defined by residues including Lys76, Leu116, Val80 and/or Lys111 of fill-length mouse PKA, wherein a recombinant interacting polypeptide is expressed in the cell, wherein the interacting polypeptide interacts with the hydrophobic pocket of the protein kinase and/or has the amino acid sequence Phe/Tyr-Xaa-Xaa-Phe/Tyr. Preferences for the protein kinase and interacting polypeptide are as indicated above. For example, GST-PIF may be expressed in a cell, as described in Example 1 and 2. The GST-PIF may inhibit the phosphorylation of p70 S6 kinase by PDK1.

Suitably, the method comprises the steps of providing a recombinant polynucleotide suitable for expressing the interacting polypeptide in the cell, providing the recombinant polynucleotide in the cell, and exposing the cell to conditions under which the cell expresses the interacting polypeptide from the recombinant polynucleotide.

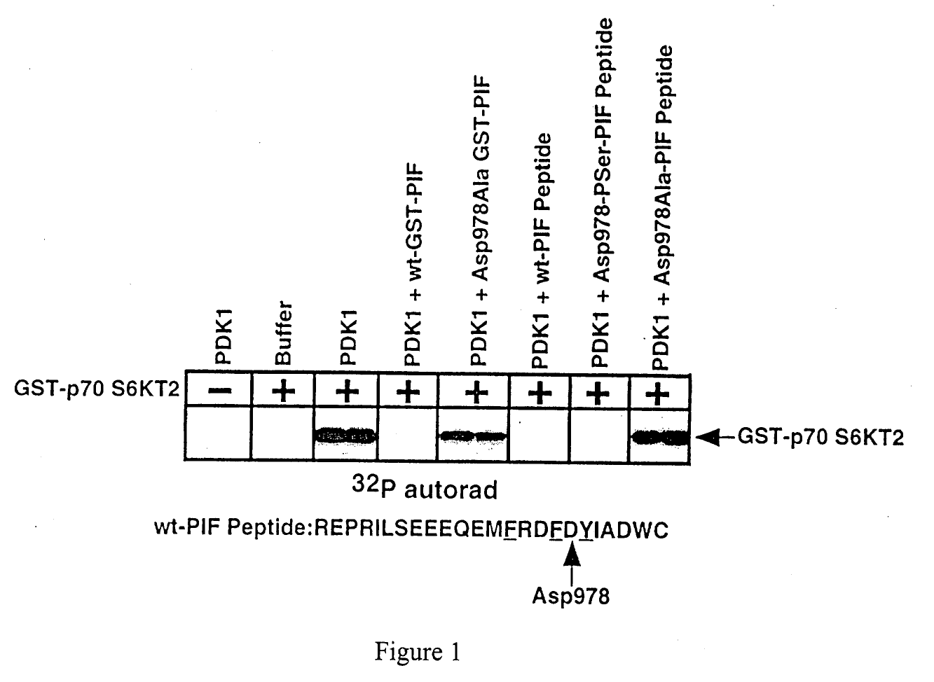

A further aspect of the invention provides a polypeptide which comprises the amino acid sequence Phe/Tyr-Xaa-Xaa-Phe/Tyr, wherein said polypeptide does not comprise the amino acid sequence Phe/Tyr-Xaa-Xaa-Phe/Tyr-Zaa-Phe/Tyr or Phe/Tyr-Xaa-Xaa-Phe/Tyr-Ser/Thr-Phe/Tyr and is not full-length PKA. The polypeptide may have the C-terminal sequence Phe-Xaa-Xaa-Phe-COOH, or Phe/Tyr-Xaa-Xaa-Phe/Tyr-(X)ₙ—COOH, preferably Phe-Xaa-Xaa-Phe-(X)ₙ—COOH, wherein n is between 1 and 200, 150, 100, 50, 30, 20, 15, 10, 5, 4, 3 or 2, preferably between 1 and 4, most preferably 4. Each amino acid X is any amino acid residue, preferably glycine. Thus, it is preferred that the polypeptide has the C-terminal sequence Phe-Xaa-Xaa-Phe-COOH or Phe-Xaa-Xaa-Phe-(Gly)₄-COOH. The polypeptide may consist of or comprise contiguous residues derivable from PKA. For example, it may comprise the C-terminal about 223, 220, 200, 180, 160, 140, 120, 100, 80, 70, 65, 63, 55, 50, 45, 40, 35, 30, 25, 20, 15, 10 or 5 amino acids of PKA, or a variant or fusion thereof that has the C-terminal sequence Phe-Xaa-Xaa-Phe-COOH or Phe/Tyr-Xaa-Xaa-Phe/Tyr-(X)ₙ—COOH. PKA sequences are shown in FIGS. 15 and 16 and in the database records indicated in FIG. 1. The sequence of PKAα, for example, is also shown in Maldonado et al (1988) Nucl Acids Res 16(16), 8189-8190 (Accession no 4506055). Thus, for example, the polypeptide may comprise or consist essentially of the C-terminal 223 or 63 amino acids of PKA, for example human or mouse PKA. The polypeptide may be useful as an interacting polypeptide, as defined above.

The said polypeptide of the invention may comprise a GST portion, as described in Examples 1 and 2. This may be useful in purifying and/or detecting the said polypeptide.

A further aspect of the invention provides a polynucleotide encoding a polypeptide or mutated protein kinase of the invention. A still further aspect of the invention provides a recombinant polynucleotide suitable for expressing a polypeptide or mutated protein kinase of the invention. A yet further aspect of the invention provides a host cell comprising a polynucleotide of the invention.

A further aspect of the invention provides a method of making a polypeptide or mutated protein kinase of the invention, the method comprising culturing a host cell of the invention which expresses said polypeptide or mutated protein kinase and isolating said polypeptide or mutated protein kinase. It will be appreciated that the said polypeptide of the invention that comprises the amino acid sequence Phe/Tyr-Xaa-Xaa-Phe/Tyr, may be isolated as a complex with an endogenous protein kinase having a hydrophobic pocket in the position equivalent to the hydrophobic pocket of Protein Kinase A (PKA) that is defined by residues including Lys76, Leu116, Val80 and/or Lys111 of full-length mouse PKA, for example PDK1 expressed in the cell or with a recombinant said protein kinase expressed in the cell.

A further aspect of the invention provides a polypeptide or mutated protein kinase obtainable by the above method.

The interacting polypeptide as defined above may have up to about 950, 900, 800, 700, 600, 500, 400, 300, 200, 100, 80, 70, 60, 50, 40, 30, 20, 18, 16, 15, 14, 12, 10, 8 or 7 amino acids residues. It will be appreciated that the polypeptide may comprise a covalent modification, for example it may be modified by biotinylation ie comprise a biotin group. Such a peptide may be useful in the methods of the invention, for example in altering the enzymic activity of a protein kinase, for example PDK1 in vitro or in vivo.

The above polypeptides may be made by methods well known in the art and as described below and in Example 1 or 2, for example using molecular biology methods or automated chemical peptide synthesis methods.

It will be appreciated that peptidomimetic compounds may also be useful. Thus, by “polypeptide” or “peptide” we include not only molecules in which amino acid residues are joined by peptide (—CO—NH—) linkages but also molecules in which the peptide bond is reversed. Such retro-inverso peptidomimetics may be made using methods known in the art, for example such as those described in Mézière et al (1997) J. Immunol. 159, 3230-3237, incorporated herein by reference. This approach involves making pseudopeptides containing changes involving the backbone, and not the orientation of side chains. Meziere et al (1997) show that, at least for MHC class II and T helper cell responses, these pseudopeptides are useful. Retro-inverse peptides, which contain NH—CO bonds instead of CO—NH peptide bonds, are much more resistant to proteolysis.

Similarly, the peptide bond may be dispensed with altogether provided that an appropriate linker moiety which retains the spacing between the Cα atoms of the amino acid residues is used; it is particularly preferred if the linker moiety has substantially the same charge distribution and substantially the same planarity as a peptide bond.

It will be appreciated that the peptide may conveniently be blocked at its N- or C-terminus so as to help reduce susceptibility to exoproteolytic digestion.

Thus, it will be appreciated that the interacting polypeptide, for example which comprises the amino acid sequence Phe/Tyr-Xaa-Xaa-Phe/Tyr to which a protein kinase having a hydrophobic pocket in the position equivalent to the hydrophobic pocket of Protein Kinase A (PKA) that is defined by residues including Lys76, Leu116, Val80 and/or Lys111 of full-length mouse PKA, may be exposed may be a peptidomimetic compound, as described above.

A further aspect of the invention is a cell containing a recombinant nucleic acid suitable for expressing a protein kinase having a hydrophobic pocket in the position equivalent to the hydrophobic pocket of Protein Kinase A (PKA) that is defined by residues including Lys76, Leu116, Val80 and/or Lys111 of full-length mouse PKA, and a recombinant nucleic acid suitable for expressing a second polypeptide comprising the amino acid sequence Phe/Tyr-Xaa-Xaa-Phe/Tyr, wherein when the said protein kinase is PDK1, the said second polypeptide is not PIF, as defined above, and does not comprise the amino acid sequence Phe/Tyr-Xaa-Xaa-Phe/Tyr-Ser/Thr-Phe/Tyr. Recombinant polynucleotides suitable for expressing a given polypeptide are well known to those skilled in the art, and examples are described in Examples 1 and 2. It will be appreciated that a recombinant nucleic acid molecule may be suitable for expressing the protein kinase and the second polypeptide comprising the amino acid sequence Phe/Tyr-Xaa-Xaa-Phe/Tyr. The cell is preferably a mammalian or insect cell.

A further aspect of the invention is a method of making a preparation comprising a protein kinase having a hydrophobic pocket in the position equivalent to the hydrophobic pocket of Protein Kinase A (PKA) that is defined by residues including Lys76, Leu116, Val80 and/or Lys111 of full-length mouse PKA, and a second, interacting compound, wherein the interacting compound interacts with the hydrophobic pocket of the protein kinase, wherein the said preparation does not comprise all of the components found in a cell in which said protein kinase or compound is naturally found, and wherein when the protein kinase is PDK1, the interacting compound is not a polypeptide comprising the amino acid sequence Phe/Tyr-Xaa-Xaa-Phe/Tyr-Zaa-Phe/Tyr, wherein the said protein kinase and the said interacting polypeptide are co-expressed in a cell as defined in the above aspect of the invention. The protein kinase and the interacting polypeptide may be separated from other cellular components, for example using methods discussed above or in Examples 1 and 2. A further aspect of the invention is a preparation obtainable by the above method of the invention.

An antibody reactive towards p70 S6 kinase or a fragment or fusion thereof that is phosphorylated on the residue equivalent to Thr412 of the longer splice variant of human α-isoform of p70 S6 kinase, but is not reactive with p70 S6 kinase or a fragment or fusion thereof that is not phosphorylated on the said residue equivalent to Thr412, is described in Example 1 and is available from Upstate Biotechnology Inc., 199 Saranac Avenue, Lake Placed, N.Y., USA. A similar antibody is available from New England Biolabs (UK) Ltd, Knowl Piece, Wilbury Way, Hitchin, Herts, SG4 0TY. The antibody may react with the peptide SESANQVFLGFTYVAPSV (corresponding to residues 401 to 418 of the said longer splice variant) in which the underlined residue is phosphorothreonine. Methods of preparing such an antibody are given in Example 1.

Antibodies reactive towards the said polypeptides may be made by methods well known in the art. In particular, the antibodies may be polyclonal or monoclonal.

Suitable monoclonal antibodies which are reactive towards the said polypeptide may be prepared by known techniques, for example those disclosed in “Monoclonal Antibodies: A manual of techniques”, H Zola (CRC Press, 1988) and in “Monoclonal Hybridoma Antibodies: Techniques and Applications”, SGR Hurrell (CRC Press, 1982).

Techniques for preparing antibodies are well known to those skilled in the art, for example as described in Harlow, ED & Lane, D “Antibodies: a laboratory manual” (1988) New York Cold Spring Harbor Laboratory.

It will be appreciated that the invention provides screening assays for drugs which may be useful in modulating, for example either enhancing or inhibiting, the protein kinase activity of a protein kinase (for example, the protein kinase activity towards a particular substrate) having a hydrophobic pocket in the position equivalent to the hydrophobic pocket of Protein Kinase A (PKA) that is defined by residues including Lys76, Leu116, Val80 and/or Lys111 of full-length mouse PKA, for example PDK1, SGK, PKB, PKA or p70 S6 kinase, for example the PDK1 or PDK2 activity (as discussed above) of PDK1. The compounds identified in the methods may themselves be useful as a drug or they may represent lead compounds for the design and synthesis of more efficacious compounds.

The compound may be a drug-like compound or lead compound for the development of a drug-like compound for each of the above methods of identifying a compound. It will be appreciated that the said methods may be useful as screening assays in the development of pharmaceutical compounds or drugs, as well known to those skilled in the art.

The term “drug-like compound” is well known to those skilled in the art, and may include the meaning of a compound that has characteristics that may make it suitable for use in medicine, for example as the active ingredient in a medicament. Thus, for example, a drug-like compound may be a molecule that may be synthesised by the techniques of organic chemistry, less preferably by techniques of molecular biology or biochemistry, and is preferably a small molecule, which may be of less than 5000 daltons. A drug-like compound may additionally exhibit features of selective interaction with a particular protein or proteins and be bioavailable and/or able to penetrate cellular membranes, but it will be appreciated that these features are not essential.

The term “lead compound” is similarly well known to those skilled in the art, and may include the meaning that the compound, whilst not itself suitable for use as a drug (for example because it is only weakly potent against its intended target, non-selective in its action, unstable, difficult to synthesise or has poor bioavailability) may provide a starting-point for the design of other compounds that may have more desirable characteristics.

A further aspect of the invention is a kit of parts useful in carrying out a method, for example a screening method, of the invention. Such a kit may comprise a protein kinase having a hydrophobic pocket in the position equivalent to the hydrophobic pocket of Protein Kinase A (PKA) that is defined by residues including Lys76, Leu116, Val80 and/or Lys111 of full-length mouse PKA, for example PDK1, SGK, PKB, PKA or p70 S6 kinase, and an interacting polypeptide, for example a polypeptide comprising the amino acid sequence Phe/Tyr-Xaa-Xaa-Phe/Tyr and not comprising the amino acid sequence Phe/Tyr-Xaa-Xaa-Phe/Tyr-Zaa-Phe/Tyr or Phe/Tyr-Xaa-Xaa-Phe/Tyr-Ser/Thr-Phe/Tyr. It may further comprise a substrate polypeptide, as defined above. Preferences for the protein kinase, substrate polypeptide and interacting polypeptide are as indicated above. The kit may comprise a mutated protein kinase of the invention.

It will be understood that it will be desirable to identify compounds that may modulate the activity of the protein kinase in vivo. Thus it will be understood that reagents and conditions used in the method may be chosen such that the interactions between, for example, the said protein kinase and the interacting polypeptide, are substantially the same as between the human protein kinase and a naturally occuring interacting polypeptide comprising the said amino acid sequence. It will be appreciated that the compound may bind to the protein kinase, or may bind to the interacting polypeptide.

The compounds that are tested in the screening methods of the assay or in other assays in which the ability of a compound to modulate the protein kinase activity of a protein kinase, for example a hydrophobic pocket-containing protein kinase, as defined above, may be measured, may be compounds that have been selected and/or designed (including modified) using molecular modelling techniques, for example using computer techniques.

A further aspect of the invention provides a method of selecting or designing a compound that modulates the activity of a hydrophobic pocket-containing protein kinase as defined above, the method comprising the step of using molecular modelling means to select or design a compound that is predicted to interact with the said hydrophobic pocket-containing protein kinase, wherein a three-dimensional structure of a compound is compared with a three-dimensional structure of the said hydrophobic pocket and/or with a three-dimensional structure of an interacting polypeptide, as defined above, and a compound that is predicted to interact with the said hydrophobic pocket is selected.

Thus, the three-dimensional structure of a compound may be compared with the three-dimensional structure of an interacting polypeptide comprising the amino acid sequence Phe/Tyr-Xaa-Xaa-Phe/Tyr. In particular, the structure of the compound may be compared with the structure of the portion (the interacting portion) of the interacting polypeptide that interacts with the hydrophobic pocket, as discussed above and in Example 2, for example the Phe/Tyr-Xaa-Xaa-Phe/Tyr portion of the interacting polypeptide. A compound that mimics the structure of the interacting polypeptide, preferably the interacting portion of the polypeptide, still more preferably the features of the interacting portion that interact with residues of the protein kinase that define the hydrophobic pocket, ie residues equivalent to Lys76, Leu116, Val80 and/or Lys111 of full-length mouse PKA, may be selected.

The three-dimensional structure of a compound may be compared with the three-dimensional structure of the hydrophobic pocket. A compound that can interact with the hydrophobic pocket, in particular residues equivalent to Lys76, Leu116, Val80 and/or Lys111 of full-length mouse PKA, in a similar manner (for example similar separation and/or type of interaction ie hydrophobic or ionic, and/or similar cumulative energy of interaction) to an interacting polypeptide may be selected. Methods of assessing the interaction are well known to those skilled in the art.

The three-dimensional structures that are compared may be predicted three-dimensional structures or may be three-dimensional structures that have been determined, for example by techniques such as X-ray crystallography, as well known to those skilled in the art. The three-dimensional structures may be displayed by a computer in a two-dimensional form, for example on a computer screen. The comparison may be performed using such two-dimensional displays.

The following relate to molecular modelling techniques: Blundell et al (1996) Stucture-based drug design Nature 384, 23-26; Bohm (1996) Computational tools for structure-based ligand design

Prog Biophys Mol Biol 66(3), 197-210; Cohen et al (1990) J Med Chem 33, 883-894; Navia et al (1992) Curr Opin Struct Biol 2, 202-210.

The following computer programs, for example, may be useful in carrying out the method of this aspect of the invention: GRID (Goodford (1985) J Med Chem 28, 849-857; available from Oxford University, Oxford, UK); MCSS (Miranker et al (1991) Proteins: Structure, Function and Genetics 11, 29-34; available from Molecular Simulations, Burlington, Mass.); AUTODOCK (Goodsell et al (1990) Proteins: Structure, Function and Genetics 8, 195-202; available from Scripps Research Institute, La Jolla, Calif.); DOCK (Kuntz et al (1982) J Mol Biol 161, 269-288; available from the University of California, San Francisco, Calif.); LUDI (Bohm (1992) J Comp Aid Molec Design 6, 61-78; available from Biosym Technologies, San Diego, Calif.); LEGEND (Nishibata et al (1991) Tetrahedron 47, 8985; available from Molecular Simulations, Burlington, Mass.); LeapFrog (available from Tripos Associates, St Louis, Mo.); Gaussian 92, for example revision C (M J Frisch, Gaussian, Inc., Pittsburgh, Pa) ©1992); AMBER, version 4.0 (P A Kollman, University of California at San Francisco, ©1994); QUANTA/CHARMM (Molecular Simulations, Inc., Burlington, Mass. ©1994); and Insight II/Discover (Biosym Technologies Inc., San Diego, Calif. ©1994). Programs may be run on, for example, a Silicon Graphics™ workstation, Indigo²™ or IBM RISC/6000™ workstation model 550.

The selected or designed compound may be synthesised (if not already synthesised) and tested for its effect on the relevant hydrophobic pocket-containing protein kinase, for example its effect on the protein kinase activity. The compound may be tested in a screening method of the invention.

A further aspect of the invention is a compound identified or identifiable by the above selection/design method of the invention.

A still further aspect of the invention is a compound (or polypeptide or polynucleotide) of the invention for use in medicine.

The compound (or polypeptide or polynucleotide) may be administered in any suitable way, usually parenterally, for example intravenously, intraperitoneally or intravesically, in standard sterile, non-pyrogenic formulations of diluents and carriers. The compound (or polypeptide or polynucleotide) may also be administered topically, which may be of particular benefit for treatment of surface wounds. The compound (or polypeptide or polynucleotide) may also be administered in a localised manner, for example by injection. The compound may be useful as an antifungal (or other parasitic, pathogenic or potentially parasitic or pathogenic organism) agent.

A further aspect of the invention is the use of a compound (or polypeptide or polynucleotide) as defined above in the manufacture of a medicament for the treatment of a patient in need of modulation of signalling by a protein kinase having a hydrophobic pocket in the position equivalent to the hydrophobic pocket of Protein Kinase A (PKA) that is defined by residues including Lys76, Leu116, Val80 and/or Lys111 of full-length mouse PKA, for example PDK1, SGK, PKB or p70 S6 kinase, for example insulin signalling pathway and/or PDK1/PDK2/SGK/PKB/p70 S6 kinase/PRK2/PKC/PKA signalling. The patient may be in need of inhibition of a said hydrophobic pocket-containing kinase in an infecting organism, for example the patient may have a fungal infection for which treatment is required. The compound may inhibit the infecting organism's (for example fungul) hydrophobic pocket-containing protein kinase, but may not inhibit the patient's equivalent hydrophobic pocket-containing protein kinase.

A further aspect of the invention is a method of treating a patient in need of modulation of signalling by a protein kinase having a hydrophobic pocket in the position equivalent to the hydrophobic pocket of Protein Kinase A (PKA) that is defined by residues including Lys76, Leu116, Val80 and/or Lys111 of full-length mouse PKA, for example PDK1, SGK, PKB or p70 S6 kinase, for example insulin signalling pathway and/or PDK1/PDK2/SGK/PKB/p70 S6 kinase/PRK2/PKC/PKA signalling, wherein the patient is administered an effective amount of a compound (or polypeptide or polynucleotide) as defined above.

A compound that is capable of reducing the activity of PKC, for example PKCβ, PRK1 or 2, PKA, PDK1 (ie the PDK1 and/or the PDK2 activity), PKB, SGK or p70 S6 kinase may be useful in treating cancer. PDK1, for example via PKB and/or SGK, may be capable of providing a survival signal that protects cells from apoptosis induced in a variety of ways (reviewed in Cross et at (1995) Nature 378, 785-789 and Alessi & Cohen (1998) Curr. Opin. Genetics. Develop. 8, 55-62). Thus, such compounds may aid apoptosis. Reduction of the activity of PDK1, PKB, SGK and/or p70 S6 kinase may promote apoptosis and may therefore be useful in treating cancer. Conditions in which aiding apoptosis may be of benefit may also include resolution of inflammation.

A compound is capable of increasing the activity of PDK1, PKB, SGK or p70 S6 kinase may be useful in treating diabetes or obesity, or may be useful in inhibiting apoptosis. Increased activity of PDK1, PKB, SGK or p70 S6 kinase may lead to increased levels of leptin, as discussed above, which may lead to weight loss; thus such compounds may lead to weight loss. For example, such compounds may suppress apoptosis, which may aid cell survival during or following cell damaging processes. It is believed that such compounds are useful in treating disease in which apoptosis is involved. Examples of such diseases include, but are not limited to, mechanical (including heat) tissue injury or ischaemic disease, for example stroke and myocardial infarction, neural injury and myocardial infarction. Thus the patient in need of modulation of the activity of PDK1, PKB, SGK or p70 S6 kinase may be a patient with cancer or with diabetes, or a patient in need of inhibition of apoptosis, for example a patient suffering from tissue injury or ischaemic injury, including stroke.

Thus, a further aspect of the invention provides a method of treating a patient with an ischaemic disease the method comprising administering to the patient an effective amount of a compound identified or identifiable by the screening methods of the invention.

A still further invention provides a use of a compound identifiable by the screening methods of the invention in the manufacture of a medicament for treating an ischaemic disease in a patient.

Thus, a further aspect of the invention provides a method of treating a patient with an ischaemic disease the method comprising administering to the patient an effective amount of a compound identifiable by the screening methods of the invention.

If the patient is a patient in need of promotion of apoptosis, for example a patient with cancer, it is preferred that the compound of the invention that is used in the preparation of the medicament is capable of reducing the activity of PDK1, PKB, SGK or p70 S6 kinase. If the patient is a patient with diabetes or a patient in need of inhibition of apoptosis, for example a patient with ischaemic disease, it is preferred that the compound of the invention that is used in the preparation of the medicament is capable of increasing the activity of PDK1, PKB, SGK or p70 S6 kinase.

The invention further provides a method of identifying a compound that modulates the protein kinase activity of a protein kinase having a hydrophobic pocket in the position equivalent to the hydrophobic pocket of mouse Protein Kinase A (PKA) that is defmed by residues including Lys76, Leu116, Val80 and/or Lys111 of full-length mouse PKA, for example PDK1, comprising the steps of (1) determining the effect of a test compound on the protein kinase activity of the said protein kinase, and/or a mutant thereof, and (2) selecting a compound capable of modulating the protein kinase activity of the said protein kinase to different extents towards (i) a substrate that binds to the said hydrophobic pocket of the said protein kinase (hydrophobic pocket-dependent substrate) and (ii) a substrate (such as PKB) that does not bind, or binds to a lesser extent than the first said substrate (hydrophobic pocket-independent substrate), to the said hydrophobic pocket of the said protein kinase.

It is preferred that the protein kinase is PDK1. Preferences indicated above apply to this and following aspects of the invention as appropriate.

A compound that inhibits the protein kinase activity of the said protein kinase (for example PDK1) to a greater extent towards the hydrophobic pocket-dependent substrate than towards the hydrophobic pocket-independent substrate may be selected.

When the protein kinase is PDK1, the hydrophobic pocket-dependent substrate may be SGK, PRK2, S6K1 or PKCζ. The hydrophobic pocket-independent substrate may be PKB.

A further aspect of the invention provides a method of identifying a compound that modulates the protein kinase activity of a protein kinase having a hydrophobic pocket in the position equivalent to the hydrophobic pocket of mouse Protein Kinase A (PKA) that is defined by residues including Lys76, Leu116, Val80 and/or Lys111 of full-length mouse PKA (for example PDK1), comprising the step of determining the effect of the compound on the protein kinase activity of, or ability of the compound to bind to (1) the said protein kinase mutated at a residue defining at least part of the said hydrophobic pocket of the protein kinase, for example the residue equivalent to lysine 76 of full-length mouse PKA.

The method may further comprise determining the effect of the compound on the protein kinase activity of, or ability of the compound to bind to, the protein kinase (for example PDK1) which is not mutated at the said residue defining at least part of the said hydrophobic pocket of the protein kinase. When the protein kinase is PDK1, it may lack a functional PH domain (ie it may lack a PH domain capable of binding a phosphoinositide).

The effect of the compound on the rate or degree of phosphorylation of a hydrophobic pocket-dependent substrate may be determined. A compound may be selected that decreases the protein kinase activity of the said protein kinase, for example PDK1, towards a hydrophobic pocket-dependent substrate and does not affect or increases the protein kinase activity towards a hydrophobic pocket-independent substrate, for example PKB when the kinase is PDK1. An activator of PDK1 may mimic insulin and may be useful in treating diabetes or obesity, and may protect cells from apoptosis.

A further aspect of the invention provides a kit of parts useful in carrying out a method according to the preceding aspect of the invention, comprising (1) a mutated protein kinase as defined above and/or the protein kinase which is not a mutated said protein kinase as defined above (2) a hydrophobic pocket-dependent substrate and a hydrophobic pocket-independent substrate of the said protein kinase.

A further aspect of the invention provides the use of a compound capable of inhibiting to a different extents the rate or degree of phosphorylation by a protein kinase having a hydrophobic pocket in the position equivalent to the hydrophobic pocket of mouse Protein Kinase A (PKA) that is defined by residues including Lys76, Leu116, Val80 and/or Lys111 of full-length mouse PKA (for example PDK1), of a hydrophobic pocket-dependent substrate than of a hydrophobic pocket-independent substrate of the protein kinase, in the manufacture of a medicament for the treatment of a patient in need of inhibition to different extents of (1) phosphorylation of a hydrophobic pocket-dependent substrate of the said protein kinase and (2) phosphorylation of a hydrophobic pocket-dependent substrate of the said protein kinase. Preferably the protein kinase is PDK1.

The compound may be an interacting polypeptide or compound as discussed above. For example, the compound may be PIF when the protein kinase is PDK1.

It is preferred that the compound inhibits to a greater degree the rate or degree of phosphorylation by the protein kinase (for example PDK1) of (1) a hydrophobic pocket-dependent substrate of the protein kinase than (2) a hydrophobic pocket-independent substrate of the protein kinase.

When the protein kinase is PDK1, the compound may be used to treat diabetes or cancer.

The invention will now be described by reference to the following Examples and Figures:

Figure Legends

FIG. 1. PIF prevents PDK1 from phosphorylating p70 S6K in vitro. GST-p70 S6K lacking the C-terminal 104 amino acids (GST-p70 S6KT2) (1 μg) was incubated for 30 min at 30° C. with Mg [γ³²P] ATP and GST-PDK1 (50 nM) in the presence or absence of either wild type (wt) GST-PIF or D978A GST-PIF (1.5 μM), Or the indicated PIF peptides (4 μM) in a final volume of 20 μl. The reactions were terminated by making the solutions 1% in SDS, the samples subjected to SDS-polyacylamide gel electrophoresis, and the phosphorylation assessed by autoradiography of the gel. The position in the gel where GST-p70 S6KT2 (73 kDa) migrates is indicated with an arrow. The only other ³²P-labelled protein on the gel which is not shown, corresponded to autophosphorylation of PDK1 and contained ˜10 fold lower levels of ³²P-radioactivity than that of the GST-p70 S6T2 phosphorylated by PDK1 in the absence of PIF. A high amount of PDK1 is used in this experiment to achieve a near maximal phosphorylation of GST-p70 S6T2. If the reactions were carried out at a 10-fold lower concentration of PDK1 under conditions where the phosphorylation of GST-p70 S6T2 by PDK1 is linear with time and the amount of substrate used, then PIF still prevented the phosphorylation of GST-p70 S6T2 (data not shown). wt indicates wild type and PSer indicates phospho-serine. The results of a duplicate experiment for each condition are shown, and similar findings were obtained in five separate experiments.

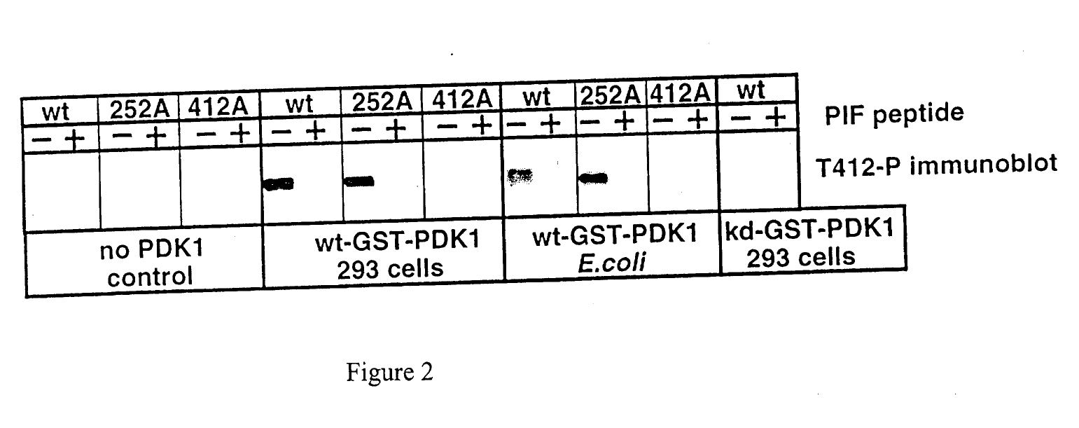

FIG. 2. PDK1 phosphorylates p70 S6K at Thr412 in vitro and this is inhibited by PIF. 0.5 μg of either wild type GST-p70 S6K-T2 (wt), T252A-GST-p70 S6K-T2 (252A) or T412A-GST-p70 S6K-T2 (412A) were incubated for 90 min at 30° C. with MgATP in the presence or absence of wild type (wt) or kinase-dead (kd) GST-PDK1 expressed in either 293 cells or bacteria in the presence (+) or absence (−) of the wild type PIF peptides (4 μM) in a final volume of 20 μl. The reactions were terminated by making the solutions 1% in SDS, the samples were subjected to SDS-polyacylamide gel electrophoresis, and the phosphorylation of p70 S6K at Thr412 was assessed by immunoblotting with the T-412P antibody. Similar results were obtained in three separate experiments.

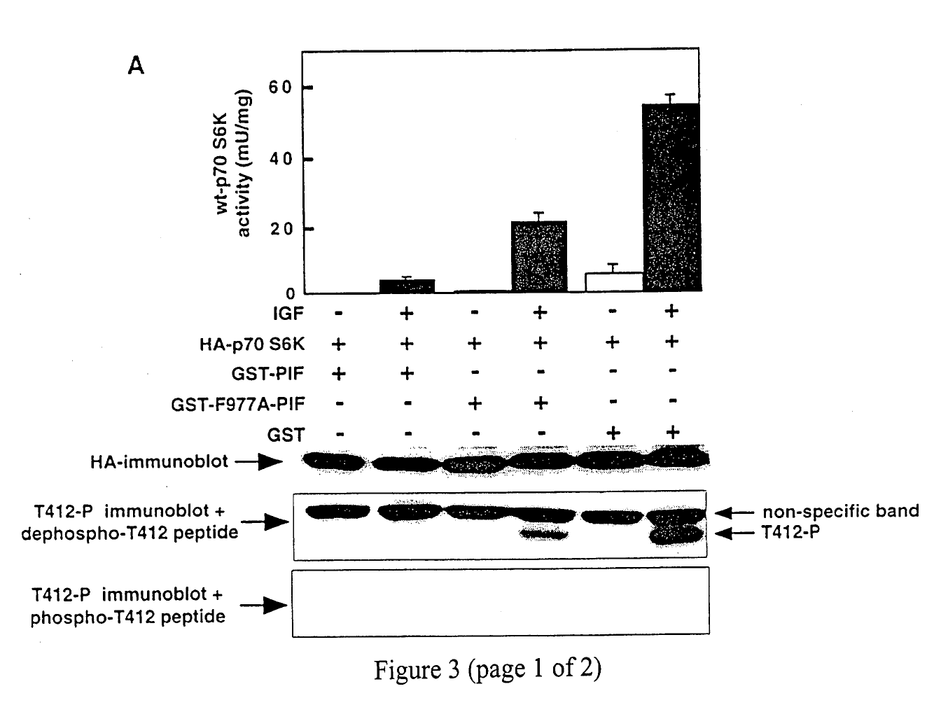

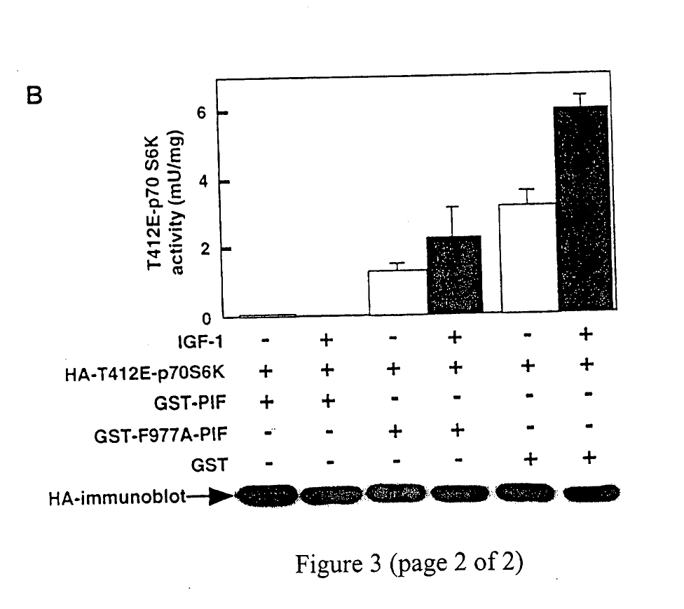

FIG. 3. PIF inhibits p70 S6K activation and phosphorylation at Thr252 and Thr412. 293 cells were co-transfected with constructs expressing the wild type (wt) full length HA-p70 S6K (A) or the full length HA-T412E p70 S6K (B) with either GST-PIF, GST-F977A-PIF, or GST. 24 h post transfection the cells were serum starved for 16 h and them stimulated for 40 min with 100 nM IGF1. The cells were lysed and HA-p70 S6K was immunoprecipitated and assayed as described in methods. Protein from each lysate (10 μg for the HA blots or 20 μg for the T412-P blot) was electrophoresed on a 10% SDS/polyacrylamide gel and immunoblotted using HA-antibody or the T412-P antibody. The T412-P antibody was incubated with either the synthetic peptide (10 μg/ml) corresponding to residues 401 to 418 of p70 S6K phosphorylated at Thr412 (phosph-412E peptide), or the unphosphorylated peptide (de-phospho-Thr412 peptide). The T412-P antibody consistenty recognises a protein termed “non specific band” in cell lysates which migrate at (75 kDa) derived from non transfected and tansfected cells. The intensity of this band does not change with IGF1. It is not co-immunoprecipitated with HA-p70 S6K (date not shown). The HA-p70 S6K activities shown are the average ±SEM for a single experiment carried out in triplicate. Similar results were obtained in 8 separate experiments (A) and 2 experiments (B). The immunoblotting was carried out in 3 separate experiments with similar results.

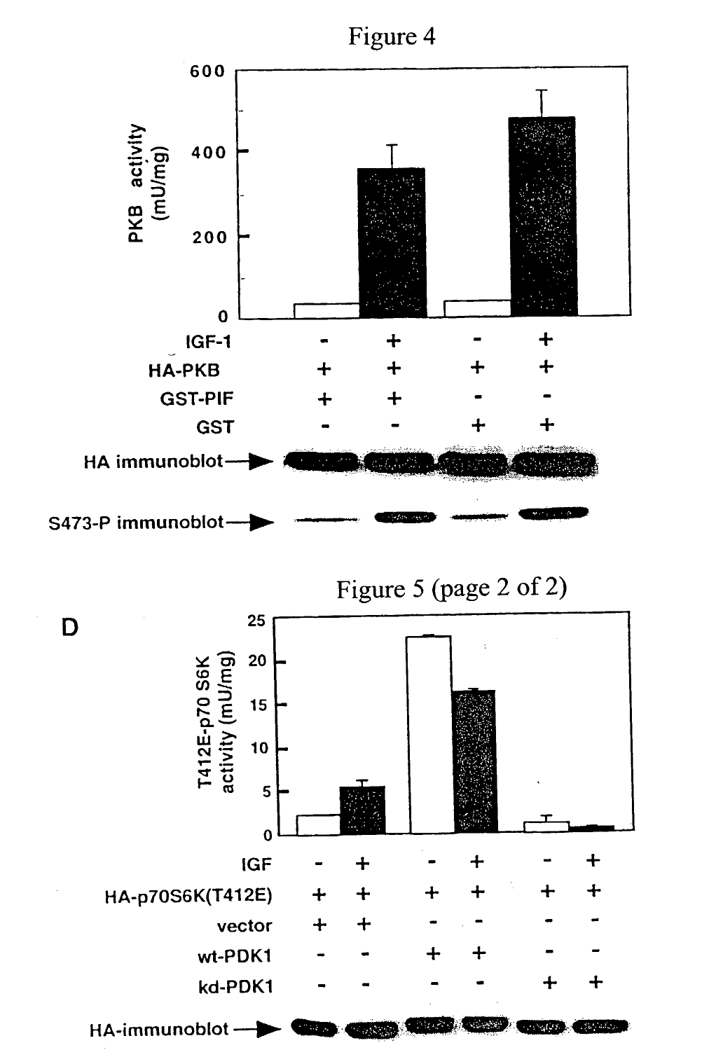

FIG. 4. PIF does not inhibit PKBα activation or its phosphorylation at Ser473. 293 cells were co-transfected with constructs expressing the wild type full length HA-PKBα with either GST-PIF or GST. 24 h post transfection the cells were serum starved for 16 h and then stimulated for 15 min with 100 nM IGF1. The cells were lysed and HA-PKBα was immunoprecipitated and assayed as described in Methods. Protein from each lysate (10 μg) was electrophoresed on a 10% SDS/polyarylamide gel and immunoblotted using HA-antibody or the S473-P antibody. The HA-PKBα activities shown are the averages ±SEM for a single experiment carried out in triplicate, similar results were obtained in 3 separate experiments.

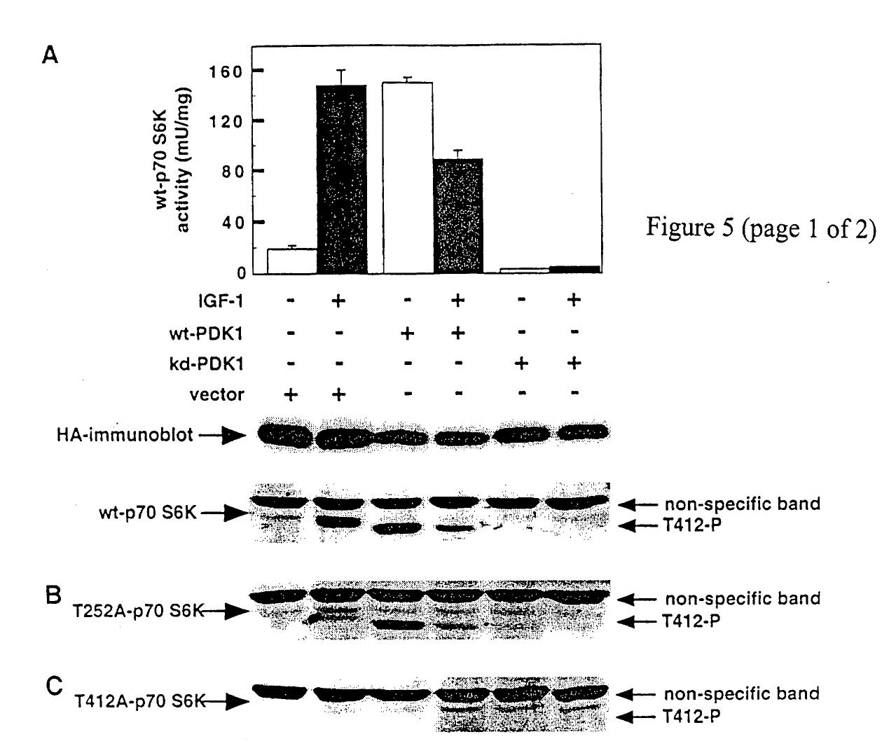

FIG. 5. A kinase-dead PDK1 inhibits p70 S6K activation and phosphorylation at Thr252 and Thr412. 293 cells were co-transfected with constructs expressing the wild type (wt) full length HA-p70 S6K (A) full length (A) full length HA-252A p70 S6K (B), full length HA-412A p70 S6K (C) or full length HA412E p70 S6K (D) with either wild type PDK1, a kinase-dead (kd) mutant or PDK1 or the empty pCMV5 vector. 24 h post transfection, the cells were serum starved for 16 h and then stimulated for 40 min with 100 nM IGF1. The cells were lysed, wild type and mutant forms of HA-p70 S6K immunoprecipitated and assayed as described in methods. The HA-252A p70 S6K or the HA-412A p70 S6K are essentially inactive under all conditions as reported previously [6] (data not shown). Protein from each lysate (10 μg for the HA blots or 20 μg for the T412-P blot) was electrophoresed on a 10% SDS/polyacrylamide gel and immunoblotted using HA-antibody or the T412-P antibody. The T412-P antibody was incubated in the presence of the dephosphorylated peptide corresponding to residues 401 to 418 of p70 S6K. The HA-p70 S6K activities shown are the average ±SEM for a single experiment carried out in triplicate. Similar results were obtained in at least 3 separate experiments. Comparable results to the HA and T412-P blots shown here also obtained in at least 3 separate experiments.

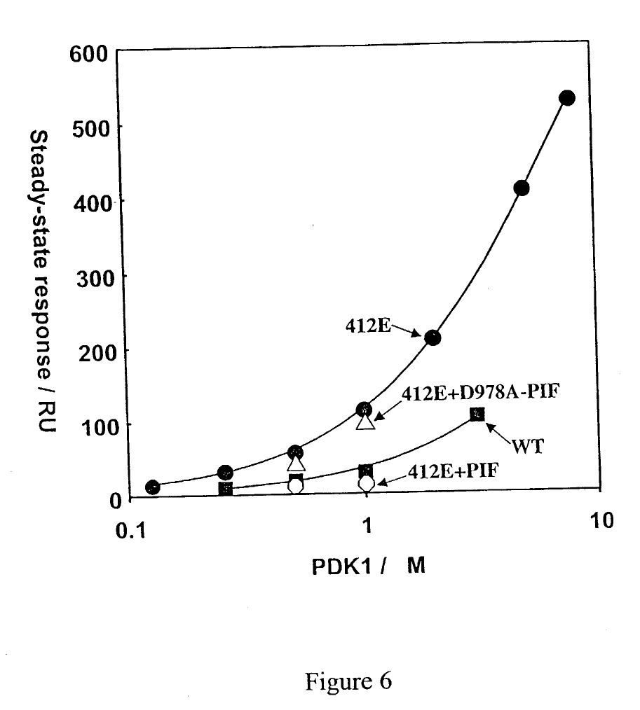

FIG. 6. Quantitative analysis of the binding of PDK1 to p70 S6K. Surface Plasmon Resonance measurements were carried out on a BiaCore instrument as described in the Methods. His-PDK1 was injected at the indicated concentrations over (A) 2000 RUs of p70 S6K-T2 (closed squares), or 412Ep70 S6K-T2 (closed circles) which was immobolised by amine coupling to a CM5 Sensorchip. Experiments carried out in the presence of either 10 μM wild type PIF peptide (hexagons) or 10 μM D978A mutant PIF peptide (triangles) are indicated. The responses at steady state binding were recorded. All data are single determinations from a representative experiment that was repeated at least 3 times with similar results. The data on the binding of wild type p70 S6K to PDK1 concentrations above 2 μM is not shown, as our analysis of the date suggested that non-specific protein binding was contributing to part of the observed binding response.

FIG. 7. Two hybrid interaction of PDK1 and wild type and mutant C-terminal fragment of PKA (A) The Y190 yeast strain was transformed with vectors expressing PDK1 fused to the Ga14 DNA binding domain (GBD), together with vectors encoding either PIF or the wild type or indicated mutants of a C-terminal fragment of PKA (PKA_{CT} residues 129-350) fused to a Ga14 activation domain (GAD). As a control, yeast were also co-transformed with the GBD domain alone and the GAD domain alone. The yeast were grown overnight at 30° C. and galactosidase filter lift assays performed at 30° C. for 4 h. An interaction between GBD-PDK1 and GAD-PKA_{CT} induces the expression of β-galactosidase which is detected as a blue colour in the filter lift assay. (B) Alignment of the amino acid sequence of the C-terminal 77 amino acids of PKA with the equivalent region of AGC subfamily kinases indicated. Identical residues are denoted by white letters on black background, and similar residues by grey boxes. The aromatic residues in the hydrophobic motif are indicated by arrows.

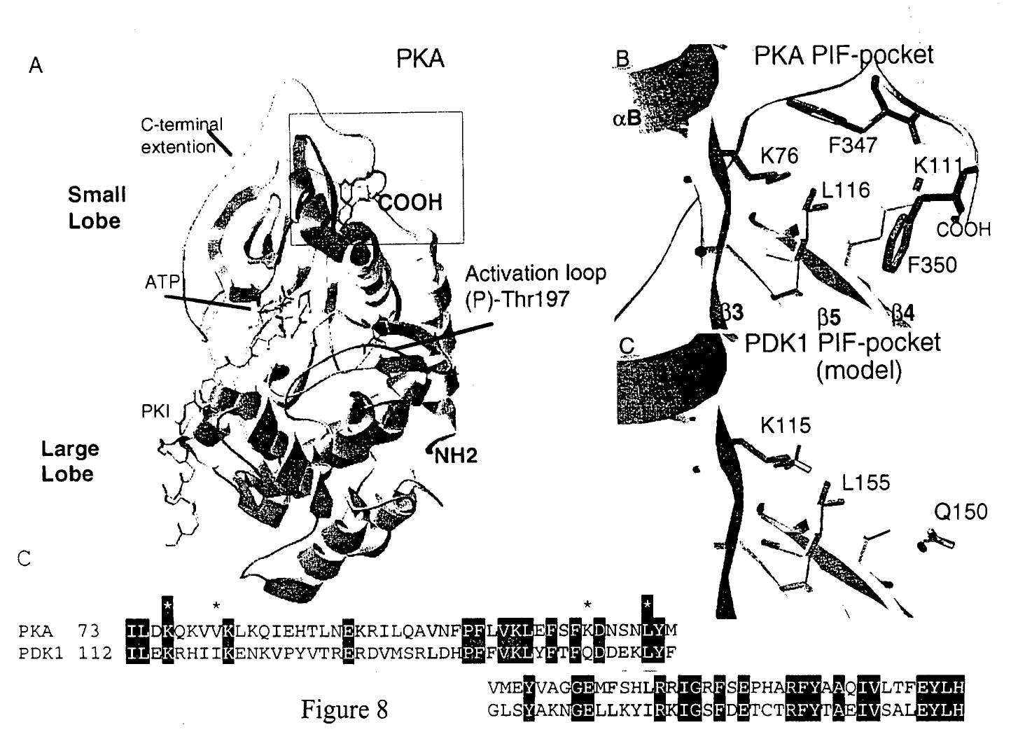

FIG. 8. C-terminal Phe-Xaa-Xaa-Phe residues of PKA interact with a hydrophobic pocket on the PKA kinase domain, predicted to be conserved in PDK1. (A) Ribbon structure of the PKA/PKI/ATP tenary complex [33: Example 2]; PKI and the ATP molecule are indicated. The C-terminal Phe 347 and Phe 350, are indicated. The position of phospho-Thr 197 (the PDK1 phosphorylation site) in the T-loop is indicated. (B) Detailed structure of the hydrophobic pocket on the kinase domain of PKA that interacts with the C-terminal Phe-Xaa-Xaa-Phe residues of PKA. Lys76 (equivalent of Lys115 in PDK1), Leu 116 (equivalent of Leu155 in PDK1), Phe347 and Phe350, and certain amine residues are shown. (C) The structure of the PDK1 kinase domain was modelled as described in methods. The region of PDK1 equivalent to the hydrophobic pocket of PKA termed the PIF-binding pocket is shown. Residues predicted to be involved in binding to PIF are highlighted. (D) Alignment of the amino acid residues of PDK1 around the PIF-binding pocket and the equivalent region of PKA. Identical residues are denoted by white letter on black background and similar residues by grey boxes. Residues on PKA which interact with the C-terminal Phe-Xaa-Xaa-Phe motif are marked with an asterisk.

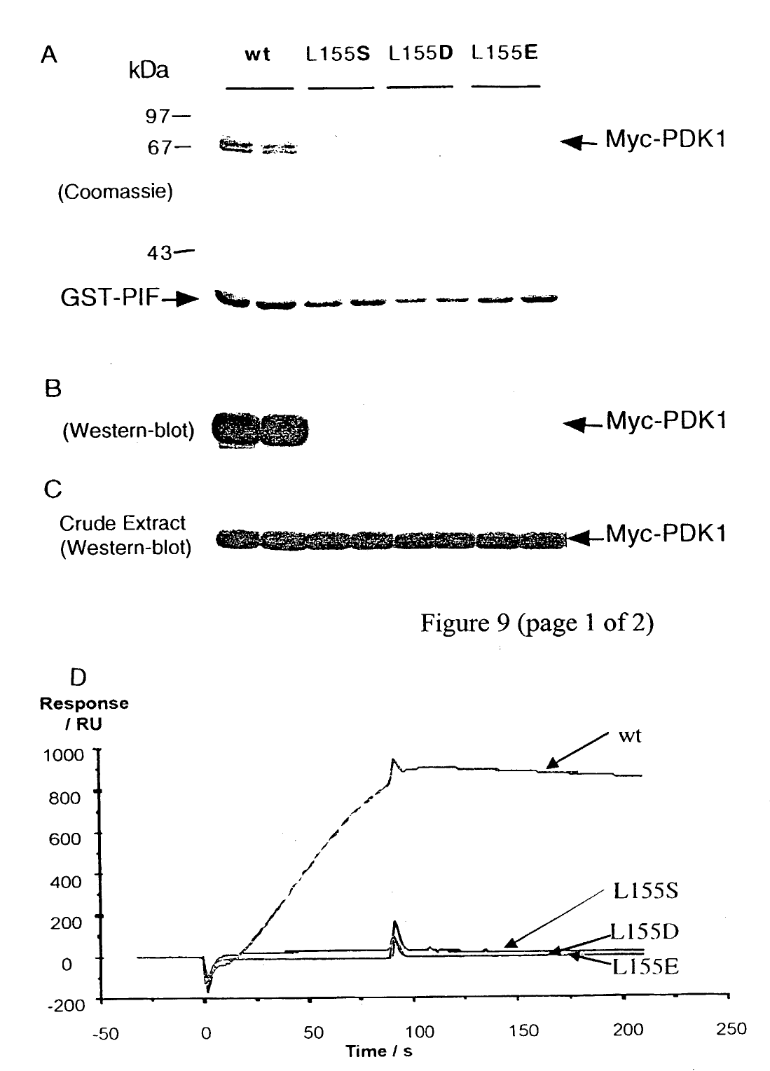

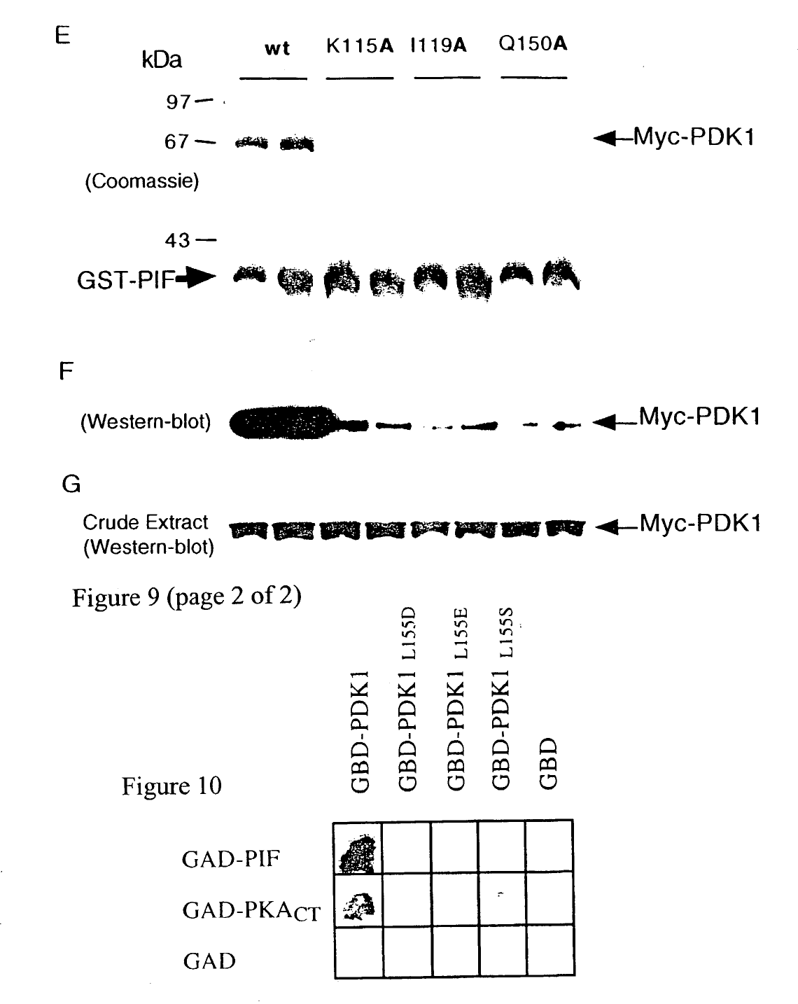

FIG. 9. Effect of mutation of conserved residues in the PIF-binding pocket of PDK1 on the ability to interact with PIF. 293 cells were transiently transfected with DNA constructs expressing GST-PIF and either wild type Myc-PDK1 or the indicated mutants of PDK1. 36 h post transfection the cells were lysed and GST-PIF purified by affinity chromatography on glutathione-Sepharose beads. 2 μg of each protein was electrophoresed on a 10% SDS/polyacrylamide gel and either stained with Coomassie blue (A and E) or immunoblotted using an anti Myc antibody to detect Myc-PDK1 (B and F). To establish that the wild type PDK1 and mutant proteins were expressed at a similar level, 10 μg of total cell lysate was electrophoresed on a 10% SDS/polyacrylamide gel and immunoblotted using anti-Myc antibodies (C and G). Duplicates of each condition are shown. Similar results were obtained in 3 to 5 separate experiments. (D) Surface Plasmon Resonance measurements were carried out in a BiaCore instrument as described in the Methods to measure the interaction of wild type and mutant GST-PDK1 preparations with the 24 residue synthetic peptide whose sequence encompasses the PDK1 binding site on PIF termed PIFtide [24: Example 2]. PIFtide was immobilised on a SA SensorChip and wild type (wt) or the indicated mutants of PDK1 were injected at a concentration of 40 nM. All data are single determinations from a representative experiment that was repeated at least 3 times with similar results. For clarity, the bulk, refractive index changes associated with the beginning and end 10 seconds of the injection have been removed.

FIG. 10. Leu155 mutants of PDK1 do not interact with either PIF or the C-terminal fragment of PKA in the two hybrid system. The Y190 yeast strain was transformed with vectors expressing the wild type PDK1 or the indicated mutants of PDK1 fused to the Ga14 DNA binding domain (GBD) together with vectors encoding for the expression of either the 77 C-terminal residues of PRK2 (PIF) or the C-terminal fragment of PKA (PKA_{CT} residues 129-350 fused to a Gal4 activation domain (GAD). As a control yeast were also co-transformed with vectors expressing the GAD and GBD domains only. The yeast were grown overnight at 30° C. and galactosidase filter lift assays performed at 30° for 4 h. An interaction between GBD-PDK1 and either GAD-PIF or GAD-PKA_{CT} induces the expression of β-galactosidase which is detected as a blue colour in the filter lift assay.

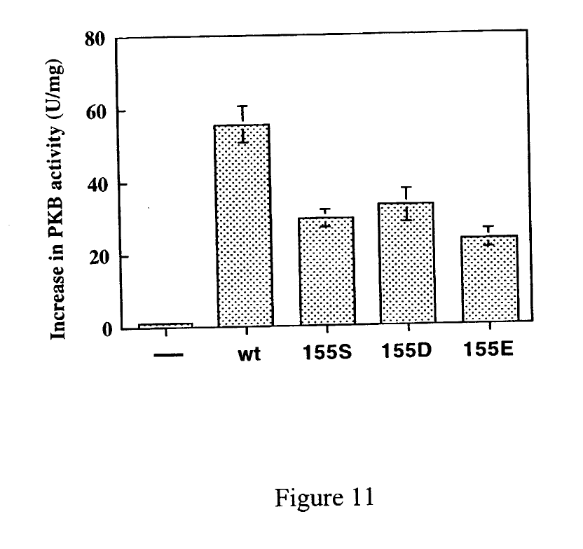

FIG. 11. Phosphorylation of Thr308 of PKB by wild type and PIF-binding pocket mutants of PDK1. Wild type or mutants forms of GST-PDK1 were expressed in 293 cells and purified by affinity chromatography on glutathione-Sepharose beads. Each GST-fusion protein (0.2 ng) was incubated for 30 min at 30° C. with GST-S473D-PKBα and MgATP in the presence or absence of phospholipid vesicles containing 100 μM phosphatidycholine. 100 μM phosphatidyylserine and 10 μM sn-1-stearoyl-2-arachidonoyl-D-PtdIns (3, 4, 5) P₃ and the increase in specific activity of GST-S473D-PKBα was determined relative to a control incubation in which the PDK1 was omitted (average for 6 determinations, three independent experiments). The basal activity of GST-S473D-PKBα was 1.5 U/mg. Under the conditions used it was verified that the activation of GST473D-PKBα was proportional to the amount of PDK1 added to the assay (data not shown). (-indicates PDK1 was omitted.

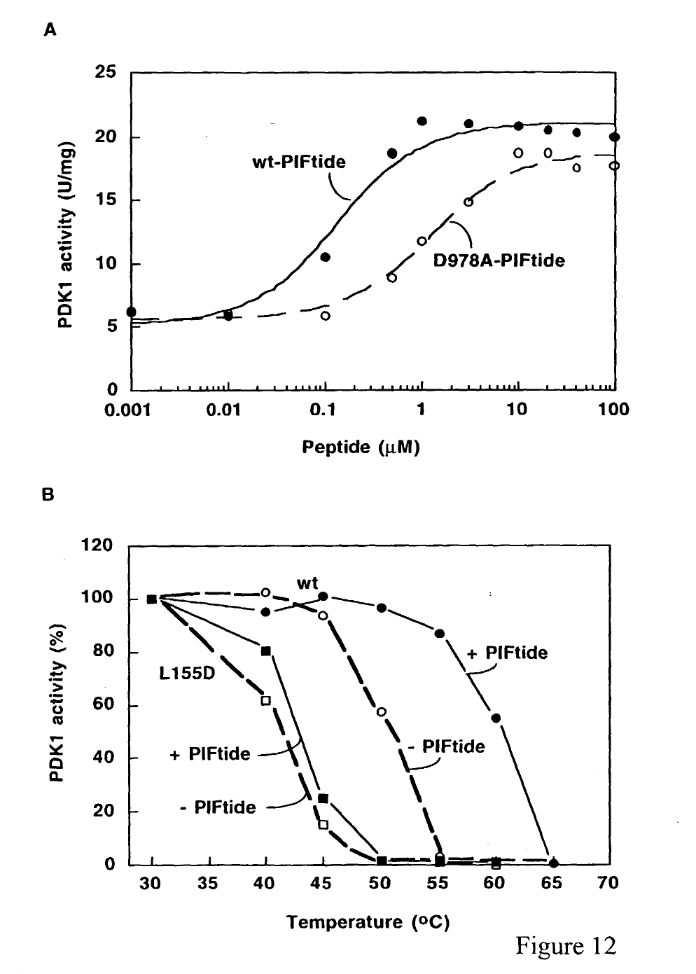

FIG. 12. PDK1 is activated and stabilised through its interaction with PIFtide. (A) GST-PDK1 activity was measured in the presence of increasing concentrations of wild type (wt) PIFtide (closed circles) or a mutant D978A PIFtide (open circles) using the synthetic peptide substrate termed T308tide, as described in Material and Methods. The data was fitted to a hyperbola using the Kaleidagraph™ software. The connection needed to obtain 50% activation of PDK1 was 0.14 μM for wt-PIFtide and 1.1 μM for D978A-PIFtide. The assay shown was performed in triplicate and there was less than 5% difference between each assay. Similar results were obtained in 2 further experiments (B)—The wild type GST-PDK1 (circles) or the L155D mutant of GST-PDK1 (squares) was incubated in the presence (closed symbols) or absence (open symbols) of 100 μM PIFtife and then for 2 min at the indicated temperatures, rapidly brought to 0° C. (see Materials and Methods), and 2 min later assayed at 30° C. for 10 min using T308tide as substrate. The activity of PDK1 obtained by incubation at 30° C. was taken as 100%. The assay shown was performed in duplicate with similar results obtained in two separate experiments.

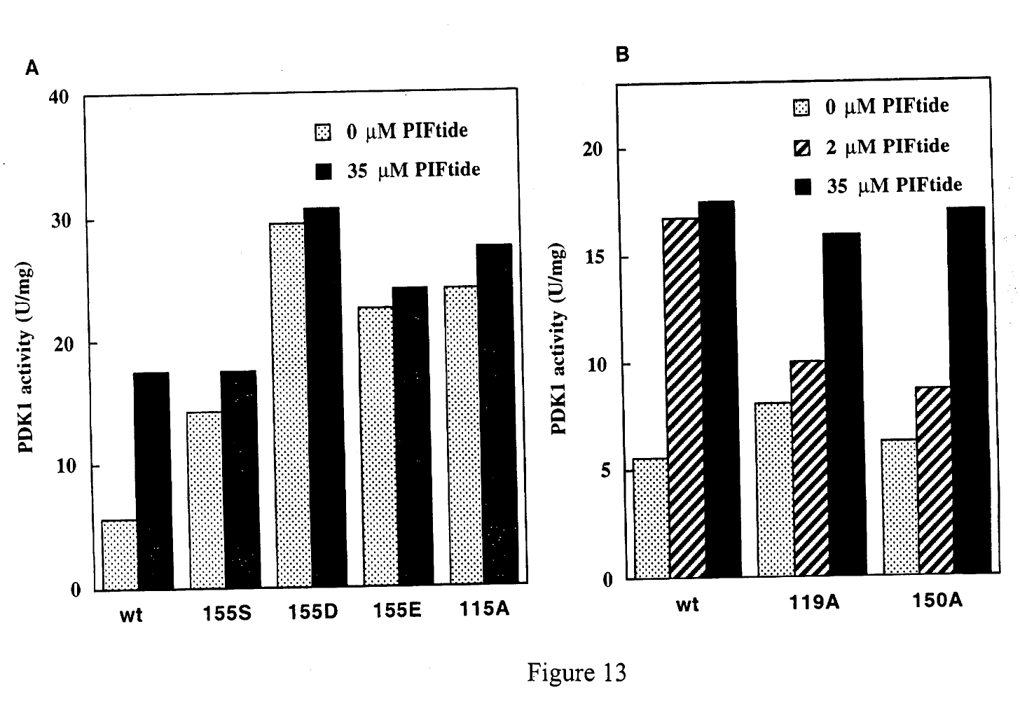

FIG. 13. Effect of PIFtide on PDK1 pocket mutants. Wild type and the indicated mutants of GST-PDK1 were assayed with T308tide either in the absence (dotted bars) or in the presence of 2 mM PIFtide (dashed bars), or 35 μM PIFtide (filled bars). Under the conditions used the phosphorylation of T308tide by PDK1 was linear with time (data not shown). (A) shows the PDK1 mutants which are activated in the absence of PIFtide, and (B) the mutants which are activated by high concentrations of PIFtide. The assay was performed in triplicate with less than 10% difference between triplicate samples. Similar results were obtained in 3 separate experiments.

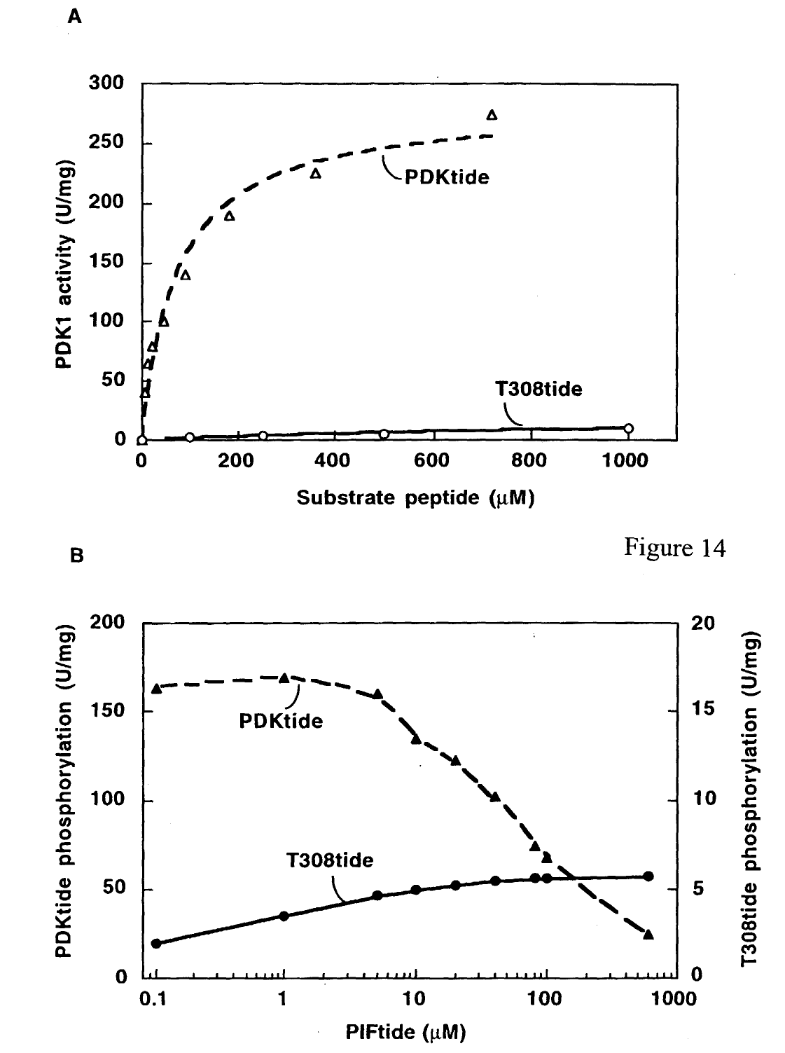

FIG. 14. PDKtide is a vastly superior substrate for PDK1 than T308tide because it interacts with the PIF-binding pocket of PDK1. (A) His-PDK1 was assayed for activity using as substrate the indicated concentration of either PDKtide (open triangles) or T308tide (open circles). (B) HIS-PDK1 was assayed for activity in the presence if PDKtide (25 μM closed triangles) or T308tide (100 μM closed circles) in the presence of the indicated concentrations of PIFtide. The assay was performed in triplicates with less than 5% difference between the triplicate samples. Similar results were obtained in 3 separate experiments.

FIG. 15. Alignment of AGC protein kinase family members. The residue equivalent to Lys 76 of mouse PKA (or residue Lys77 of human PKAα) is indicated. The residues equivalent to Val80, Lys111 and Leu116 of mouse PKA are also indicated. The position of the hydrophobic motif Phe/Tyr-Xaa-Xaa-Phe/Tyr is indicated by double lines.

FIG. 16. Alignment of further AGC protein kinase family members. The residue equivalent to Lys 76 of mouse PKA (or residue Lys77 of human PKAα) is indicated. The residues equivalent to Val80, Lys111 and Leu116 of mouse PKA are also indicated. The position of the hydrophobic motif Phe/Tyr-Xaa-Xaa-Phe/Tyr is indicated by double lines.

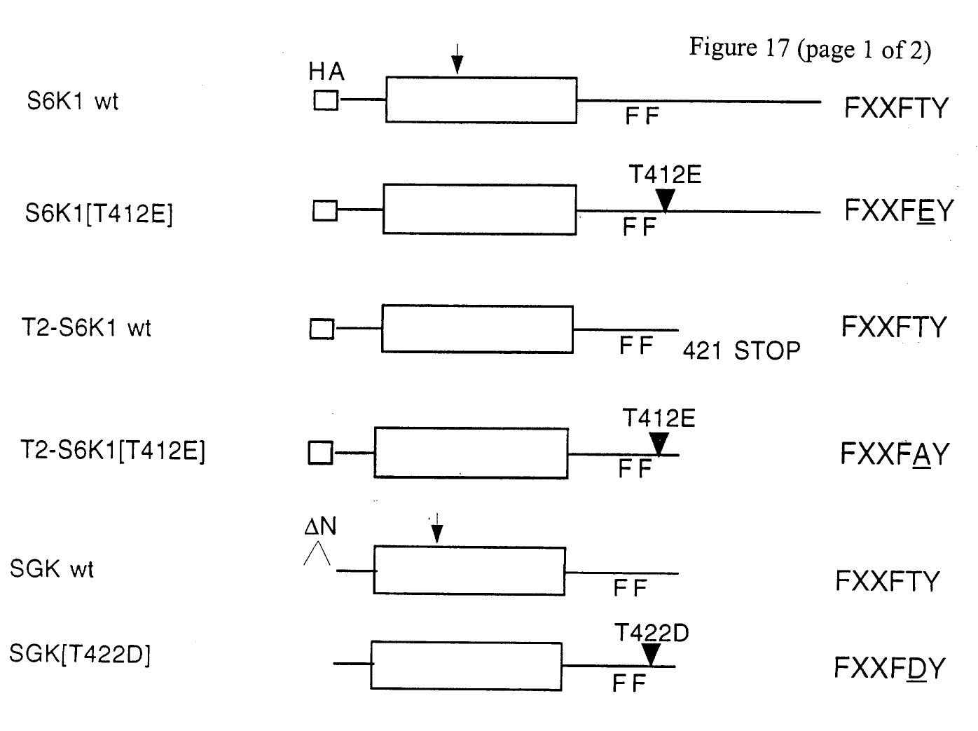

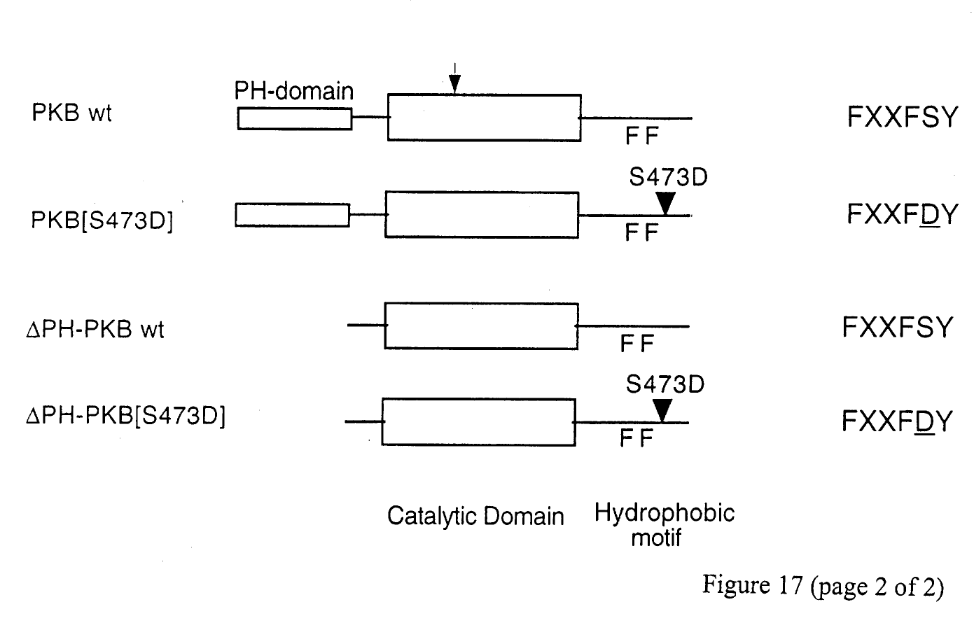

FIG. 17. Protein used a substrates of PDK1.

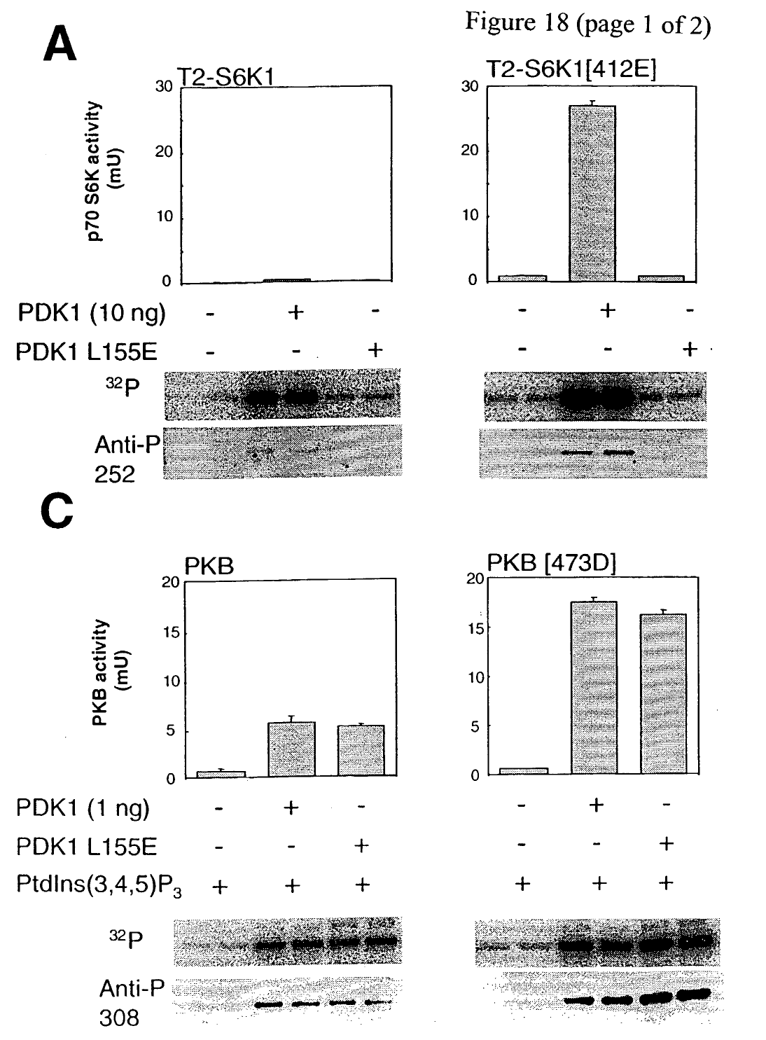

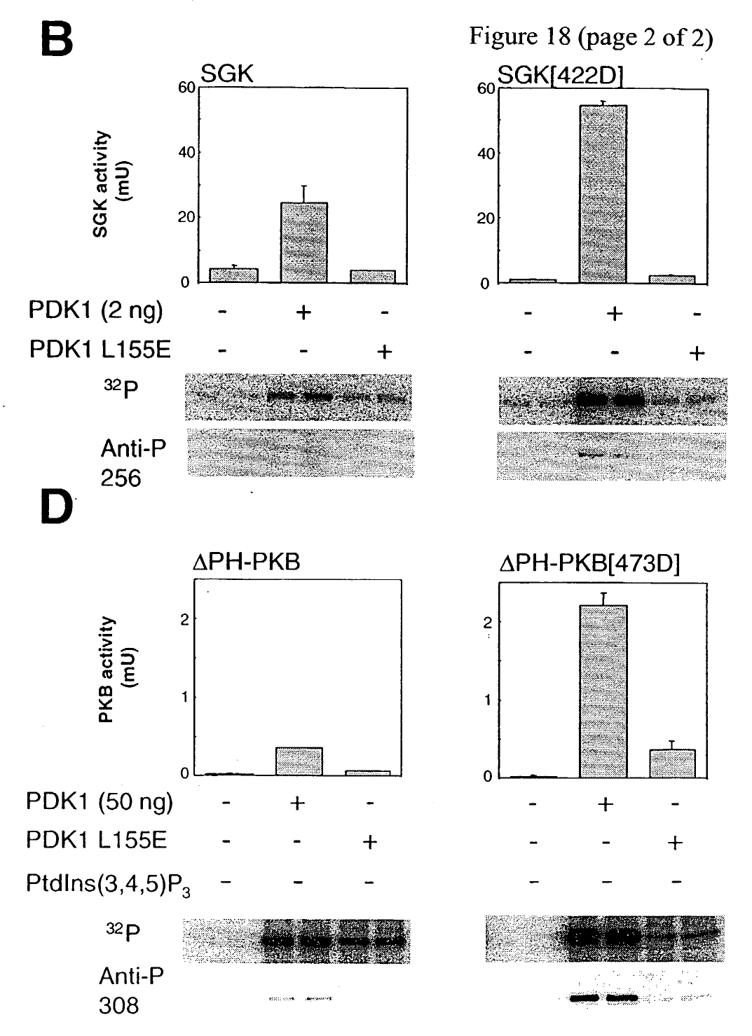

FIG. 18. Phosphorylation and activation of substrates by PDK1 PIF pocket mutant Leu155Glu. GST-PDK1 and GST-PDK1 L155E were tested for their ability to phosporylate and activate the different substrates. PDK1 L155E is known to disrupt the hydrophobic PIF pocket. Substrates (0.6 μM) were incubated in vitro in the absence or in the presence of PDK1 or PDK1 L155E. Activation of substrates was assessed by further incubating the reaction mixture with [γ-³²P]ATP and the peptide substrate Crosstide. Activation of the substrate protein kinase is observed as a difference between the activity without or in the presence of the stated concentration of PDK1. Phosphorylation of the substrates was quantified by performing the phosphorylation reaction in the presence of [γ-³²P]ATP, separating the products of the reaction by SDS-PAGE followed by phosphoimager analysis. Parallel experiments were blotted with antibodies that specifically detect the phosphorylated form of the 256 site on S6K1, 252 site on SGK1 and 308 site on PKB. Immunoblots to detect the phosphorylated form of the hydrophobic motif site of S6K1 and PKB under these conditions did not reveal any band (not shown). Under the conditions used, phosphorylation of substrates by PDK1 were linear with time and amount of enzyme. Experiments were performed in duplicates at least two times. The results shown correspond to one particular experiment. Duplicates within one experiment did not vary more than 10%, usually less than 5%. Substrates tested were (A) Baculovirus expressed His-tag S6K1 T2 and S6K1 T2 412E, (B) GST-SGK1 and GST-SGK1 422D previously dephosphorylated with PP2A, (C) GST-FL-PKB and GST-FL-PKB 473D, (D) GST-PKB-ΔPH and GST-PKB-ΔPH.

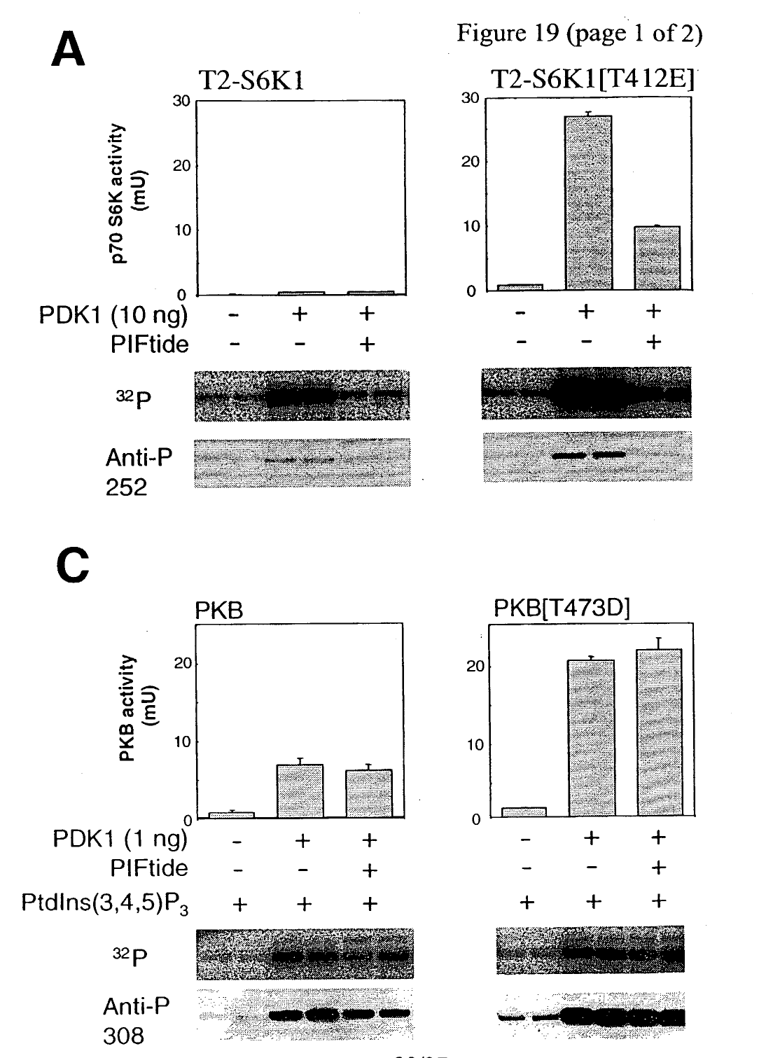

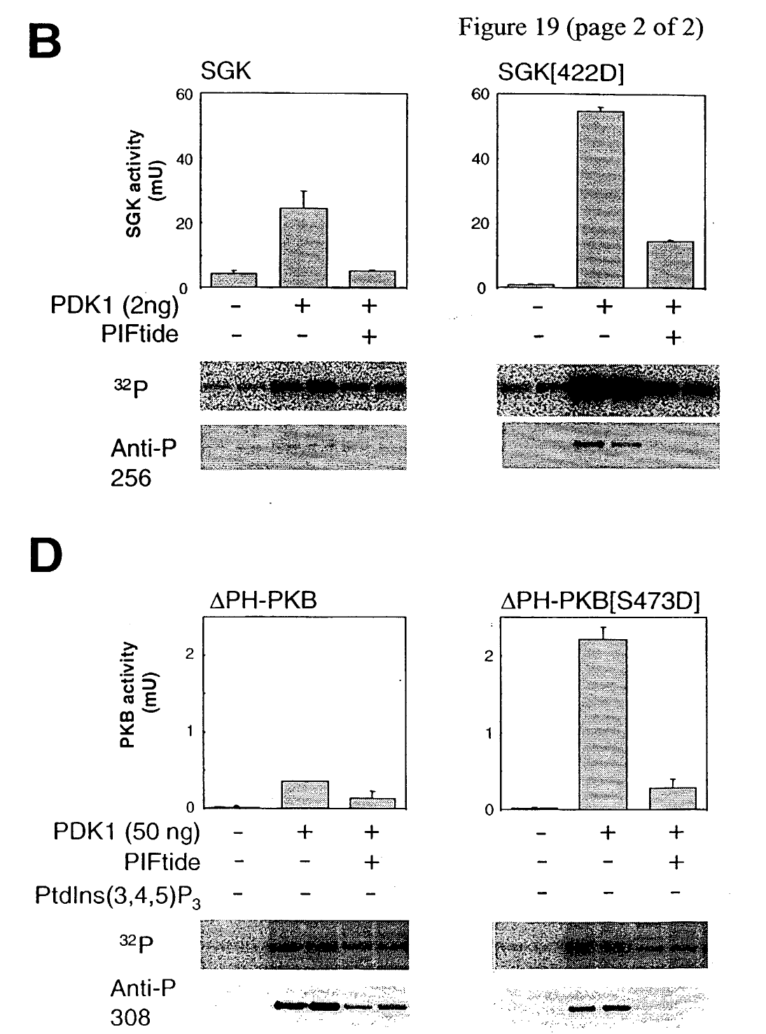

FIG. 19. Effect of PIFtide on the in vitro phosphorylation and activation of PDK1 substrates. Substrates (0.6 μM) were incubated in vitro with GST-PDK1 as indicated in the presence or absence of PIFtide (2).

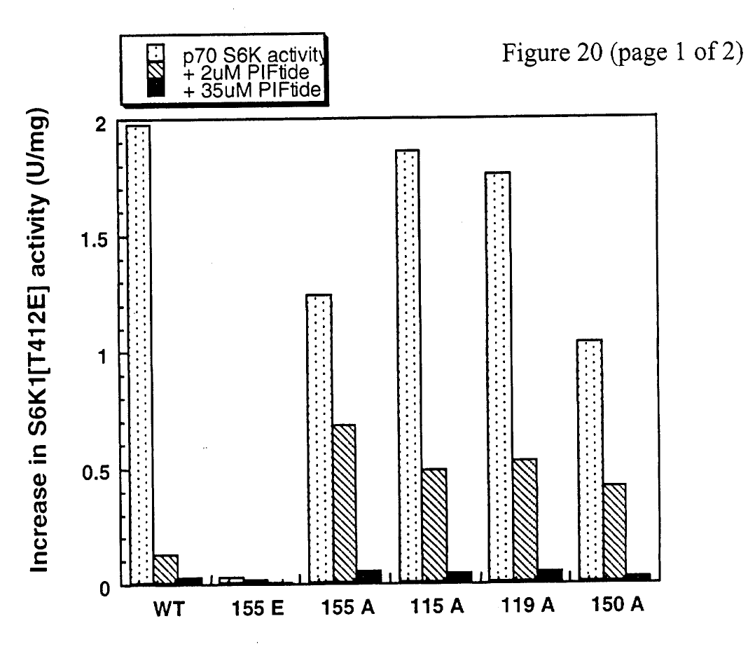

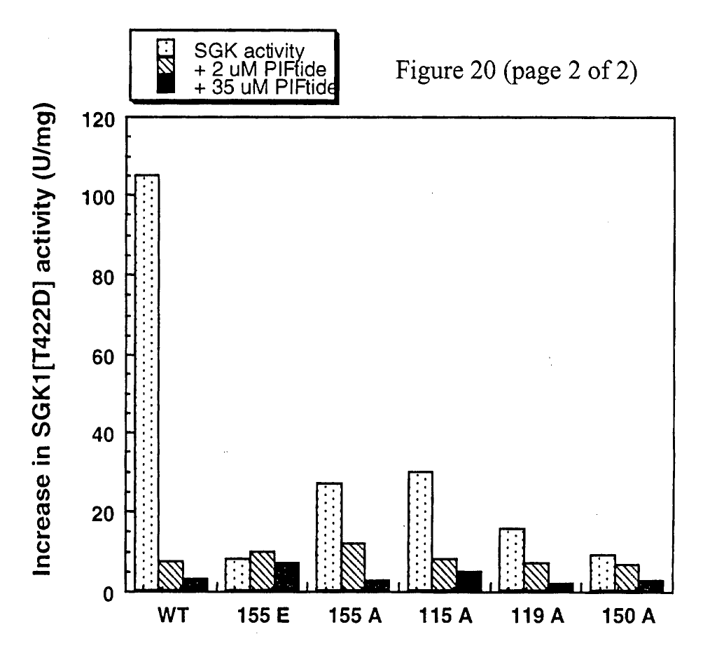

FIG. 20. Effect of PIFtide on the activation of S6K1 and SGK1 by PDK1 PIF pocket mutants (155A, 115A, 119A, 150A). GST-PDK1, GST-PDK1 L155E, 155A, 115A, 119A and 150A were tested for their ability to activate His-S6K1 412E and GST-SGK1 422D. The phosphorylation and activation of substrates was performed as described in FIG. 17. When PIFtide was included in the reaction, it was pre-incubated on ice for ^{˜}15 min until the reaction was initiated with the addition of ATP-Mg.

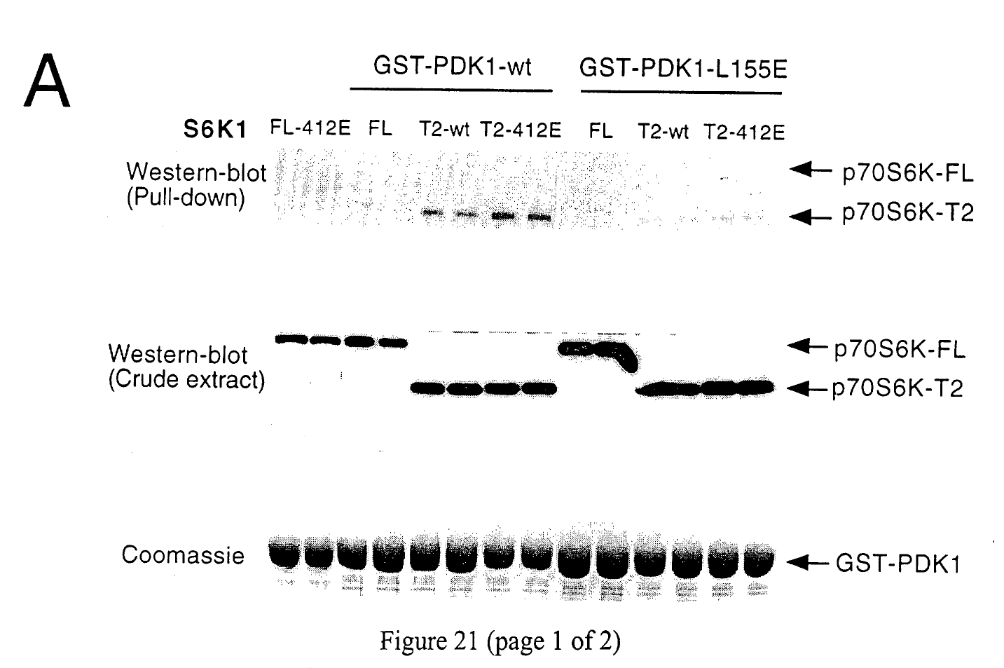

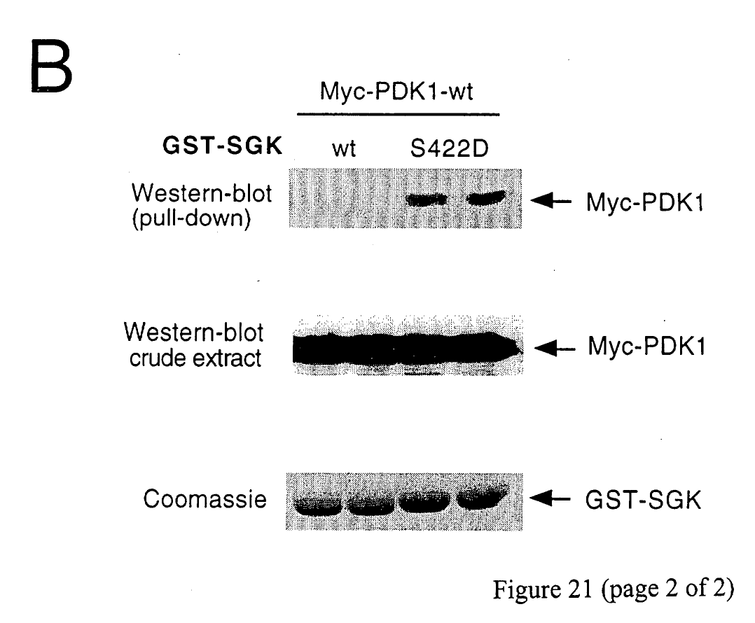

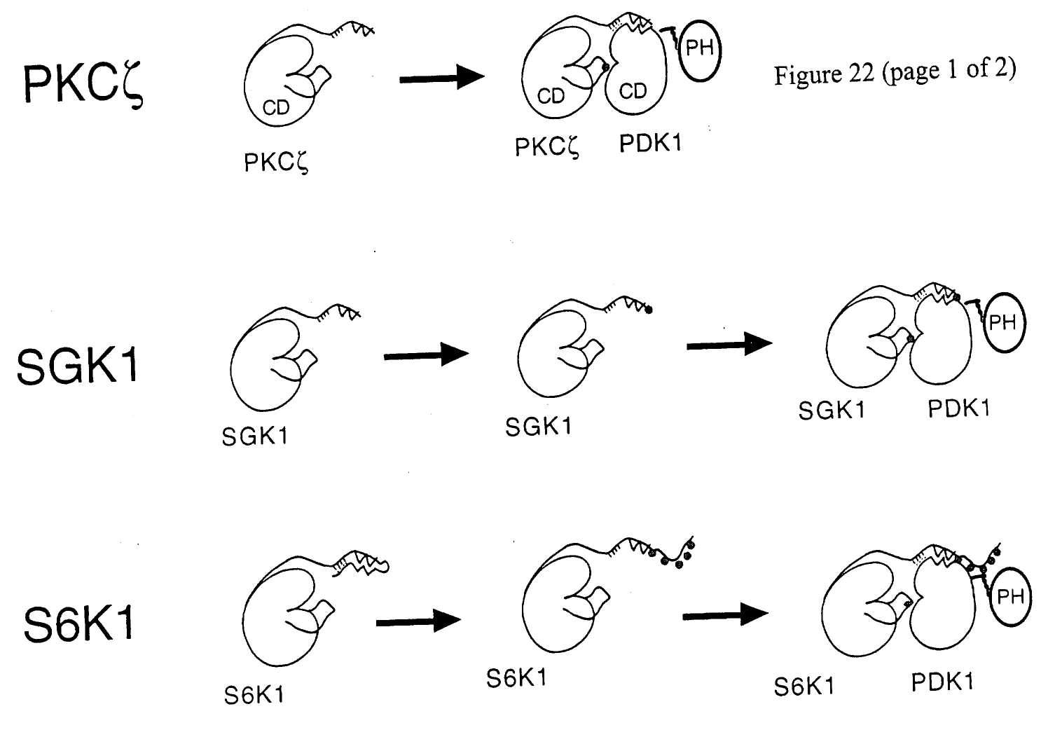

FIG. 21. Interaction of S6K1 and SGK1 with PDK1. 293 cells were transiently transfected with DNA constructs expressing GST, GST-PDK1 wt or PDK1 L155E together with constructs expressing either wild type or the indicated mutants or truncations of HA tagged S6K1 (A) or wild type GST-ΔN-SGK1 or 422D mutant. 36 h post transfection the cells were lysed and GST fusion protein was purified by affinity chromatography on glutathione-Sepharose beads. Aliquots were electrophoresed on a 10% SDS-polyacrylamide gel, stained with Coomassie Blue, or immunoblotted using an anti-FLAG antibody to detect FLAG-S6K1 and anti-Myc antibody to detect Myc-PDK1. To establish that the wild type and mutant proteins were expressed at similar levels, 10 mg of total lysate was electrophoresed on a 10% SDS-polyacrylamide gel and immunoblotted using the indicated antibodies. Duplicates of each condition are shown. Similar results were obtained in two different experiments.

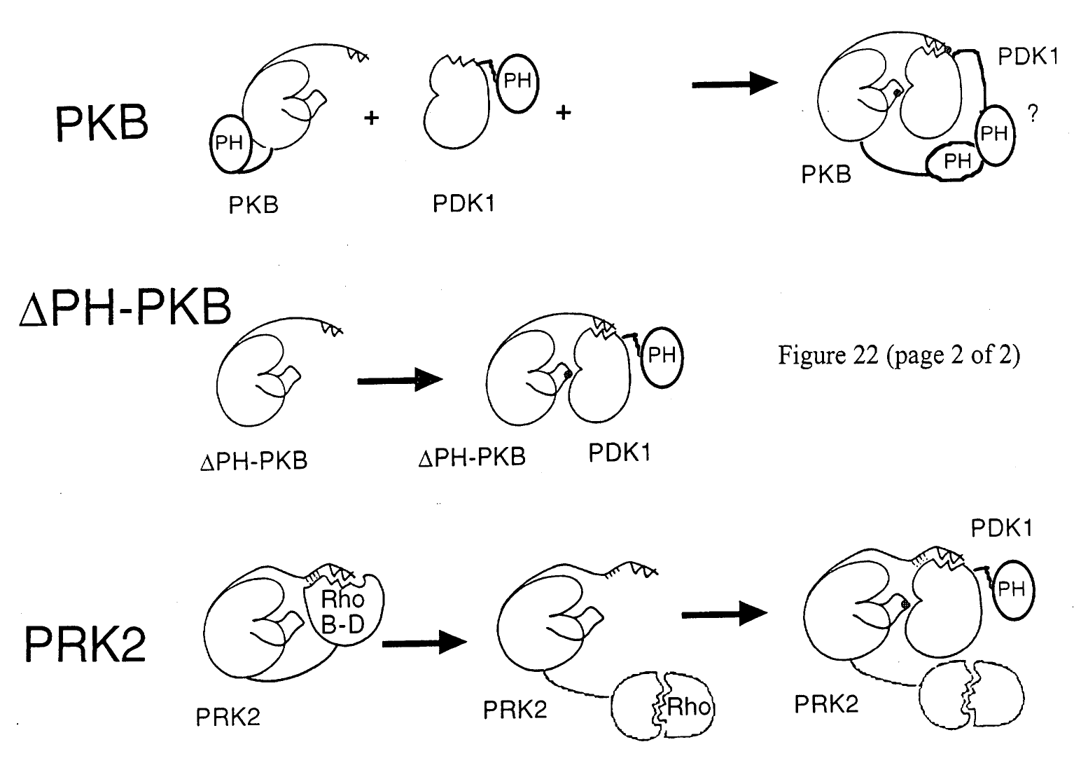

FIG. 22. Model for PDK1 specificity. Over-expression of substrates of PDK1 in 293 cells produces protein kinases which are constitutively phosphorylated (PKCζ and PRK2), others that are phosphorylated and activated within 1-2 minutes of IGF1 stimulation (PKB), and others that are phosphorylated and activated after 10-40 minutes of exposure to the same stimulus (S6K and SGK). Thus, there should be a mechanism that ensures this particular specificity. Here we depict a possible model for the phosphorylation of PDK1 substrates that is supported by the results here presented and is in agreement with published observations. In this model, PDK1 activity needs not be regulated. Rather, modifications on substrates other than PKB could allow the direct interaction of the C-terminal hydrophobic motif of these kinases with the PIF binding pocket of PDK1. Interaction with its substrates by this means would be the regulatory step ensuring the temporal and spacial specificity of PDK1. After synthesis, PKCζ would be in a conformation that enables its direct interaction with PDK1 and hence it is constitutively phosphorylated □. In 293 cells the overexpression of PRK2 leads to a similarly active (phosphorylated) enzyme, but it is suggested that the interaction of PRK2 with PDK1 could be regulated by Rho □. SGK and S6K must be modified by phosphorylation in order to allow the interaction with PDK1, which prompts their phosphorylation and activation. The main interaction between PKB and PDK1 is likely to be dependent on PtdIns(3,4,5)P3 possibly by lipid mediated co-localisation. In PKB interaction, a minor role could be played by PDK1 PIF pocket, since ΔPH-PKB phosphorylation is dependent on the hydrophobic motif—PIF binding pocket interaction.

### EXAMPLE 1

### Evidence that PDK1 Phosphorylates p70 S6 Kinase in vivo at Thr412 as Well as Ser252.

Abbreviations: PKB, Protein kinase B;PtdIns, Phosphatidylinositol;PI3-kinase, Phosphoinositide 3-kinase; PH, pleckstrin homology; RSK, Ribosomal S6 kinase; MSK, Mitogen and Stress Stimulated kinase; 1,2-SAD-PtdIns(3 ,4,5)P₃,sn-1-stearoyl-2-arachidonoyl-D-PtdIns(3,4,5)P₃;C₁₆-PtdIns(3,4,5)P₃,sn-1,2 di-palmitoyl-D-PtdIns(3,4,5)P₃;C₁₆-PtdIns(3,4)P₂,sn-1,2 di-palmitoyl-D-PtdIns(3,4)P₂.

In this Example, we demonstrate that PDK1 expressed in cells, for example 293 cells or bacteria, is capable of phosphorylating p70 S6 kinase at Thr412 in vitro. We find that PDK1 bound to PIF is no longer able to interact with or phosphorylate p70 S6 kinase in vitro at either Thr252 or Thr412. The expression of PIF in cells prevents IGF1 from inducing the activation of the p70 S6 kinase and its phosphorylation at Thr412. Overexpression of PDK1 in cells induces the phosphorylation of p70 S6 kinase at Thr412 in unstimulated cells, and a catalytically inactive mutant of PDK1 prevents the phosphorylation of p70 S6K at Thr412 in IGF1-stimulated cells. These observations provide further evidence that PDK1 is one of the kinases that regulates the activation of p70 S6 kinase, and the first evidence that PDK1 mediates the phosphorylation of p70 S6 kinase at Thr412 in cells.

Experimental Procedures

Materials The peptides used to assay PKBα, (RPRAATF) [23] p70 S6K (GRPRTSSFAEG) [24] and the peptides used to raise and purify the T412-P antibody were synthesised by Dr G. Blomberg (University of Bristol, U.K). Protein G-Sepharose, glutathione Sepharose and CHX-Sepharose were purchased from Pharmacia; Protease-cocktail tablets from Roche, tissue culture reagents, IGFI and microcystin-LR were from Life Technologies; sensorChips CM5 and SA were from BiaCore AB; biotinylated reagent and secondary antibodies coupled to horse radish peroxidase were from Pierce.

Antibodies. The phospho-specific antibody recognising p70 S6K phosphorylated at Thr412 was raised in sheep against the peptide SESANQVFLGFTYVAPSV (corresponding to residues 401 to 418 of the longer splice variant of human α-isoform of p70 S6K), in which the underlined residue is phosphothreonine. The antibody was affnity purified on CH-Sepharose covalently coupled to the phosphorylated peptide. The antibodies were then passed through a column coupled to the non-phosphorylated peptide and the antibodies that did not bind to this column were selected. Monoclonal antibodies recognising the HA or Myc epitope were purchased from Boehringer Mannheim, the monoclonal antibody recognising GST was purchased from Sigma and used to verify the level of expression of GST-PIF in cells, white rabbit polyclonal antibodies recognising the 18 C-terminal residues of PRK2/PIF were purchased from SantaCruz Biotechnology.

Preparation of insect cell His-p70 S6K. p70 S6K with a His-epitope tag at its N-terminus lacking the carboxy terminal 104 residues is termed p70 S6K-T2. In order to prepare wild type and the mutant 412E-p70 S6K-T2 the cDNA for these constructs was amplified by PCR from the pMT2 vector encoding these forms of p70 S6K [6] using the following oligonucleotides:5′-AGG ATC CAC CAT GCA CCA TCA CCA TCA CCA TAT GAG GCG AGC AAG GAG GCG G-3′ and 5′-GCG GCC GCT CAA CTT TCA AGT ACA GAT GGA GCC-3′. The PCR products were then subcloned into the BamH1/Not1 sites of the pFASTBAC 1 vector and this vector was used to generate recombinant baculovirus using the Bac-to-Bac system (Life Technologies Inc). The resulting viruses, encoded p70 S6K-T2 or 412E-p70 S6K-T2 with an N-terminal hexahistidine sequence, and was used to infect Sf21 cells (1.5×10⁶/m) at a multiplicity of infection of 5. The infected cells were harvested 72 h post-infection and the His-p70 S6K proteins purified by Ni²⁺/NTA (nitrilotriacetic acid)-agarose chromatography as described previously for PKBβ [25]. They were then dialysed against 50 mM Tris/HCl pH 7.5, 0.1 mM EGTA, 0.27 M sucrose, 0.03% (by vol) Brij-35, 0.15 (by vol) 2-mercaptoethanol, 1 mM benzamidine and 0.2 mM phenylmethylsuphonyl fluoride, snap frozen in aliquots and stored at −80° C. p70 S6K-T2 or 412E p70 S6K-T2 were both recovered with a yield of 60 mg/litre of infected Sf21 cells and were >90% homogeneous as judged by polyacrylamide gel electrophoresis followed by Coomassie Blue staining.

Phosphorylation of GST-p70 S6K by PDK1. GST-PIF and GST-D978A-PIF were expressed in human embryonic kidney 293 cells, purified on glutathione-Sepharose, and the very small amount of endogenous PDK1 associated with GST-PIF was removed by immunoprecipitation with a PDK1 antibody [22]. Phosphorylation of GST-p70 S6K-T2 by PDK1 was carried out as described previously [7] except that PDK1 was incubated with the indicated concentration of GST-PIF or PIF peptide for 10 min on ice prior to initiation of the assay with Mg[γ³²PATP]. The wild type GST-p70 S6K-T2, and the mutant T252A-GST-p70 S6K-T2, T412A GST-p70 S6K-T2 proteins were expressed in 293 cells and purified as described previously [7]. Wild type and catalytically inactive GST-PDK1 was expressed either in 293 cells [26] or in E.coli [27]

Transient Transfection Experiments. The DNA constructs encoding for the wild type and mutant forms of HA-p70 S6K in the pMT2 vector used in this study have been described previously [6]. The constructs encoding wild type HA-PKBα [25]; wild type Myc-PDK [26] and a catalytically inactive mutant of Myc-PDK1, (in which Lys 111 and Asp223 are both mutated to Ala) termed kinase-dead PDK1 [26], were all in the pCMV5 vector. The constructs used to express GST-PIF and the mutant GST-F977A-PIF [22] are in the pEBG2T vector. The empty pEBG2T vector was used to express GST protein in control experiments. DNA constructs used in this study were purified from bacteria using the Qiagen plasmid Mega kit according to the manufacturer's protocol.

293 cells cultured on 10 cm diameter dishes in Dulbecco's Modified Eagle's Medium containing 10% (by vol) foetal bovine serum, were transfected with 2 μg of DNA construct encoding either wild type or mutant HA-p70 S6K or HA-PKBα, and 10 μg of DNA construct encoding either GST-PIF, GST-F977A-PIF, GST, Myc-PDK1, kinase-dead Myc-PDK1, or the empty pCMV5 vector using a modified calcium phosphate method [28]. 24 h post transfection the cells were deprived of serum for 16 h, and exposed to IGF1 (100 nM) for the time indicated. The cells were lysed in 1 ml of lysis buffer (50 mM Tris/HCl pH 7.5, 1 mM EDTA, 1 mM EGTA, 1% (by vol) Triton X-100, 1 mM sodium orthovanadate, 10 mM sodium β-glycerophosphate, 50 mM NaF, 5 mM sodium pyrophosphate, 1 μM microcystin-LR, 0.27 M sucrose and protease cocktail tablets), cleared by centrifugation, and 50 μg of protein was subjected to immunoprecipitation with anti HA monoclonal antibody. The protein concentrations of the lysates were determined by the Bradford method.

Kinase assays. The HA-p70 S6K or HA-PKBα immunoprecipitates were washed and assayed for kinase activity using the peptide Crosstide (GRPRTSSFAEG) as described previously for PKBα [28]. One unit of activity, U, was that amount which catalysed the phosphorylation of 1 nmol of substrate in one minute.

Immunoblotting for dephosphorylated and Thr412-phosphorylated p70 S6 kinase. Cell lysates were made 1% SDS and the indicated amounts of protein were subjected to SDS/Polyacrylamide gel electrophoresis, subsequently transferred to nitrocellulose and immunoblotted using the indicated monoclonal antibody or the T412-P phospho-specific antibody (0.4 μg/ml) in 50 mM Tris/HCl pH7.5, 0.15M NaCl, 0.5% (by vol) Tween and 10% (by mass) skimmed milk. Detection was performed using the enhanced chemiluminescence reagent (Amersham Pharmacia Biotech).

Surface plasmon resonance measurements of PDK1 binding to p70 S6 kinase.

p70 S6K-T2 and T412Ep70S6K-T2 mutant were amine coupled to a CM5 sensor chip (BIAcore AB) according to the manufacturer's instructions. The indicated concentrations of His-PDK1 was injected over the chip at a flow rate of 30 μl/min and the steady-state binding determined, in the presence or absence of PIF peptide. The apparent equilibrium dissociation constant (K_{d}) for the binding of His-PDK1 to p70 S6 kinase was determined by fitting the increase in steady-state binding upon increased PDK1 concentration to a rectangular hyperbola using SigmaPlot 4 (SPSS Inc). The measure of response in our experiments is termed RU; 1000 RU=1 ng/mm² of protein bound to the surface.

Results

Phosphorylation of p70 S6K by PDK1 is inhibited by PIF. PDK1 binds with submicromolar affinity to a region of Protein Kinase C-Related Kinase-2 (PRK2), termed the PDK1-Interacting Fragment (PIF) [22]. PIF is situated C-terminal to the kinase domain of PRK2, and the binding of this region of PRK2 to PDK1 is mediated by a consensus motif similar to that encompassing Thr412 of p70 S6K, except that the residue at this position is Asp (Asp978), rather than Thr or Ser. In FIG. 1, we demonstrate that PDK1 when complexed to either GST-PIF or a 24 residue synthetic peptide whose sequence encompasses the PDK1 binding site on PIF (PIFtide), was unable to phosphorylate GST-p70 S6K-T2 (a deletion mutant of p70 S6K which lacks the C-terminal 104 residues) in vitro. In a parallel experiment it was verified that PDK1 complexed to GST-PIF or the PIF peptide, was able to phosphorylate PKB at both Thr308 and Ser473 to near stoichiometric levels (data not shown) as reported previously [22]. GST-p70 S6K-T2 was used as a PDK1 substrate (FIG. 1) rather than the full length p70 S6K which is very poorly phosphorylated by PDK1 in vitro [7,8]. Truncation of the C-terminal 104 residues of p70 S6K is likely to be benign, as p70S6K-T2 when expressed in cells possesses indistinguishable properties to the full length protein as it is still activated by insulin and growth factors in a rapamycin and wortmannin sensitive manner [5+6].

A mutant form of GST-PIF or the 24 residue PIF peptide in which the amino acid equivalent to Asp978 in PRK2 is mutated to Ala (GST-D978A-PIF), possesses markedly reduced affinity for PDK1 [22]. Consistent with this, GST-D978A-PIF or the mutant D978A-PIF peptide poorly inhibited the phosphorylation of GST-p70 S6K-T2 by PDK1 slightly (FIG. 1). If Asp978 in the PIF peptide is mutated to a phosphoserine residue instead of an Ala, to restore the negative charge, the resulting peptide interacted with PDK1 with the same affinity as the wild type PIF peptide [22] and prevented PDK1 from phosphorylating GST-p70 S6K-T2 (FIG. 1).

PDK1 phosphorylates p70 S6 kinase at Thr412 in vitro. In order to determine whether PDK1 could phosphorylate p70 S6K at Thr412, we raised phospho-specific antibodies that only recognise p70 S6K phosphorylated at Thr412 (termed T412-P antibody). This antibody did not recognise GST-p70 S6K-T2 that had been incubated with MgATP in the absence of PDK1. However, following the addition of PDK1 which had either been expressed in 293 cells or bacteria, the GST-p70 S6K-T2 became recognised by the T412-P antibody (FIG. 2). Incubation of the T412-P antibody with the phosphorylated Thr412 peptide immunogen used to raise the antibody (but not with the dephosphorylated peptide) abolished its recognition of GST-p70 S6K-T2 (see FIG. 3). The rate at which PDK1 phosphorylated T412 (as well as Thr252) of GST-p-70 S6K-T2 was not increased in the presence of lipid vesicles containing phosphatidylinositol 3,4,5-trisphosphate (data not shown). PDK1 phosphorylated the T252A mutant of GST-p-70 S6K-T2 at Thr412 to the same extent as the wild type GST-p70 S6K-T2. The T412A mutant of GST-p70 S6K-T2 was not recognised by the T412-P antibody after incubation with PDK1/MgATP. The 24 residue PIF peptide prevented PDK1 from phosphorylating the p70 S6K at Thr412. A kinase-dead mutant of PDK1 was unable to phosphorylate GST-p-70 S6K-T2 at Thr412 (FIG. 2).

The rate at which PDK1 phosphorylates Thr412 is likely to be significantly lower than that at which it phosphorylates Thr252. ³²P-labelled GST-p70 S6K-T2 phosphorylated with PDK1 was digested with either trypsin or V8 protease and then subjected to peptide map analysis on HPLC as described previously [7]. This analysis revealed that the major ³²P-labelled peptide containing 20-30% of the total radioactivity applied to the HPLC column corresponding to the peptide phosphorylated at Thr252. Although several minor peptides were present in this analysis which each comprised <5% of the total phosphate, we were unable to attribute any of these to a peptide phosphorylated at Thr412. This analysis does not exclude the possibility that the recovery of the ³²P-labelled peptide phosphorylated at Thr412 may be poor but suggests that the stoichiometry at which PDK1 phosphorylated p70 S6K at Thr412 is much lower than that which it phosphorylates Thr252.

PIF inhibits IGF1-induced activation of p70 S6K. In order to determine whether expression of PIF in cells could prevent the endogenous PDK1 from phosphorylating and activating p70 S6K, HA-tagged full length p70 S6K (HA-p70 S6K) was transfected into 293 cells together with constructs encoding either GST-PIF, a mutant form of GST-PIF which interacts with PDK1 weakly (GST-F977A-PIF) or GST itself. The wild type or mutant GST-PIF and GST itself were all expressed at a similar level, and were present at a much higher concentration than the endogenous PDK1 or PRK2 (data not shown). The cells were subsequently stimulated with IGF1 for 40 min (the time at which HA-p70 S6K is maximally activated, data not shown), the cells lysed and the HA-p70S6K immunoprecipitated and assayed. Cells expressing HA-p70 S6K and GST exhibited a readily measurable basal p70 S6K activity in unstimulated cells, which was increased 10-fold in response to IGF1 (FIG. 3A). In contrast, cells expressing HA-p70 S6K and GST-PIF, possessed a basal HA-p70 S6K activity that was virtually undetectable, and IGF-stimulation caused only a very slight increase in the HA p70 S6K activity (FIG. 3A). In cells expressing HA-p70 S6K and GST-F977A-PIF, HA-p70 S6K was substantially activated by IGF1, although not to the same extent as in cells expressing HA-p70 S6K and GST (FIG. 3A). This is probably explained by a weak interaction of GST-F977A-PIF with PDK1.

PIF inhibits IGF1 induced phosphorylation of p70 S6K at Thr412. As PIF inhibited P70 S6K activation in cells, we sought to determine the effect of PIF expression on the phosphorylation of p70 S6K at Thr412 and Thr252. We used the T412-P antibody to measure the phosphorylation of p70 S6K at Thr412. These experiments showed that IGF1 triggered the phosphorylation of Thr412 (FIG. 3A). This was abolished by incubation of the T412-P antibody with the phosphorylated Thr412 peptide immunogen used to raise the antibody (but not with the dephosphorylated peptide (FIG. 3A) or a phosphopeptide corresponding to the sequence surrounding Thr252 (data not shown). Furthermore, a mutant form of HA-p70 S6K in which Thr412 was changed to an Ala was not recognised by the T412-P antibody following IGF1 simulation (FIG. 5C).

When HA-p70 S6K was coexpressed in cells with GST-PIF, IGF1 failed to induce the phosphorylation of HA-p70 S6K at Thr412 (FIG. 3A). In contrast in cells expressing HA-p70 S6K and the mutant GST-F977A-PIF, the phosphorylation of HA-p70 S6K still occurred but a lower level than that observed in cells expressing HA-p70 S6K and GST. The decrease in Thr412 phosphorylation is consistent with the reduced activation of HA-p70 S6K in these cells compared to those expressing GST alone (FIG. 3A). It should be noted however that contransfection of HAp70 S6K with the GST-F977A-PIF mutant induced a 50% maximal activation of P70 S6K, despite inducing a significantly greater reduction in the level of phosphorylation of T412 (FIG. 3A). This finding demonstrates that the relationship between p70 phosphorylation at Thr412 and level of p70 S6K activity does not appear to be linear. One explanation for this observation is that the F977A-PIF mutant may inhibit more potently p70 S6K phosphorylation at Thr412 than Thr252; however, thus far we have not been able to raise phosphospecific antibodies recognising p70 S6K phosphorylated at Thr252 to explore this possibility.

The overexpression of GST-PIF in cells also abolished the IGF1 induced activation and phosphorlation at Thr412 of the p70 S6K-T2 mutant which lacks the C-terminal 104 residues (data not shown).

PIF inhibits IGF1-induced phosphorylation of p70 S6K at Thr252. A mutant form of HA-p70 S6K in which Thr412 was altered to glutamic acid to mimic the presence of a phosphorylated residue at this position, possessed an elevated basal activity which was further activated by IGF1 when co-expressed with GST or the mutant GST-F977A-PIF (FIG. 3B). Previous work has established that the basal and IGF1-stimulated activity of this mutant, is mediated through phosphorylation of Thr252 [6]. In FIG. 3B, we demonstrate that co-expression of HA-412E p70 S6K with PIF greatly reduced the basal activity of this mutant and largely prevented its activation by IGF1. This suggests that PIF also inhibits the phosphorylation of p70 S6K at Thr252. The overexpression of PIF cells also greatly reduced the basal and IGF1-stimulated activity of T412E p70 S6K-T2 mutant in cells (data not shown).

PIF does not inhibit the activation of PKBα or its phosphorylation at Ser473. Previous work has shown that PIF does not prevent PDK1 from phosphorylating PKB in the presence of 3-phosphoinositide lipids but instead enables PDK1 to phosphorylate PKB at both Thr308 and Ser473 (see introduction). Here we show that in marked contrast to the effect of GST-PIF on p70 S5K activation, expression of GST-PIF in 293 cells does not prevent IGF1 from inducing a ˜20-fold activation of HA-PKBα. This activation is similar to that observed when HA-PKBα is coexpressed with GST (FIG. 4). Expression of GST-PIF did not inhibit or potentiate the IGF1-induced phosphorylation of HA-PKBα at Ser473 (the residue equivalent to Thr412 in p70 S6K) (FIG. 4). GST-PIF is expressed at a similar level when contransfected with PKB and HA-p70 S6K (data not shown), indicating that the inability of PIF to affect the activation of PKB in cells is not due to it being expressed at a low level.

A catalytically inactive mutant of PDK1 prevents the activation and phosphorylation of p70 S6K.

Consistent with earlier findings [7,8], co-expression of HA-p70 S6K with wild type PDK1 induced a large activation of HA p70 S6K which was not increased further by IGF1-stimulation (FIG. 5A). We consistently observed a slight decrease in HA-p70 S6K activity in cells overexpressing PDK1 following IGF-stimulation. The co-expression of wild type PDK1 with HA-p70 S6K or T252A-p70 S6K also resulted in a large increase in Thr412 phosphorylation in unstimulated cells (FIGS. 5A & 5B). In contrast, no immunoreative band was detected after immunoblotting with the T412-P antibody, when wild type PDK1 and the HA-T412A p70 S6K mutant were co-expressed (FIG. 5C). When a kinase-dead mutant of PDK1 was co-expressed with HA-p70 S6K, the latter was no longer activated following IGF1-stimulation of cells, nor was it phosphorylated at Thr412 (FIG. 5A). In FIG. 5D we demonstrate that co-expression of HA-412E p70 S6K with a catalytically inactive PDK1 reduced the basal level of HA-412E p70 S6K and largely prevented its activation by IGF1. This provides evidence that the overexpression of a kinase dead PDK1 in cells also inhibits the phosphorylation of p70 S6K at Thr252.

PIF prevents the interaction of PDK1 with p70 S6 kinase. A recent study by Blenis and colleagues [29] reported that, when PDK1 and p70 S6K were contransfected into cells, a small amount of PDK1 was coimmunoprecipitated with p70 S6K, suggesting that these proteins may interact directly. Using Surface Plasmon Resonance measurements, we were able to detect a significant interaction (apparent K_{d} 8 μM) between PDK1 and p412E-p70 S6K-T2 (FIG. 6). This interaction was abolished in the presence of the 24 residue wild type PIF peptide but not the Asp978Ala mutant of the PIF peptide (FIG. 6), suggesting that both PIF and 412E-p70 S6K-T2 mutant compete for the same binding site on PDK1. In parallel experiments, a significantly weaker interaction between PDK1 and wild type p70 S6K-T2 kinase was detected (FIG. 6).

Discussion

Recent work has shown a high affinity-interaction between PIF and the kinase domain of PDK1, which enhances the rate at which PDK1 phosphorylates PKBα and allows it to phosphorylate Ser473 as well as Thr308. In this study we have made the surprising observation that PIF prevents PDK1 from phosphorylating p70 S6K (FIGS. 1 and 2) and expression of PIF in 293 cells prevents the IGF1-induced activation of p70 S6K (FIG. 3) without affecting the activation of PKBα (FIG. 4). Mutant forms of PIF which interact weakly with PDK1 were much less effective at inhibiting the phosphorylation of p70 S6K by PDK1 in vitro, or at inhibiting the IGF1-induced activation of the p70 S6K. These observations could be explained if p70 S6K, but not PKBα, needed to interact with PDK1 at a site which overlaps with the PIF binding site, before p70 S6K can become phosphorylated by PDK1. This conclusion is supported by the findings in FIG. 6 that p70 S6K does interact with PDK1, and this interaction is abolished in the presence of the PIF Peptide. The finding that the T412E mutant form of p70 S6K interacts with PDK1 with higher affinity than the wild type enzyme, may also explain why the T412E mutant of p70 S6K was observed in previous studies to be a better substrate for PDK1 than the wild type or T412A mutant of p70 S6K [7,8]. Phosphorylation of PKBα by PDK1 is not inhibited by the presence of PIF, and nor could we detect any significant interaction between PKBα and PDK1 in vitro by surface plasmon resonance (data not shown). As PKBα and PDK1 both interact with 3-phosphoinositides through their PH domains, it is possible that this is the primary determinant for co-localising these molecules at the plasma membrane and hence allowing PDK1 to phosphorylate PKBα. In contrast, substrates for PDK1 such as p70 S6K, which do not interact with 3-phosphoinositides may actually need to interact with PDK1 with relatively high affinity, before they can become phosphorylated. Previous evidence that PDK1 is an activator of p70 S6K rested largely on the demonstration that PDK1 phosphorylates and activates p70 S6K in vitro and in cotransfection experiments. The finding in this study that expression of PIF can prevent the activation of p70 S6K in vivo, presumably by binding to PDK1, provides further evidence that PDK1 is required for the activation of p70 S6K in cells.

Interaction with PIF converts PDK1 into a kinase that is capable of phosphorylating both Thr308 and Ser473 sites of PKB. This demonstrates that PDK1 has the intrinsic ability to phosphorylate the residue in the T-loop as well as the PDK2 motif of a least one AGC kinase family member. As the residues surrounding Thr412 of p70 S6K are highly homologous to those surrounding Ser473 of PKB, it was possible that PDK1, probably in complex with another protein(s), would also possess the intrinsic ability to phosphorylate p70 S6K at Thr252 and Thr412. The present study supports this hypothesis because firstly, overexpression of wild type PDK1 triggers the phosphorylation of p70 S6K at Thr412 (FIG. 5A). Secondly, the PDK1-catalysed phosphorylation of p70 S6K at Thr412 in vitro was prevented by PIF (FIG. 2). Thirdly, expression with PIF prevents the IGF-induced phosphorylation of p70 S6K at Thr412 in cells (FIG. 3A). Finally, the overexpression of a kinase-dead mutant of PDK1 in cells not only prevented the activation of p70 S6, as reported by others [8], but also prevented the phosphorylation of p70 S6K at Thr412 (FIG. 5). Taken together, the data suggests that PDK1 could phosphorylate p70 S6K at Thr412 in vivo. As PDK1 phosphorylation of Thr412 of p70 S6K in vitro is not dependent upon 3-phosphoinositde lipids, it is possible that the sensitivity of PDK1 to these lipids in cells is conferred by the interaction of PDK1 with another protein. In this respect it should be recalled that the interaction of PDK1 with PIF enables PDK1 to be directly activated by 3-phosphoinositides [22]. It is also possible that a PDK1-interacting protein (s) could increase the rate at which PDK1 phosphorylates both Thr252 and Thr412 of p70 S6K.

It has been recently reported that catalytically inactive mutants of PKCλ[30] and PKCξ[29] antagonise the ability of agonists to activate p70 S6K in cells. These observations were interpreted as indicating that PKCλ/PKCξ may have a role in activating p70 S6K in cells. However, PKCλ and PKCζ are both AGC kinase family members which are likely to be activated by PDK1 in vivo, and possess an acidic residue rather than Ser/Thr in their PKD2 consensus motif. Furthermore, PKCζ, like PIF has been shown to interact directly with the kinase domain of PDK1 [16,18]. It is therefore possible that both PKCλ and PKCζ interact with PDK1 in the same way as PIF, and so prevent PDK1 from inducing the activation of p70 S6K. Recent work also implicated PKCζ in mediating a rapamycin-sensitive phosphorylation of the novel PKC isoform (PKCδ) at the residue equivalent to Thr412 of p70 S6K [31]. This study did not, however rule out the possibility that PDK1 complexed to PKCζ acquires the ability to phosphorylate PKCδ at this residue, rather than PKCζ itself directly phosphorylating this residue. To complicate matters further, it has also recently been shown that conventional PKCα is capable of autophosphorylating itself at the residue equivalent to Thr412 of p70 S6K [32, reviewed 33]. Sabatini and colleagues have reported that mTOR phosphorylates p70 S6K directly at Thr412 [34]. However, much recent evidence suggests that the ability of rapamycin, an inhibitor of the mTor kinase, to suppress the activity of p70 S6K is mediated primarily through the rapamycin-induced activation of an mTor-regulated protein phosphatase which dephosphorylates p70 S6K [35-37]. It will be important to establish whether mTor or any other insulin-stimulated kinase, which can phosphorylate p70 S6K at Thr412 is inhibited by PIF.

References

1. Proud, C G. (1995). Trends in Bioch. Sci 21, 181-185.

1. Lane et al (1993). Nature 363,170-172.

2. Jefferies et al (1997) EMBO J. 16, 3693-3704.

3. Pearson et al (1995) EMBO J. 14, 5278-5287.

4. Pullen N & Thomas G. (1998) FEBS LETT.410, 78-82.

5. Weng et al (1998) J. Biol. Chem.273, 16621-16629.

6. Alessi et al (1998) Curr. Biol. 8, 69-81.

7. Pullen et al (1998). Science, 279:707-710.

8. Alessi D R. & Cohen P. (1998). Curr. Opin. Genetics. Develop. 8:55-62.

9. Mellor H & Parker P. J. (1998) Biochem. J., 332:281-292.

10. Webster et al (1993). Mol. Cell.Biol.13, 2031-2040.

11. Shepherd et al (1998). Biochem. J. 333: 471-479.

12. Belham et al (1999). Current Biol. 9, R93-R96.

13. Alessi et al (1997). Curr. Biol. 7:261-269.

14. Stokoe et al (1997). Science 277:567-570.

15. LeGood et al (1998). Science 281:2042-2045.

16. Chou et al (1998). Curr. Biol., 8: 1069-1077.

17. Dutil et al (1998). Curr.Biol.8:1366-1375.

18. Kobayashi T & Cohen P. (1999) Biochem. J. 339, 319-328.

19. Park et al (1999). EMBO J.18, 3024-3033.

20. Cheng et al (1998). Proc. Natl. Acad. Sci. USA 95:9849-9854.

21. Balendran et al (1999). Current Biology 9, 393-404.

22. Alessi et al (1996). FEBS Lett 399, 333-338.

23. Leighton et al (1995). FEBS Lett. 375, 289-293.

24. Walker et al (1998). Biochem. J. 331, 299-308.

25. Alessi et al (1997). Curr.Biol.7, 776-789.

26. Casamayor et al (1999). Biochem. J. 342, 287-292.

27. Alessi et al (1996). EMBO J.15:6541-6551.

28. Romanelli et al (1999) J. Mol. Cell. Biol 19, 2921-2928.

29. Akimoto et al (1998) Biochem. J. 273, 16621-16629.

30. Ziegler et al (1999). Curr. Biol. 9, 522-529.

31. Dutil et al (1999) Curr. Biol. 8, 1366-1375.

32. Peterson et al (1999) Current Biol. 9, R521-524.

33. Burnett et al (1998). Proc.Natl. Acad. Sci. 95, 1432-1437.

34. Hara et al (1998) J. Biol. Chem 273, 22160-22168.

35. Peterson et al (1999) Proc. Natl. Acad. Sci. 96, 4438-4442.

37.Sabatini et al (1999). Science 284, 1161-1164.

### EXAMPLE 2

### Identification of a Hydrophobic Pocket in the Small Lobe of the PDK1 Kinase Domain Which Interacts with PIF and the C-Terminal Residues of PKA

Abbreviations used (other than those defined in Example 1): PKA, cAMP dependent protein kinase; PKA_{CT}, C-terminal fragment of PKA composed of residues 129-350; PH, pleckstrin homology; PIF; PDK1 interacting fragment.

Materials and Methods

Materials

Complete protease inhibitor cocktail tablets and anti-Myc monoclonal antibodies were from Roche; tissue culture reagents were from Life Technologies: SensorChips SA were from BiaCore AB; biotinylated reagent and secondary anti-mouse IgG antibodies coupled to horse radish peroxidase were from Pierce. Glutathione-Sepharose and ECL reagent were from Amersham Pharmacia Biotech. Peptides: the 24 residue synthetic peptide whose sequence encompasses the PDK1 binding site termed PIFtide (REPRILSEEEQEMFRDFDYIADWC), the mutant D978A-PIFtide (numbering based on the human PRK2 sequence REPRILSEEEQEMFRDFAYLADWC), unrelated peptides (YRRAAVPPSPSLSRHSSPHQAEDEEE, and KKVKPPFIPTIRGREDVSNFDDEFT used in control experiments for FIG. 6), the PKB specific peptide substrate (RPRAATF), the PDK1 peptide substrates T308tide (KTFCGTPEYLAPEVRR), and PDKtide (KTFCGTPEYLAPEVRREPRILSEEEQEMFRDFDYLADWC) were synthesised by Dr G Blomberg (University of Bristol, UK).

General Methods

Molecular biology techniques were performed using standard protocols. Site directed mutagenesis was performed using QuikChange kit (Stratagene) following instructions provided by the manufacturer. DNA constructs used for transfection were purified from bacteria using Qiagen plasmid Mega kit according to the manufacturer's protocol, and their sequence verified using an automated DNA sequencer (Model 373, Applied Biosystems). Human kidney embryonic kidney 293 cells were cultured on 10 cm dishes in Dulbecco's Modified Eagle's medium containing 10% foetal bovine serum. Phospholipid vesicles containing phosphatidylicholine, phosphatidylserine and sn-1-stearoyl-2-arachidonoyl-D-PtdIns (3, 4, 5) P₃ [26] were prepared as described previously [13].

PDK1 Constructs

Full length PDK1 (residues 1-556), PDK1 (residues 52-556). PDK1 (residues 52-404), PDK1 (residues 1-360) and PDK1 (1-426) constructs were expressed in 293 cells with an N-terminal glutathione S-transferase (GST) tag from the pEBG2T vector [27] and affinity purified on glutathione-Sepharose [14]. The indicated Lys115, Ile119, Gin150 and Leu155 mutants of PDK1 used in this study were expressed and purified in a similar fashion. Between 0.5 and 1.0 mg of each GST-fusion protein was obtained by transfection of twenty 10 cm diameter dishes of 293 cells and each protein was more than 90% homogeneous as judged by SDS polyacrylamide gel electrophoresis (data not shown). PDK1 (residues 52-556) was also expressed in Sf9 cells with a hexahistidine (His) tag at the N-terminus and purified as described previously [24].

Yeast Two-Hybrid Screen

Mye-tagged human PDK1 was subcloned into the EcoRI/SalI site of pAS2-1 (Clonetech) as a Gal4 DNA binding domain fusion. A yeast two-hybrid screen was carried out by co-transforming pAS2-1 PDK1 and a pACT2 human brain cDNA library (Clontech) into the yeast strain Y190. Transformed yeast cells were incubated for 10 days at 30° C. on SD media supplemented with 25 mm 3-aminotriazole and lacking histidine, leucine and tryptophan. Approximately 5×10⁶ colonies were screened.

Yeast Two-Hybrid Analysis

Site directed mutants of pAS2-1 PDK1 (L155D), (L155E) and (L155S) were constructed. Y190 strain yeasts were co-transformed with the indicated combinations of vectors and grown on SD media lacking histidine, uracil, tryptophan and leucine at 30° C. until appearance of colonies. Yeast colonies were patched onto fresh media, incubated overnight at 30° C. and filter lifts taken. β-glactosidase activity was tested by incubating filters in X-Gal at 30° C. for 4 h.

Structural Modelling

The structure of the kinase domain of PDK1 (residues 92-341) was modelled using the programme Swiss-Pdb Viewer [hhtp://www.expasy.ch/spdbv/main page.htm. [28] connecting to Swiss Model Automated Protein Modelling Server. Modelling was based on several structures of the PKA catalytic subunit available in the database (Protein Data Bank Identification: 1YDR, 1CTP, 1STC, 1ATP and 1CDK). Sequence identity to PDK1 within the catalytic region (residues 55-297 of mouse PKA) was 40%, with a similarity of 68%.

Binding of PIF to Myc-PDK1

A pEBG2T plasmid encoding GST fused to the last 77 residues of PRK2 termed GST-PIF (10 μg) [24] and pCMV5 plasmid expressing Myc-PDK1 wild type or the indicated mutants of PDK1 (10 μg), were co-transfected into a 10 cm diameter dish of 293 cells using a modified calcium phosphate method [29]. 48 h post-transfection the cells were lysed in 0.6 ml of lysis buffer (50 mM Tris-HCl pH 7.5, 1 mM EGTA, 1 mM EDTA, 1% (by mass) Triton-X100, 1 mM sodium orthovanadate, 50 mM sodium fluoride, 5 mM sodium pyrophosphate, 0.27 M sucrose, 1 mM microcystin-LR, 0.1% (by vol) β-mercaptoethanol and one tablet of protease inhibitor cocktail per 50 ml of buffer) cleared by centrifugation, and 0.5 ml of supernatant was incubated for 2 h at 4° C. with 30 μl of glutathione-Sepharose. The beads were washed twice in lysis buffer containing 0.5 M NaCl, followed by two further washes in lysis buffer. The beads were resuspended in 1 vol of Buffer containing 100 mM Tris/HCl pH 6.8, 4% (by mass) SDS, 20% (by vol) glycerol and 200 mM DTT and subjected to SDS polyacrylamide gel electrophoresis. The gels were either stained with Coomassie blue, or analysed by immunoblotting with anti Myc antibodies.

Analysis of PIF-Binding to Myc-PDK1

Binding was analysed directly by surface plasmon resonance in an upgraded Bia-Lite™ system. PIFtide (comprising the last 24 residues of PRK2) was biotinylated though its C-terminal Cys and bound to an streptavidin-coated Sensor/Chip SA, as described previously [24]. Wild type or mutant preparations of GST-PDK1 (10-400 nM) were injected in an intracellular type buffer, over the immobilised biotinylated PIFtide at a flow rate of 30 μl per min as described previously (James et al (1996) Biochem J 315, 709-713). Alternatively, the wild type or mutant preparations of GST-PDK1 (1 μM) were incubated with PIFtide or D978A-PIFtide (0.10 μM) and the mixture injected over the immobilised peptides. The decrease is steady state binding between wild type and mutant GST-PDK1 and peptide was used to determine the K_{d} of interaction between PDK1 and the peptide. The decrease in the maximal response at different concentrations of peptide was used to evaluate the relative affinities of both peptides for PDK1. The sensor chip surface was regenerated by pulses of 10 mM NaOH.

Measurement of PDK1 Catalytic Activity

PDK1's ability to phosphorylate Thr308 of PKBα was measured using a mutant of GST-PKBα in which Ser473 was mutated to Asp (GST-473D PKBα) in the presence of phospholipid vesicles containing sn-1-stearoyl-2-arachidonoyl-D-PtdIns (3, 4, 5)P₃ [13]. The ability of wild type and mutant PDK1 to phosphorylate the synthetic peptides T308tide or PDKtide was carried out in 20 μl assays containing 50 mM Tris/HCl pH 7.5, 0.1% 2-mercaptoethanol 10 mM MgCl₂, 100 μM [γ³²P]ATP (^{˜}500 cpm/pmol) 0.5 μM microcystin-LR.PDK1 and the peptide concentrations indicated under Results. After incubation for 10 min at 30° C. the reaction was stopped by addition of 20 μl of 150 mM phosphoric acid. 35 μl of the resultant mixture was spotted into P81 phosphocellulose paper (2×2 cm) and the papers washed and analysed as described previously for assays of MAP kinase [30]. Wild type PIFtide or the mutant D978A PIFtide peptides were included in the reactions as indicated. Control assays were carried out in parallel in which either PDK1, or peptide substrate were omitted; these values were always less than 5% of the activity measured in the presence of these reagents. One Unit of PDK1 activity was defined as that amount required to catalyse the phosphorylation of 1 nmol of the T308tide in 1 min. The assays were linear with time up to a final PDK1 concentration of 5 U/ml.

Thermal Denaturation

Heat denaturation was performed by incubating the indicated forms of PDK1 (0.4 mg/ml) for 2 min at temperatures ranging from 30 to 65° C. The heat treatment was terminated by the addition of a 10-fold volume excess of ice cold buffer (50 mM Tris/HCl pH 7.5, 1 mM DTT and 0.1 mg/ml BSA), and the samples incubated for 2 min in an ice-water bath before a 4 μl aliquot was assayed for activity towards T308tide.

Results

PDK1 interacts with the C-terminal fragment of PKA. A yeast two-hybrid screen was carried out to identify proteins expressed in human brain that interact with PDK1. We identified a clone corresponding to the C-terminal 223 amino acids of PKA (termed PKA_{CT}) that yielded a positive interaction with full length PDK1 (FIG. 7A), but not with the PH domain of PDK1 (data not shown). PKA_{CT} includes part of the kinase domain as well as amino acids in the C-terminal non catalytic region of PKA that show high sequence homology between AGC subfamily kinases (FIG. 7B). The C-terminal 62 amino acids of PKA possesses significant homology with PIF and terminates in the sequence motif (347-Phe-Xaa-Xaa-PheCOOH). This sequence is similar to the PDK1 interacting motif in PIF (974Phe-Xaa-Xaa-Phe-Asp-Tyr979, numbering based on the human PRK2 sequence [24]) except that the Asp residue is replaced by the C-terminal carboxylate group of PKA and the C-terminal Tyr is missing. This suggested that the interaction of PKA_{CT} with PDK1 might be mediated by the C-terminal sequence 347-Phe-Xaa-Xaa-PheCOOH. The mutation of either or both of the C-terminal Phe347 and Phe350 to Ala of PKA_{CT} abolished its interaction with PDK1 (FIG. 7A), but the addition of four Gly residues to the C-terminus of PKA_{CT} to move the free carboxylate group to another position had no effect on the ability of PKA_{CT} to interact with PDK1 (FIG. 7A). These findings indicate that both Phe residues but not the carboxylate group in this motif are required for the interaction of PKA_{CT} with PDK1.

Identification of a Putative Hydrophobic Pocket in the Kinase Domain of PDK1 that Interacts with PIF

PKA was the first protein whose 3-dimensional structure was solved at high resolution [31] and has established a structural framework for the catalytic domain of most protein kinases [reviewed in 32]. Analysis of the structure of PKA revealed that the non catalytic C-terminus forms a loop that interacts with the kinase domain (FIG. 8A). Most interestingly, the C-terminal residues of PKA implicated above in binding to the kinase domain of PDK1, interact with a deep hydrophobic pocket in the small lobe of the PKA catalytic domain (FIG. 8B). This site does not overlap with the ATP or peptide substrate binding sites on PKA. The residues that make obvious hydrophobic interactions with the two Phe residues in the terminal 347Phe-Xaa-Xaa-Phe motif of PKA are Lys76, Val80, Lys111 and Leu116 of PKA (FIG. 2B).

A sequence alignment of the kinase domains of PDK1 and PKA indicated that the residues equivalent to Lys76 (Lys115 on PDK1) and Leu116 (Leu155 on PDK1) of PKA are conserved in PDK1 (FIG. 8D). Molecular modelling of the structure of the kinase domain of PDK1 based on that of PKA confirmed that PDK1 is likely to possess a hydrophobic pocket in the equivalent region of its kinase domain and that Lys115 and Leu155 in PDK1, are likely to lie in positions equivalent to Lys76 and Leu116 in PKA. The residues on PKA equivalent to Val80 and Lys111 which form part of the hydrophobic pocket lie in the same position as Ile119 and Gln150, respectively of the PDK1 kinase domain. The model of the PDK1 kinase domain indicates that these residues, as well as Lys115 and Leu155 may form part of a hydrophobic binding site (FIG. 8D).

Effect of Mutation of Lys115 and Leu155 on PIF-Binding to PDK1

The model for the hydrophobic pocket in PDK1 predicts that Lys115 and Leu155 should participate in a hydrophobic interaction with the residues equivalent to Phe974 and Phe977 of PIF. We therefore mutated Lys115 to Ala and Leu155 to Ser, Asp or Glu and compared the ability of these PDK1 mutants and wild type PDK1 to interact with GST-PIF (FIG. 9). As reported previously, a complex was readily observed between GST-PIF and wild type PDK1. In contrast, the K115A interacted very poorly with PIF, whilst none of the L155 mutants interacted significantly with PIF, although these PDK1 mutants were expressed to the same level as wild type PDK1 (FIGS. 9C and 9G).

Surface Plasmon Resonance (SPR) measurements confirmed a high affinity interaction between wild type GST-PDK1 and immobilised, biotinylated synthetic peptide termed PIFtide, whose sequence encompasses the PDK1 binding site, as reported previously [24]. However the L155S, L155D and L155E mutants of PDK1 had no detectable affinity for PIFtide (FIG. 9D), whilst the K115A interacted weakly with PIFtide (FIG. 9D).

A yeast 2 hybrid screen also confirmed that the L155S, L155D, and L155E mutants of PDK1, failed to interact with PIF (FIG. 10). Furthermore, the interaction of PKA_{CT} with the L155S, L155D or L155E mutants of PDK1 was greatly reduced in a yeast 2 hybrid screen, further suggesting that the carboxyl terminus of PKA interacts with the PDK1 catalytic domain at the same site as PIF (FIG. 10).

The K115A, L155S, L155D and L155E mutants of PDK1 were 50-60% as efficient as wild type PDK1 in activating GST473D-PKBα in the presence of MgATP and PtdIns (3, 4, 5)P₃ (FIG. 11). This indicated that the conformation of the active site of PDK1 was not significantly impaired by these mutations.

Effect of Mutation of Ile119 and Gln150 on PIF-Binding to PDK1

Ile119 and Gln 150, which are also predicted to form part of the PIF-binding pocket on the small lobe of the PDK1 kinase domain were mutated to Ala. In both pull down (FIGS. 9E and 9F) and Surface Plasmon Resonance experiments (FIG. 9H) the I119A and Q150A mutants of PDK1 interacted very weakly with PIF compared to wild type PDK1. These mutants also activated a GST-473D-PKBα at 60-70% of the rate of wild type PDK1 (data not shown).

Effect of PIF on the Catalytic Activity of PDK1 Towards a Peptide Substrate

A recent study by Dong and colleagues [27] demonstrated that PDK1 phosphorylates a synthetic peptide KT*FCGTPEYLAPEV-RR, here termed T308tide, whose sequence encompasses residues 307 to 320 of PKBα with 2 Arg residues added to the C-terminus to make the peptide bind to P81 paper. As it is unlikely that T308tide would interact with the PIF-binding pocket of PDK1, we decided to use this substrate to investigate the effect of PIF-binding on the catalytic activity of PDK1. We confirmed that T308tide was phosphorylated in vitro by PDK1 although the Kₘ was very high (>10 mM). We also established that T308tide was phosphorylated at the residue equivalent to Thr308 of PKBA (indicated by an asterisk), by solid phase sequencing of ³²P-labelled T308tide phosphorylated by PDK1 (data not shown). PDK1 activity towards T308tide was increased up to 4-fold in the presence of PIFtide. The concentration required for half-maximal activation was 0.14 μM (FIG. 11A) which correlates with the affinity of PDK1 for PIFtide (K_{d} of ^{˜}0.3 μM [24]). This increase in PDK1 activity was observed with either full length PDK1 or forms lacking the N-terminal or C-terminal non-catalytic regions (data not shown). The effects of PIFtide on PDK1 activity were unaffected by preincubating these components for up to 30 min on ice prior to initiating the assay. Similarly, a mutant D978A-PIFtide, which exhibits a 10-fold reduced affinity for PDK1 [24], was 8-fold less effective at inducing PDK1 activation (FIG. 12A). Several unrelated peptides of similar size were unable to induce any activation of PDK1 (data not shown). This strongly indicates that PDK1 is activated directly by PIF. Furthermore, PIF did not alter the Kₘ of PDK1 for ATP (data not shown).

GST-PDK1 activity was reduced by 50% if the enzyme was heated for 2 min at 50° C. (TM50 value, FIG. 12B). However, PDK1 was stabilised in the presence of wild type PIFtide, the TM₅₀ being increased by 8-10° C. PIF also caused a 6-10° C. increase in the TM₅₀ value for all GST-PDK1 transcription mutants tested which either lack the PH domain, the N-terminal 51 residues or both non-catalytic domains (data not shown). The L155D mutant of GST-PDK1 was more heat labile than wild type PDK1 with a TM₅₀ value of 42° C. As expected, PIF did not significantly stabilise this mutant (FIG. 12B).

Activity of PDK1 Mutants Towards T308tide

We next tested the specific activities of the PIF-binding pocket mutants towards T308tide. The L115A, L155S, L155D, and L155E mutants of PDK1 phosphorylated T308tide 3 to 5-fold more rapidly than wild type PDK1, ie at a rate similar to that of wild type PDK1 in the presence of PIF (FIG. 7). PIFtide did not further activate these mutants consistent with their inability to bind PIF. In contrast, the L119A and Q150A mutants of PDK1 possessed a specific activity similar to wild type PDK1 and were stimulated −2-fold in the presence of PIF. However, 10-fold more PIF was required for maximal activation compared to wild type PDK1, consistent with the reduced affinity of these mutants for PIF (FIG. 9).

PDKtide is a Vastly Superior Peptide Substrate for PDK1

The results presented above suggested that a peptide substrate for PDK1 might be phosphorylated with a much lower Kₘ value if it also contained the PDK1 interacting sequence of PIF. We therefore synthesised a 39 amino acid polypeptide composed of T308tide fused to PIFtide, and termed it PDKtide. This peptide was a vastly superior substrate for PDK1 than T308tide; its Kₘ was −80 μM (compared to >10 mM for T308tide) and when assayed at 100 μM, PDKtide was phosphorylated at a rate over 100-fold greater than that using T308tide (FIG. 14A). The activity of PDK1 towards PDKtide was inhibited by inclusion of PIFtide in the assay, in contrast to T308tide phosphorylation which was stimulated by PIFtide (FIG. 14B).

Discussion

The C-terminal residues of PKA, Phe-Xaa-Xaa-PheCOOH, correspond to part of the PDK1 binding motif of PIF. These residues are known to interact with the small lobe of the kinase domain of PKA at a location distinct from the ATP or peptide substrate binding sites (FIG. 2). In this paper we demonstrate that PKA_{CT} also interacts with the kinase domain of PDK1 in a yeast 2 hybrid screen, and that mutation to Ala of the residues in PKA_{CT} equivalent to Phe347 or Phe350 abolishes/significantly reduces its interaction with PDK1 (FIG. 7). As the mutation of the equivalent Phe residues to Ala on PIF also abolishes its interaction with PDK1[24], these findings suggested that the PKA_{CT} and PIF might interact at the same site in the PDK1 kinase domain.

The residues in the kinase domain of PKA known to interact with the C-terminus of this protein are present in PDK1, and their mutation either abolished or significantly diminished the interaction of PDK1 with both PIF and PKA_{CT}. These observations strongly suggest that PDK1 possesses an equivalent hydrophobic pocket in its kinase domain that interacts with PIF and PKA_{CT}. PDK1 is itself a member of the AGC subfamily of protein kinases but, in contrast to PKA, it does not possess a hydrophobic Phe-Xaa-Xaa-Phe-motif at the equivalent position. PDK1 is therefore likely to possess an unoccupied PIF-binding pocket in its kinase domain which is available to interact with the C-terminal hydrophobic motifs of PKA and other AGC subfamily members.

The interaction of PIF with PDK1 converts it from an enzyme that only phosphorylates PKBα at Thr308 to a form that phosphorylates both Thr308 and Ser 473 in a PtdInds (3, 4, 5)P₃ or PtdIns (3, 4) P₂ dependent manner [24]. The PDK1 binding motif in PIF (Phe-Xaa-Xaa-Phe-Asp-Tyr) could therefore be required as a pseudosubstrate sequence raising the possibility that PIF interacts with the substrate binding site of PDK1. However, if this were the case PT would be expected to prevent PDK1 from phosphorylating PKBα at Ser473 rather than promoting this reaction. The finding that PIF interacts with a site on the kinase domain of PDK1 which is distinct from the substrate binding site explains why this is not the case, and suggests that PIF may be capable of inducing conformational changes in the PDK1 catalytic core which alter its substrate specifically.

In order to assess the effect of PIF on the intrinsic catalytic activity of PDK1 we used the peptide substrate T308tide rather than a protein substrate of PDK1 such as p70 S6 kinase which may interact with PDK1 at a site that overlaps the PIF-binding pocket (see Example 1). Using this assay, we demonstrated that the PIF-binding pocket was likely to be important in regulating the activity of PDK1. When unoccupied, the PIF-binding pocket appears to suppress the activity of PDK1, because the mutation of key residues that form it, Lys115 and Leu155 enhanced PDK1 activity towards T308tide to the level equivalent to that of wild type PDK1 in the presence of PIF (FIG. 6). It is therefore likely that the binding of PIF transduces an allosteric transition which stabilises a functionally active conformation of PDK1.

The interaction of PIF with PDK1 requires an Asp residue (Asp978) at the position equivalent to Ser473 of PKBα. An interesting possibility was that the C-terminal carboxylate group of the Phe-Xaa-Xaa-PheCOOH motif of PKA_{CT} may have played an analogous function to Asp978 of PIF to enable binding to PDK1. However, this does not seem to be the case as the addition of four glycines to the C-terminus of PKA_{CT} did not affect its interaction with PDK1. The C-terminal carboxylate group of Phe350 of PKA does not form any interaction with the hydrophobic pocket on the kinase domain of PKA but instead faces outwards from this site and forms a hydrogen bond with Gln35 in the N-terminal non-catalytic region of PKA [33]. The importance of this interaction has not yet been investigated by mutating Gln35 of PKA. Similarly, it is possible that Asp978 of PIF may not interact with the PIF binding pocket, but to a distinct region of PDK1.

Sequence alignment of PKB, SGK and p70 S6 kinase indicates that these members of the AGC subfamily of kinases are also likely to possess a PIF-binding pocket in their kinase domains. These kinases are all activated by phosphorylation of a Ser/Thr residue at the position equivalent to Asp978 of PIF. It is therefore possible that the introduction of a negative charge at this site by phosphorylation causes the residue of this motif to interact with their own PIF-like binding pockets thereby leading to increased activity and stability, in a similar manner to the way in which PIF activates and stabilises PDK1. The observation that phosphorylation of the same site increases the stability of conventional PKC isoforms is consistent with this consensus[8].

The PIF-binding pocket may be the site that enables PDK1 to interact with its substrates. This interaction may also induce a conformational change which enhances the rate at which these substrates are phosphorylated by PDK1. For example, the interaction of PKA with PDK1 via the C-terminal Phe-Xaa-Xaa-PheCOOH motif of PKA may facilitate the phosphorylation of PKA at Thr197. However, we have recently shown that PDK1 is unable to interact with or phosphorylate p70 S6 kinase in the presence of PIF [25] and this is also true for SGK, PRK2 and PKCζ (data not shown). This suggests that p70 S6 kinase and SGK may require to interact with PDK1 at a site that overlaps with the PIF-binding pocket in order to become phosphorylated [25]. P70S6 kinase when phosphorylated at its hydrophobic motif interacted with PDK1 with much higher affinity. PKCζ is another protein kinase-that interacts with PDK1 [17, 18], which, like PRK1, PRK2 and PKCα, possesses an acidic residue rather than a Ser/Thr in the C-terminal hydrophobic motif. It is therefore likely that this region of PKCζ interacts with the PIF-binding pocket of PDK1, and this interaction enables PDK1 to phosphorylate and hence to activate PKCζ. This is shown by Balendran et al (2000) J Biol Chem 275(27), 20806-20813 and discussed in Biondi et al (2000) EMBO J 19(5), 979-988 (both specifically incorporated herein by reference). Thus, the C-termini of these kinases may be acting as PDK1 “docking sites”. Consistent with the Phe-Xaa-Xaa-Phe-Asp-Tyr motif of PIF being a docking site for PDK1 the addition of this motif to T308tide greatly increases the rate at which it is phosphorylated by PDK1 (FIG. 14) PRK1, PRK2, PKCζ, and PKC₁ may have another role, to allow PDK1 to phosphorylate PKB and other members of the AGC subfamily at the site equivalent to Ser473 on PKB [12, 24].

In summary, PDK1 appears to possess a hydrophobic binding site in the small lobe of the kinase catalytic domain which regulates its activity as well as its interaction with substrates. These findings raise the possibility of developing novel drugs that interact with the PIF-binding pocket on PDK1. Such drugs could either activate or inhibit PDK1 by modulating its interaction with particular substrates, and thus could switch on or switch off signal transduction pathways that are regulated by PDK1. Thus T308tide could be used as a substrate to identify compounds that activate PDK1 by mimicking the effect of PIF, while PDKtide may be the peptide of choice to identify compounds that disrupt the interaction of PDK1 with PIF.

References

1. Leevers et al (1999) Curr Opin Biol 11, 219-225

2. Shepherd et al (1998) Biochem J 333: 471-479

3. Alessi D R and Downes C P (1998) Biochem Biophys Acta 1436, 151-164

4. Proud C G (1995) Trends in Biochem Sci 21, 181-185

5. Pullen N & Thomas G, (1998) FEBS LETT 410, 78-82

6. Kobayashi T & Cohen P (1999) Biochem J 339, 319-328

7. Park et al (1999) EMBO J18, 3024-3033

8. Mellor H and Parker P J, (1998), Biochem J, 332, 281-292

9. Alessi, D R and Cohen P (1998) Curr Opin Genet Dev 8, 55-62

10. Pearson et al (1995) EMBO J 14, 5278-5287

11. Belham et al (1999) Current Biol 9, R93-R96.

12. Peterson, R T & Schreiber, S L (1999) Current Biol 9, R521-524

13. Alessi et al (1997) Curr Biol 7, 261-269

14. Alessi et al (1997) Curr Biol 7, 776-789

15. Stokoe et al (1997) Science 277, 567-570

16. Stephens et al (1998) Science 279, 710-714

17. LeGood et al (1998) Science, 281, 2042-2045

18. Chou et al (1998) Curr Biol, 8, 1069-1077

19. Dutil et al (1998) Curr Biol, , 8, 1366-1375

20. Alessi et al (1998) Curr Biol 8, 69-81

21. Pullen et al (1998) Science, 279, 707-710

22. Chen et al (1998) Proc Natl Acad, USA 95, 9849-9854

23. Etchebehere et al (1997) Eur J Biochem, 248, 820-826

24. Balendran et al (1999) Current Biology 9, 393-404

26. Gaffney P R J & Reese C B (1997) Bioorg Med Chem Lett, 7, 3171-3176

27. Sanchez et al (1994) Nature, 372, 794-798

28. Guex N and Peitsch M C (1997) Electrophoresis, 18, 2714-2723

29. Alessi et al (1996) EMBO J 15, 6541-6551

30. Alessi et al (1994), Methods of Enzymology 255, 279-290

31. Knighton et al (1991) Science 253, 407-414

32. Taylor, S S & Radzio-Andzelm E (1994) Structure 2, 345-355

33. Zheng et al (1993) Biochemistry 32, 2154-61

34. Holland P M and Cooper J A (1999) Curr Biol 9, R329-R331

35. Jacobs et al (1999) Genes and Development, 10, 163-175

### EXAMPLE 3

### PDK1 Hydrophobic PIF Pocket is Essential for Phosphorylation and Activation of S6K and SGK but Not PKB

In this example we demonstrate that the PIF binding pocket of PDK1 plays a key role in enabling PDK1 to phosphorylate and activate p70 ribosomal S6 kinase (S6K)[6,7] S6K1, serum and glucocorticoid induced kinase-1 (SGK)[8-10] SGK1 and mutant of PKBα that lacks its PH domain (ΔPH-PKBα). We also demonstrate that the hydrophobic motif of S6K1, SGK1 and ΔPH-PKBα plays a key role in allowing the kinases to become phosphorylated by PDK1 in vitro and in vivo. In contrast neither the PIF binding pocket of PDK1 or the hydrophobic motif of PKBα are required for the phosphorylation of PKBα by PDK1, in the presence of phosphatidylinositol(3,4,5)P₃. We also provide evidence that non-phosphorylated forms S6K1 and SGK1 which are poor substrates for PDK1 do not interact with PDK1. Removal of the C-terminal autoinhibitory domain of S6K1 enables PDK1 to interact and phosphorylate S6K1. A mutation of SGK1 that mimics phosphorylation at its hydrophobic motif, also enables PDK1 to interact and phosphorylate it. We suggest a model by which phosphorylation of PDK1 substrates thus far been identified other than PKB are regulated by the direct interaction of their hydrophobic motif with the PIF binding pocket of PDK1.

PKB is activated usually within 2 minutes of a cell being stimulated with insulin and growth factors [11-13]. It possesses an N-terminal plekstrin homology (PH) domain that interacts with PtdIns(3,4,5)P₃/PtdIns(3,4)P₂ resulting in the recruitment of PKB to the plasma membrane where it becomes activated by the phosphorylation of 2 residues. One lies in the T-loop of the kinase domain (Thr308 in PKBα) and the other is located C-terminal to the catalytic domain, in a region termed the “hydrophobic motif” (Ser473 in PKBα) [14]. S6K [6] and SGK [8-10] also possess residues equivalent to Thr308 (Thr252 in S6K1 and Thr256 in SGK1) and Ser473 (Thr412 in S6K1 and Thr422 in SGK1) whose phosphorylation is required for activation of these kinases in vivo. The phosphorylation S6K and SGK at both its T-loop and hydrophobic motif like that of PKB, is dependent upon PI 3-kinase activation. In contrast to PKB, S6K and SGK do not possess a PH domain and do not interact with PtdIns(3,4,5)P₃/PtdIns(3,4)P₂. S6K and SGK are also activated markedly slower than PKB□ following cell stimulation, with maximal activation occurring after 10-40 minutes [9, 10, 12].

PKB, S6K1 and SGK are phosphorylated at their T-loop by the 3-phosphoinositide-dependent protein kinase1 (PDK1) [14]. This enzyme is also an AGC family member, and possess a PtdIns(3,4,5)P₃/PtdIns(3,4)P₂ binding PH domain C-terminal to the catalytic domain. Following, PI 3-kinase activation, PDK1 and PKB are thought to co-localise at the plasma membrane through their interactions with PtdIns(3,4,5)P₃/PtdIns(3,4)P₂. In addition to recruiting PKB to membranes of cells the binding of PtdIns(3,4,5)P₃/PtdIns(3,4)P₂ to the PH domain of PKB may induce a conformational change that enables PDK1 to phosphorylate it [14]. As S6K and SGK do not interact with PtdIns(3,4,5)P₃/PtdIns(3,4)P₂, nor is the rate at which these are phosphorylated by PDK1 in vitro enhanced in the presence of PtdIns(3,4,5)P₃/PtdIns(3,4)P₂ [9, 15], the mechanism by which activation of PI 3-kinases induces activation of S6K and SGK must be distinct from PKB.

The kinase domain of PDK1 was found in a yeast 2 hybrid screen to interact with a region of the protein kinase C-related kinase-2 (PRK2), termed the PDK1 Interacting Fragment (PIF) [16]. PIF is situated C-terminal to the kinase domain of PRK2, and contains a hydrophobic motif (Phe-Xaa-Xaa-Phe-Asp-Tyr), similar to that found in PKBα(Phe-Xaa-Xaa-Phe-Ser-Tyr), except that the residue equivalent to Ser473 is Asp. Mutation of the conserved aromatic residues in the hydrophobic motif of PIF or mutation of the Asp residue to either Ala or Ser prevented the interaction of PIF with PDK1 [16].

Subsequent work demonstrated that a 24 amino acid fragment of PIF (termed PIFtide), that encompasses the hydrophobic motif of PRK2 bound to a hydrophobic pocket on the small lobe of the PDK1 kinase domain which was termed, the “PIF binding pocket” [17]. Three lines of evidence indicate that this interaction could serve as a “docking site”, enabling the recruitment of PDK1 to PRK2 which is essential for its phosphorylation by PDK1. Firstly, a PDK1 mutant in which the central residue (Leu155) in the PIF-binding pocket is mutated so that PDK1 can not interact with PIF [17] possessed greatly reduced affinity for PRK2 [18]. Secondly, overexpression of PIF in 293 cells prevented the phosphorylation of PRK2 at its T-loop residue. Thirdly, mutation of a conserved Phe to Ala on the hydrophobic motif of PRK2 greatly reduced the affinity of PRK2 for PDK1 and furthermore, this mutant was not phosphorylated at its T-loop residue when expressed in cells [18]. Similar findings were made for another PDK1 substrate namely, protein kinase Cζ (PKCζ), which is similar in structure to PRK2 and also possesses a acidic residue in its hydrophobic motif at the residue equivalent to Ser473 of PKBα[18].

In addition it is likely that the interaction of PDK1 with the hydrophobic motif of PRK2 and PKCζ will directly activate PDK1, as PIFtide increased 4-fold the rate at which PDK1 phosphorylated a peptide substrate that is derived from the T-loop of PKBα (T308tide) [17]. Furthermore, T308tide is a very poor substrate for PDK1, but if it is fused to PIFtide (PDKtide) it becomes a vastly superior substrate [17]. Recently, Frodin and colleagues [19] have also demonstrated that PDK1 interacts with another AGC kinase substrates termed p90RSK only when it is phosphorylated at its hydrophobic motif and present evidence that this interaction recruits PDK1 to p90RSK and may also activate PDK1.

Here we investigated the role of the hydrophobic PIF binding pocket on PDK1, in enabling PDK1 to phosphorylate and activate 3 of its AGC kinase substrates that are activated in response to insulin, namely, S6K1, SGK1, PKB as well as a mutant of PKBα that lacks its PH domain (ΔPH-PKBα) which like S6K and SGK does not interact with PtdIns(3,4,5)P₃/PtdIns(3,4)P₂. Our data indicate that the PIF binding pocket of PDK1 plays a critical role in enabling PDK1 to phosphorylate and activate S6K1, SGK1 and ΔPH-PKBα but not wild type PKBα. Our results suggest model in the phosphorylation of PDK1 substrates other than PKB, would be regulated by the ability of the hydrophobic motif of these substrates to interact with PDK1.

Materials and Methods

Materials. Complete protease inhibitor cocktail tablets and anti-Myc monoclonal antibodies were from Roche; tissue culture reagents and microcystin-LR were from Life Technologies; glutathione-Sepharose and ECL reagent were form Amersham Pharmacia Biotech. Precast gradient SDS polyacrylamide gels were from invitrogen.

Antibodies. The characterisation of the phospho-specific antibodies recognising SGK phosphorylated at its T-loop (Thr256) termed T256-P has been described previously□. The phospho-specific antibody recognising PKBα phosphorylated at Thr308 (termed T308-P) was raised in sheep against the peptide KDGATMKTFCGTP (corresponding to residues 301 to 313 of the human PKBα), in which the underlined residue is phosphothreonine. The antibody recognising S6K1 phosphorylated at Thr229 was raised in sheep against the peptide HDGTVTHTFCGTIEY (corresponding to residues 245 to 259 of long splice variant of human S6K1) in which the underlined residue is phosphothreonine. The antibodies were affinity purified on CH-Sepharose covalently coupled to the phosphorylated peptide, then passed through a column of CH-Sepharose coupled to the non-phosphorylated peptide. Antibodies that did not bind to the latter column were selected. Monoclonal antibody recognising the Myc epitope was from Roche, the monoclonal antibodies recognising GST and the FLAG epitope were purchased from Sigma. Horse radish peroxidase conjugated secondary antibodies were from Pierce.

General methods. Molecular biology techniques were performed using standard protocols. Site directed mutagenesis was performed using a QuickChange kit (Stratagene) following instructions provided by the manufacturer. DNA constructs used for transfection were purified from bacteria using Qiagen plasmid Mega kit according to the maufacturer's protocol, and their sequence verified. Human kidney embryonic 293 cells were cultured on 10 cm diameter dishes in Dulbecco's modified Eagle's medium containing 10% foetal bovine serum. Transfections were performed using a modified calcium phosphate method and 10 μg of each plasmid per dish. Phospholipid visicles containing phosphatidiylcholine, phosphatidylserine and sn-1-stearoyl-2-arachidonoyl-D-PtdIns(3,4,5)P₃ [20] were prepared as previously described [21].

Buffers. Buffer A: 50 mM Tris-HCl pH 7.5, 1 mM EGTA, 1 mM EDTA, 1% (by mass) Triton-X 100, 1 mM sodium orthovanadate, 50 mM sodium fluoride, 5 mM sodium pyrophosphate, 0.27 M sucrose, 1 μM microcystin-LR, 0.1% (by vol) β-mercaptoethanol and ‘complete’ proteinase inhibitor cocktail (one tablet per 25 ml, Roche). Buffer B: 50 mM Tris/HCl pH 7.5, 0.1 mM EGTA, 10 mM β-mercaptoethanol and 0.27M sucrose.

Protein expression and purification. Wild type-PDK1 [22], PDK1[L155E], PDK1[K115A], PDK1[I119A], PDK1[Q150A] [17], and PDK1[L155A] [18], in the pEBG2T vector was used to express the wild type and indicated mutants of PDK1 fused through their N-terminus to glutathione S-transferase (GST). Wild type PDK1 [22] and mutant PDK1[L155E] [17] in the pCMV5 vector was used to express these proteins with an N-terminal Myc tag.

All S6K1, SGK1 and PKBα substrates employed in this study are illustrated in FIG. 17. All S6K1 mutants lacking the C-terminal 104 residues are termed S6K1-T2. N-terminal Flag epitope tagged S6K1, S6K1-T2, S6K1[T412E], S6K1-T2[T412E] pCMT2-T2-S6K1[T412E] were expressed in the pCMT2 vector [23]. N-terminal GST tagged S6K1, S6K1-T2 [9], S6K1-T2[F411A] were expressed in the pEBG2T vector.

All SGK1 mutants expressed in this study lack the N-terminal 60 amino acids. N-terminal HA epitope tagged SGK1, SGK1[T422E] [9], SGK1[F421A] were expressed in the pEBG2T vector.

N-terminal GST-tagged PKBα [21], PKBα [S473D] [16], PKBα [F472A], ΔPH-PKBα [22], ΔPH-PKBα[S473D], ΔPH-PKBα[F472A] were expressed in the pEBG2T vector.

The GST fusion proteins were expressed in human embryonic kidney 293 cells. For the expression of each construct, twenty 10 cm diameter dishes of 293 cells were cultured and each dish transfected with 10 μg of the pEBG-2T construct, using a modified calcium phosphate method [24]. 24 h post-transfection, the cells were deprived of serum for 16 h and then lysed in 0.6 ml of ice-cold Buffer A, the lysates pooled, centrifuged at 4° C. for 10 min at 13, 000×g and the GST-fusion proteins were purified by affinity chromatography on glutathione-Sepharose and eluted in 50 mM Tris pH 7.5, 0.1 mMEGTA, 0.27 M Sucrose, 0.1% (by vol) 2-mercaptoethanol and 20 mM glutathione as described previously [21]. Typically between 0.3 and 1.0 mg of each GST-fusion protein was obtained and each protein was more than 75% homogeneous as judged by SDS polyacrylamide gel electrophoresis (data not shown).

SGK1[T422E] when expressed in 293 cells is phosphorylated at Thr256 [9] and the purified GST-SGK1[T412E] was dephosphorylated by incubation with PP2A 30 mU/ml at 30° C. for one hour and the reaction was terminated by the addition of microcystin-LR (1 μM) the samples were left at 30° C. for a further 5 min and frozen in liquid nitrogen and stored at −80° C. until required. Although, GST-SGK1 is not phosphorylated at Thr256, this was also subjected to treatment with PP2A to enable comparsion of phosphorylation of SGK1 and SGK1[T422E]. S6K1-T2 and S6K1-T2[T412E] were also expressed as His-tag proteins in a bacuolovirus/insect cell expression system and purified by nickel agarose affinity chromatography as described previously[25]. S6K1-T2[T412E] expressed in this manner is not phosphorylated at Thr252.

Phosphorylation of AGC kinase substrates by PDK1. The phosphorylation of PDK1 substrates was performed in a final volume of 20 μl in a buffer containing 50 mM Tris-HCl pH 7.5, 0.1% (by vol) 2-mercaptoethanol, 10 mM magnesium chloride, 100 μM [γ-³²P]ATP (^{˜}1000 c.p.m./pmol), 0.5 μM microcystin-LR, 0.6 μM AGC kinase substrate and 0.6 to 30 nM wild type PDK1 or the indicated mutant of PDK1. After 10 min the reactions were stopped by addition of Laemmli Sample Buffer (100 mM Tris-HCl pH 6.8, 4% (by mass) SDS, 20% (by volume) glycerol and 200 mM dithiothreitol (DTT), boiled, and the samples subjected to separation by SDS-polyacrylamide gel electrophoresis. The gels were exposed and analysed with a Fuji PhosphoImager known amounts of [γ-³²P]ATP spotted onto a blank gels to permit quantification of the data. The experiments were performed so that the amount of PDK1 did not phosphorylate more than 20% of the substrate. Control in which PDK1 was omitted from the reaction was taken as the blank value.

Activation of AGC kinase PDK1 substrates. Phosphorylation reactions were carried out as above except that non-radioactive ATP replaced [γ-³²P]ATP. Following 10 min at 30° C., cocktail (30 μl) containing 50 mM Tris-HCl pH 7.5, 0.1% (by vol) 2-mercaptoethanol, 10 mM magnesium chloride, 100 μM [γ-³²P]ATP (^{˜}1000 c.p.m./pmol), 0.5 μM microcystin-LR and 100 μM peptide substrate crosstide (GRPRTSSFAEG) and [γ-³²P]ATP (^{˜}600 c.p.m./pmol). Reactions were stopped by the addition of 25 μl of 0.2 M EDTA pH 8.0, spotted onto P81 phosphocellulose paper, washed and analysed as described for the assay of MAP kinase [26]. The amount of PDK1 was in the assay was varied so that the assay was in the linear range. One unit of activity is deficed as phosphorylation 1 nmol of substrate in 1 min.

Binding of PDK1 to SGK1 and S6K1. For the data presented in FIG. 22, 293 cells were cotransfected with 10 μg of the wild type or mutant PDK1 plasmid and 10 μg of either the wild type or mutant S6K1 or SGK1. 36 h post-transfection the cells were lysed in 0.6 ml of Buffer A and the lysates were cleared by centrifugation at 13 000×g for 10 min at 2° C., and 0.5 ml of supernatant was incubated for 2 h at 4° C. with 30 μl of glutathione-Sepharose. The beads were washed twice in Buffer A containing 0.5 M NaCl, followed by two further washes in Buffer A. The beads were resuspended in 30 μl Laemmli Sample Buffer and subjected to SDS polyacrylamide gel electrophoresis. The gels were either stained with Coomassie blue, or analysed by immunoblotting with either anti-Flag or anti-Myc antibodies (described below).

Immunoblotting. For the Myc and Flag blots of cell lysates 5 μg of protein was used. Immunoblotting with the phosphospecific antibodies (0. 5-2 μg/ml) in the presence of 10 μg/ml dephospho peptide corresponding to the antigen used to raise the antibody in 50 mM Tris/HCl pH 7.5, 0.15M NaCl, 0.5% (by vol) Tween (TBS-Tween) containing in 50 mM Tris/HCl pH 7.5, 0.15M NaCl, 0.5% (by vol) Tween (TBS-Tween) 5% (by mass) skimmed milk. Detection was performed using horse radish peroxidase conjugated secondary antibodies and the enhanced chemiluminescence reagent. (Amersham/Pharamcia).

For the T816-P blots 25 μg of cell lysate protein was used. For the T410-P blots, 150 μg of cell lysate protein was immunoprecipitated using 5 μl of Flag affinity gel and washed as described above. Cell lysates or immunoprecipitates were made 1% in SDS, subjected to SDS/polyacrylamide gel electrophoresis, and transferred to nitrocellulose. The nitrocellulose membranes were immunoblotted using either the anti-Myc (0.4 μg/ml ), anti-Flag antibodies (0.4 μg/ml) and 10% (by mass) skimmed milk.

Results

All the wild type and mutant forms of AGC kinase substrates employed in this study are defined in FIG. 17. Unless stated otherwise the form of S6K used in this study lacks the C-terminal 104 residues as full length S6K1 is not an efficient substrate for PDK1 in vitro [15, 27]. The form of SGK1 used in this study lacks the N-terminal 61 amino acids as full length SGK1 protein is unstable and can not be expressed at significant levels [9].

Role of PDK1 “PIF-Pocket” on phosphorylation and activation of S6K1, SGK1 and PKB by PDK1. We first investigated the role of the hydrophobic PIF binding pocket on PDK1, in enabling PDK1 to phosphorylate and activate 3 of its AGC kinase substrates that are activated in response to insulin, namely, S6K1, SGK1 and PKB. We initially tested whether a PDK1 mutant (PDK1[L155E]) in which the hydrophobic PIF binding pocket has been disrupted [17] could phosphorylate and activate these AGC kinase substrates. Strikingly, the phosphorylation of S6K1 and SGK1 by PDK1[L155E] was drastically reduced compared to wild type PDK1 (FIG. 18 and Table 1). We also employed mutants of S6K1 (S6K1[T422E]) and SGK1 (SGK1[T422D]) in which their hydrophobic motif phosphorylation site was changed to an acidic residue which significantly increases the rate at which these are phosphorylated and activated by PDK1 (Table 3 and [9, 15, 27]). We found that both S6K1[T422E] and SGK1[T422D] were also very poorly phosphorylated and activated by PDK1[L155E] compared to wild type PDK1 (FIG. 18). In contrast, PKBα and its acidic hydrophobic motif mutant, PKBα[S473D] were equally good substrates for wild type PDK1 and PDK1[L155E]. Interestingly however, mutant forms of PKBα and PKBα[S473D] that lack the PH domain (ΔPH-PKBα and ΔPH-PKBα[S473D]) and are thus more similar in structure to S6K1 and SGK1 (FIG. 17) are very poor substrates for PDK1[L155E] compared to wild type PDK1. ΔPH-PKBα is phosphorylated by PDK1 at a 50-100 fold lower rate than full length PKBα (Table 3) and its phosphorylation, like that of S6K1 and SGK1 by PDK1, is not influenced by PtdIns(3,4,5)P₃[9, 15]. It should be noted however, that ΔPH-PKBα is phosphorylated by PDK1 at a 10-fold lower initial rate than S6K1 and SGK1 and ΔPH-PKBα[S473D] is phosphorylated at ^{˜}100-fold lower rate than S6K1[T412E] and SGK1[T422E] (Table 3).

TABLE 3
Relative phosphorylation of PDK1 substrates

| | |
|---|---|
| PDK1-wt | PDK1-L155E |

| | − | +PIFtide | − | +PIFtide |
|---|---|---|---|---|
| P70 S6K-T2 | 10.4 | 0.58 | 0.6 | 0.27 |
| P70 S6K-T2 412 E | 54 | 7.4 | 0.5 | 0.108 |
| DN-SGK | 8 | 3.8 | 3.3 | 2.4 |
| DN-SGK 422D | 71 | 9.6 | 3.5 | 3.4 |
| FL-PKB | 28 | 19.3 | 35 | 35 |
| FL-PKB 473D | 100 | 85.4 | 178 | 162 |
| ΔPH-PKB | 0.4 | 0.25 | 0.23 | 0.25 |
| ΔPH-PKB 473D | 0.9 | 0.2 | 0.18 | 0.15 |

Relative phosphorylation of PDK1 substrates. PDK1 substrates were phosphorylated in vitro, subjected to SDS-PAGE, and radioactivity associated with the bands measured using a Phospho-Imager and known amounts of ATP as standard. The phosphorylation rate of PKB[S473D] in the presence of PtdIns (3,4,5)P3 was 2.6 mol/mol PDK1/min and was taken as the relative value of 100. Average values from a representative experiment performed in duplicates are shown.

We previously observed that in the presence of PIFtide, PDK1 is no longer able to phosphorylate S6K1 but retains its ability to activate PKBα [25] (see also FIGS. 19A & 19C & Table 3). Here we demonstrate that in the presence of PIFtide, PDK1's ability to phosphorylate and activate SGK1, ΔPH-PKBα as well as the hydrophobic motif mutants, S6K1[T412E], SGK1[T422D] and ΔPH-PKBα[S473D] is also markedly inhibited. These results demonstrate that PDK1 requires it hydrophobic PIF binding pocket in order to phosphorylate and activate S6K1, SGK1 and ΔPH-PKBα.

Role of the hydrophobic motif on phosphorylation of S6K1, SGK1 and PKB by PDK1. We next investigated the role of the hydrophobic motif of S6K1, SGK1 and PKBα in enabling PDK1 to phosphorylate these kinases. To achieve this we mutated the-conserved Phe to Ala that lies prior to the hydrophobic motif phosphorylation site on these AGC kinases (Phe411 in S6K1, Phe 421 in SGK1 and Phe 472 in PKBα) and tested the effect that this had on the phosphorylation and activation of these kinases by PDK1. This mutation was selected as the equivalent mutation in PRK2 and PKCζ prevented PDK1 from interacting and phosphorylating these mutants. The rate at which PDK1 phosphorylated S6K1[F411A], SGK1[F421A] and ΔPH-PKBα[F472A] compared to the wild type enzymes, was markedly reduced. Interestingly the basal rate at which PDK1 phosphorylated these enzymes was not inhibited by PIFtide and was comparable to the rate at which these were phosphorylated by PDK1[L155E]. In contrast, PKB[F472A] was phosphorylated at a similar rate to the wild type PKB.

We also expressed the wild type and the hydrophobic motif mutants of S6K1, SGK, PKBα and ΔPH-PKBα in 293 cells and measured the phosphorylation of these enzymes at their T-loop site before and after stimulation of cells with IGF1 using the appropriate phospho-specific antibodies. IGF1 stimulation of cells failed to induce the phosphorylation of S6K1[F411A], SGK1[F421A] and ΔPH-PKBα[F472A] at their T-loop residue, whereas the wild type kinases became phosphorylated (FIGS. 21D and 21E). S6K1[F411A] and SGK1[F421A] were also not significantly activated following IGF1 stimulation which is consistent with the lack of phosphorylation of these enzymes at their T-loop residue (data not shown). In contrast, PKBα[F472A] was phosphorylated to similar extent as wild type PKBα in response to IGF1 (FIG. 18F). PKBα[F472A] in unstimulated cells possessed ^{˜}50% of the basal activity of wild type PKBα, but its activity was not further increased following stimulation with IGF1, despite becoming phosphorylated at Thr308 (data not shown). In parallel experiments, IGF1 induced equivalent phosphorylation of wild type-PKBα at Thr308 and increased its activity over 10-fold. These results demonstrate that the hydrophobic motifs of S6K1, SGK1 and ΔPH-PKBα are required in order for these kinases to become phosphorylated by PDK1 efficiently at their T-loop motif.

Role of Lys115, Ile119 and Gln150 in the PDK1 “PIF-Pocket” on activation of S6K1, SGK1. In addition to Leu 155, there are other residues that were predicted to form part of the PIF binding pocket on PDK1, namely Lys115, Ile119 and Gln150 [17]. PDK1[K115A], PDK1[I119A], PDK1[Q150A] □ in addition to PDK1[L155A] (unpublished data) that possess ^{˜}10-fold decreased affinity for PIFtide, while retaining activity towards peptide substrates that do not interact with the PIF binding pocket indicating that they are not catalytically impaired. The rate at which PDK1[K115A], PDK1[I119A], PDK1[Q150A] and PDK1[L155A] activated S6K1[412E] was only marginally lower than wild type PDK1. In contrast the rate at which these mutants of PDK1 activated SGK1[T422D] was markedly reduced, indicating that Lys115, Ile119 and Gln150 may play a more dominant role in permitting PDK1 to phosphorylate SGK1[T422D] than S6K1[T412E]. Consistent with the reduced affinity of these mutants of PDK1 for PIFtide, low concentrations of PIFtide (2 μM) only inhibited the activation of S6K1-T2[T412E] and SGK1[T422D] by ^{˜}50% and high concentrations of PIF tide (35 μM) were required to inhibit these enzymes over 95%. Under identical conditions 2 μM PIFtide inhibited the activation of S6K1[T412E] by wild type PDK1 over ^{˜}20-fold.

Interaction of S6K1 and SGK1 with PDK1. Full length S6K1 is a very poor substrate for PDK1 compared to S6K1 lacking the C-terminal 104 amino acids in its regulatory domain [15, 27]. We therefore tested whether this could be explained by the inability of full length S6K1 to interact with PDK1. To test this hypothesis we coexpressed in 293 cells GST-PDK1 together with full length S6K1, fill length S6K1[T412E] and the C-terminal truncated forms of these mutants (S6K1-T2 and S6K1-T2[T412E]) which have Flag epitope tags. Glutathione-Sepharose “pull-downs” of GST-PDK1 from these extracts were immunoblotted for the presence of Flag epitope tagged S6K1. Although GST-PDK1 and wild type and mutant forms of S6K1 were expressed to a similar level, full length S6K1 and full length S6K1[T412E] failed to interact with GST-PDK1, whilst the S6K1-T2 and S6K1-T2[T412E] both interacted with GST-PDK1. S6K1-T2[T412E] interacted moderately better with GST-PDK1 compared to S6K1-T2. Under similar conditions we were unable to detect an interaction between GST-PDK1[L155E] and S6K1-T2 and S6K1-T2[T412E], indicating that PDK1 interacts with S6K1-T2 through the PIF binding pocket.

Wild type SGK1 is phosphorylated at a 10-fold lower rate than SGK1[T422D] (Table 3 and [9]). We therefore tested whether this could be accounted for by differences in affinity of wild type SGK1 and SGK1[T422D] for PDK1. To investigate this we coexpressed GST-SGK1 and GST-SGK1[T422D] with Myc-PDK1 in 293 cells. Glutathione-Sepharose “pull-downs” of GST-SGK1 were immunoblotted for the presence of Myc-PDK1. As shown in FIG. 20B Myc-PDK1 only interacted with SGK1[T422D] but did not interact with the wild type SGK1. As expected SGK1[T422D] failed to interact with Myc-PDK1[L155E].

Discussion

The results presented in this Example indicate that the hydrophobic motif of S6K1 and SGK1 function as a PDK1 docking site that binds to the PIF binding pocket of PDK1. This recruits PDK1 to S6K1 and SGK1 enabling PDK1 to phosphorylate these enzymes at their T-loop site. This conclusion is supported by the finding that a mutant of PDK1 in which the PIF binding pocket has been disrupted (PDK1[L155E]) can not phosphorylate S6K1 or SGK1 (FIG. 18) and mutants of S6K1 and SGK1 in which their hydrophobic motif have been disrupted can not be phosphorylated by wild type PDK1 (FIG. 19). These obsevations explain why PIFtide inhibits the phosphorylation of S6K1 and SGK1 (FIG. 20) as it interacts with the PIF binding pocket of PDK1 thus preventing it from binding to S6K1 and SGK1. Overall these results provide further evidence that AGC kinases (other than PKB) interact through their C-terminal non catalytic residues with the PIF binding pocket of PDK1. These interactions will play an important role in regulating the access to PDK1 to its substrates.

S6K1 requires phosphorylation of both the T-loop and hydrophobic motif to be activated [6] thus phosphorylation of S6K1 at its T-loop site by PDK1 alone does not significantly activate it. Full length S6K1 is a very poor substrate for PDK1 compared to a mutant of S6K1 that lacks its C-terminal 104 residues encompassing the five in vivo Ser-Pro/Thr-Pro phosphorylation sites [15, 27]. We were unable to detect an interaction between full length S6K1 or S6K1[T412E] and PDK1 whilst a full length S6K1 mutant in which the hydrophobic motif phosphorylation site (Thr412) and all five phosphorylated C-terminal residues were mutated to acidic residues, was able to bind to PDK1 but not to PDK1[L155E].

Removal of the C-terminal 104 residues of S6K1, also enabled S6K1-T2 and S6K1-T2[T412E] to interact with PDK1 but not PDK1[L155E]. S6K1-T2[T412E] is phosphorylated by PDK1 at a 5-fold higher initial rate than S6K1-T2 and consistent with previous binding studies [25] we observed that S6K1-T2[T412E] interacted with higher affinity to PDK1 than S6K1-T2. These findings suggest a model for the activation of S6K1 in which the first step would involve the phosphorylation of the C-terminal Ser-Pro/Thr-Pro by a proline directed kinase. This would not directly activate S6K1 but induce a conformational change exposing its hydrophobic motif so that it can interact with the PIF binding pocket of PDK1, enabling PDK1 to phosphorylate the T-loop residue of S6K1. This is consistent with the finding that in response to insulin stimulation of cells the C-terminal Ser-Pro/Thr-Pro of S6K1 become phosphorylated before phosphorylation of the T-loop and hydrophobic motif [6, 23]. The interaction of PDK1 with S6K1 would be enhanced further if S6K1 was phosphorylated at its hydrophobic motif. However, this may not be a prerequisite for T-loop phosphorylation as a mutant of S6K1 in which Thr412 is mutated to an Ala is still phosphorylated at its T-loop residue albeit to a lower extent that wild type S6K1 [23].

SGK1, like S6K1 requires phosphorylation of both its T-loop and hydrophobic motif to be activated in cells, but does not possess a C-terminal tail following its hydrophobic motif that becomes phosphorylated at Ser-Pro/Thr-Pro motifs. Wild type SGK1 that has not been phosphorylated at its hydrophobic motif (Thr422) is a poor substrate for PDK1 and mutation of Thr412 to an acidic residue increases over 10-fold the rate at which it becomes phosphorylated by PDK1 (Table 3 and [9]). Furthermore, when SGK1[T422D] is expressed in unstimulated 293 cells it is significantly phosphorylated at its T-loop residue (Thr256) whilst wild type SGK1 is not [8, 9]. The finding that SGK1[T422D] interacts with PDK1 (but not with PDK1[L155E]), in contrast no detectable interaction between wild type PDK1 and SGK1 was observed (FIG. 21), is consistent with the conclusion of Kobayashi & Cohen [9] that the phosphorylation of SGK1 at its hydrophobic motif plays the major role in regulating phosphorylation of SGK1 at its T-loop residue. Consistent with this, a mutant of SGK1 in which the hydrophobic motif phosphorylation site (THr422) is changed to Ala does not become phosphorylated at its T-loop phosphorylation site (THr256) following IGF1 stimulation, whilst changing T422 to Asp results in SGK1 being phosphorylated at Thr256 in unstimulated cells.

Although the activation of S6K1 and SGK1 is dependent upon PI 3-kinase in vivo, it is not clear how PtdIns(3,4,5)P₃/PtdIns(3,4)P₂ regulates this process. The phosphorylation of S6K1 and SGK1 in vitro by PDK1 is not enhanced by the presence of PtdIns(3,4,5)P₃/PtdIns(3,4)P₂ which interact with the PH domain of PDK1. This indicates that the binding of PDK1 to PtdIns(3,4,5)P₃/PtdIns(3,4)P₂ may not directly activate these enzymes. Instead of regulating the activity of PDK1, PtdIns(3,4,5)P₃/PtdIns(3,4)P₂ could instead induce activation of the kinase(s) that phosphorylate the hydrophobic motif of S6K1 and SGK1 and/or the proline directed kinase(s) that phosphorylate S6K1 at its C-terminal tail. If this mechanism operated in vivo, PDK1 activity in cells would not need to be activated by insulin or growth factors as it would not be able to phosphorylate S6K1 or SGK1 until these enzymes were phosphorylated at their hydrophobic motif/C-terminal tail.

The finding in this study that the PIF binding pocket of PDK1 is not required to enable PDK1 to activate PKB, nor is the hydrophobic motif of PKB required to allow it to become phosphorylated at its T loop supports the conclusion of previous studies indicating that binding of PDK1 and PKB to PtdIns(3,4,5)P₃/PtdIns(3,4)P₂ is likely to be the primary determinant for bringing these molecules together [14]. This is also supported by the finding that the activation of PKB, like the activation of PI 3-kinase occurs very rapidly in cells thus indicating that the activation of PKB occurs shortly after the formation of PtdIns(3,4,5)P₃/PtdIns(3,4)P₂. Instead, as the activation of S6K1 (and SGK1) occurs much more slowly than PKB; this indicates that there is a substantial delay between activation of PI 3-kinase and activation of S6K1 and SGK1. This delay could be accounted for by the time it takes for PtdIns(3,4,5)P₃/PtdIns(3,4)P₂ to activate the S6K1-C-terminal Ser-Pro/Thr-Pro kinase(s) and the S6K1/SGK1 hydrophobic motif kinase(s) which are likely be the rate limiting step in the activation of these kinases in cells. There is no evidence that phosphorylation of PKBα at Ser473 promotes phosphorylation of Thr308, as mutation of Ser473 to either Ala or Asp had no effect on insulin/IGF1 induced phosphorylation of PKBα at Thr308 [24]. It can not be ruled out that PDK1 could interact with PKBα through a domain other than the PIF binding pocket and the binding of PKB and/or PDK1 to PtdIns(3,4,5)P₃/PtdIns(3,4)P₂ could expose this binding pocket(s). Indeed, there is evidence that the binding of PKB to PtdIns(3,4,5)P₃/PtdIns(3,4)P₂ could induce a conformational change leading to the exposure of the Thr308 and perhaps Ser473 phosphorylation sites [14]. VanObberghen and colleagues have also concluded that the PH domain of PDK1 may inhibit it from phosphorylating PKBα [28]. This is based on the finding that a PDK1 mutant lacking its PH domain or possessing a PH domain that can not interact with PtdIns(3,4,5)P₃ was far more effective at activating ΔPH-PKBα in a cotransfection experiment than the wild type PDK1. Although it should be noted that this observed in vivo effect is likely to be more complicated, as the rate at which ΔPH-PDK1 and wild type PDK1 phosphorylate ΔPH-PKBα is similar in vitro [22,29].

ΔPH-PKBα when expressed in 293 cells is phosphorylated at Thr308 and Ser473 in response to insulin and this is prevented by inhibitors of PI 3-kinase [30, 31]. This observation was originally interpreted as evidence that PDK1 and the enzyme which phosphorylates Ser473 were activated in vivo by PtdIns(3,4,5)P₃/PtdIns(3,4)P₂. However, in this study, we demonstrate that ΔPH-PKBα is not phosphorylated by PDK1 by the same mechanism as wild type PKBα, as ΔPH-PKBα is not phosphorylated by PDK1[L155E] and disruption of the hydrophobic motif of ΔPH-PKBα largely prevented its phosphorylation by PDK1 (FIGS. 18 to 19). Thus the mechanism by which PDK1 phosphorylates ΔPH-PKBα is more like S6K1 and SGK1 than PKBα. As mutation of Ser473 in ΔPH-PKBα to Asp increases the rate at which it is phosphorylated by PDK1 (Table 3), it is possible that when ΔPH-PKBα is expressed in cells PtdIns(3,4,5)P₃/PtdIns(3,4)P₂ does not activate PDK1 but instead induces phosphorylation of Ser473 through the same hydrophobic motif kinase(s) that phosphorylate S6K1 and SGK1, which subsequently converts ΔPH-PKBα into a PDK1 substrate.

Although ΔPH-PKBα is phosphorylated by PDK1 at the same rate in the presence or absence of PtdIns(3,4,5)P₃ it should be emphasised that ΔPH-PKBα is phosphorylated by PDK1 at ^{˜}50-fold lower rate than wild type PKBα in the presence of PtdIns(3,4,5)P₃. Furthermore, ΔPH-PKBα is phosphorylated in vitro at a 10-100-fold lower rate than SGK1 and S6K1 by PDK1 (Table 3). This might be explained if the C-terminal region of ΔPH-PKBα surrounding the hydrophobic motif, interacted with significantly lower affinity with PDK1 than the equivalent region of S6K1 and SGK1. It is possible that this region of S6K1 and SGK1, has evolved to enable these enzymes to bind PDK1. However, the equivalent residues in PKB may not have evolved not to interact with the PIF binding pocket of PDK1, enabling PKB to be regulated by a distinct mechanism.

When PKCζ and PRK2 are overexpressed in unstimulated 293 cells unlike S6K1, SGK1 and PKB they are phosphorylated at their T-loop residue to a high stoichiometry and when these kinases are isolated from cells can not be phosphorylated further by PDK1 in vitro. We also demonstrated that PRK2 and PKCζ and could directly interact through their hydrophobic motif to the PIF binding pocket of PDK1 [18]. These observations imply that in contrast to S6K1, SGK1 and PKB when PKCζ or PRK2 is overexpressed in unstimulated cells, their hydrophobic motif is able to interact directly with endogenous PDK1 resulting in their T-loop residue becoming phosphorylated. However, it is likely that phosphorylation of the T-loop residue of endogenously expressed PKCζ and PRK2 will be under some regulation in cells. Parker and colleagues have presented evidence that the interaction of PRK2 with the small GTP binding protein Rho complexed to GTP, may promote the interaction of PRK2 with PDK1 and enhance the phosphorylation of its T-loop residue [32]. In the case of PKCζ it is not clear how phosphorylation of its T-loop is regulated. Although there are several reports indicating that PKCζ is activated by insulin and growth factors in cells and that these agonists induce phosphorylation of PKCζ at its T-loop residue, we have thus far not been able to demonstrate any further activation of either endogenous or transfected PKCζ in 293 cells. In unstimulated mouse embryonic stem cells endogenous PKCζ is significantly phosphorylated at its T-loop residue and this is not further increased by stimulation with IGF1 or any other agonist that we have tried [33]. In mouse embryonic stem cells lacking PDK1, PKCζ is not phosphorylated at its T-loop residue, providing genetic evidence that PDK1 mediates phosphorylation of PKCζ in vivo [33]. Recent work demonstrates that PKCζ in cells is complexed to other proteins termed hPar3 and hPar6 and that hPar6 in this complex is capable of interacting with the small GTP binding proteins Rac and CDC42 (reviewed in [34]). The evidence indicates that these proteins will play key roles in regulating the activity of PKCζ. However, it has not yet been investigated whether hPar3/hPar6/CDC42/Rac1 could also function by regulating the phosphorylation PKCζ by PDK1. This complex could operate by controlling the access of the hydrophobic motif of PKCζ to enable interaction of PDK1 in response to a specific signal. For example the binding of Rac/CDC42 to this complex may enable PDK1 to interact and phosphorylate PKCζ.

The model we propose in FIG. 22 demonstrates the key step in the phosphorylation of PDK1 substrates thus far been identified other than PKB are regulated by the direct interaction of their hydrophobic motif with the PIF binding pocket of PDK1. Instead PKB and PDK1 are brought together mainly by their mutual interaction with PtdIns(3,4,5)P₃. Although PRK2 and PKCζ are can interact directly with PDK1 when overexpressed in 293 cells, the interaction of S6K1 and SGK1 is regulated by the phosphorylation of these enzymes at their C-terminal residue(s). This model could account for the differences in the time course of activation of S6K1, SGK1 and PKB in IGF1/growth factor stimulated cells. These findings indicate that drugs directed towards a specific non catalytic site on a protein kinase could inhibit the phosphorylation of a group of substrates without affecting the phosphorylation of another. Thus compounds that interact with the PIF-binding pocket on PDK1 could affect the activation of S6K1 and SGK1 but not PKB and will be more specific than an ATP competitive PDK1 inhibitor which will inhibit the phosphorylation of all downstream targets. Thus such drugs are likely to have less side effects.

References

1 Leevers, S. J., Vanhaesebroeck, B. and Waterfield, M. D. (1999) Curr Opin Cell Biol 11, 219-225

2 Peterson, R. T. and Schreiber, S. L. (1999) Curr Biol 9, R521-R524

3 Belham, C., Wu, S. and Avruch, J. (1999) Curr Biol 9, R93-96

4 Alessi, D. R. and Cohen, P. (1998) Curr Opin Genet Dev 8, 55-62

5 Coffer, P. J., Jin, J. and Woodgett, J. R. (1998) Biochem J 335, 1-13

6 Dufner, A. and Thomas, G. (1999) Exp Cell Res 253, 100-109

7 Avruch, J. (1998) Mol Cell Biochem 182, 31-48

8 Park, J., Leong, M. L., Buse, P., Maiyar, A. C., Firestone, G. L. and Hemmings, B. A. (1999) Embo J 18, 3024-3033

9 Kobayashi, T. and Cohen, P. (1999) Biochem J 339, 319-328

10 Kobayashi, T., Deak, M., Morrice, N. and Cohen, P. (1999) Biochem J 344, 189-197

11 Kohn, A. D., Kovacina, K. S. and Roth, R. A. (1995) Embo J 14, 4288-4295

12 Cross, D. A., Alessi, D. R., Cohen, P., Andjelkovich, M. and Hemmings, B. A. (1995) Nature 378, 785-789

13 Burgering, B. M. and Coffer, P. J. (1995) Nature 376, 599-602

14 Vanhaesebroeck, B. and Alessi, D. R. (2000) Biochem J 346, 561-576

15 Alessi, D. R., Kozlowski, M. T., Weng, Q. P., Morrice, N. and Avruch, J. (1998) Curr Biol 8, 69-81

16 Balendran, A., Casamayor, A., Deak, M., Paterson, A., Gaffney, P., Currie, R., Downes, C. P. and Alessi, D. R. (1999) Curr Biol 9, 393-404

17 Biondi, R. M., Cheung, P. C., Casamayor, A., Deak, M., Currie, R. A. and Alessi, D. R. (2000) Embo J 19, 979-988

18 Balendran, A., Biondi, R. M., Cheung, P. C., Casamayor, A., Deak, M. and Alessi, D. R. (2000) J Biol Chem 275, 20806-20813

19 Frodin, M., Jensen, C. J., Merienne, K. and Gammeltoft, S. (2000) Embo J 19, 2924-2934

20 Gaffney, P. R. J. and Reese, C. B. (1997) Bioorg.Med.Chem.Lett. 7, 3171-3176

21 Alessi, D. R., James, S. R., Downes, C. P., Holmes, A. B., Gaffney, P. R., Reese, C. B. and Cohen, P. (1997) Curr Biol 7, 261-269

22 Alessi, D. R., Deak, M., Casamayor, A., Caudwell, F. B., Morrice, N., Norman, D. G., Gaffney, P., Reese, C. B., MacDougall, C. N., Harbison, D., Ashworth, A. and Bownes, M. (1997) Curr Biol 7, 776-789

23 Weng, Q. P., Kozlowski, M., Belham, C., Zhang, A., Comb, M. J. and Avruch, J. (1998) J Biol Chem 273, 16621-16629

24 Alessi, D. R., Andjelkovic, M., Caudwell, B., Cron, P., Morrice, N., Cohen, P. and Hemmings, B. A. (1996) Embo J 15, 6541-6551

25 Balendran, A., Currie, R. A., Armstrong, C. G., Avruch, J. and Alessi, D. R. (1999) J Biol Chem 274, 37400-37406

26 Alessi, D. R., Cohen, P., Ashworth, A., Cowley, S., Leevers, S. J. and Marshall, C. J. (1995) Methods Enzymol 255, 279-290

27 Pullen, N., Dennis, P. B., Andjelkovic, M., Dufner, A., Kozma, S. C., Hemmings, B. A. and Thomas, G. (1998) Science 279, 707-710

8 Filippa, N., Sable, C. L., Hemmings, B. A. and Van Obberghen, E. (2000) Mol Cell Biol 20, 5712-5721

29 Currie, R. A., Walker, K. S., Gray, A., Deak, M., Casamayor, A., Downes, C. P., Cohen, P., Alessi, D. R. and Lucocq, J. (1999) Biochem J 337, 575-583

30 Andjelkovic, M., Alessi, D. R., Meier, R., Fernandez, A., Lamb, N. J., Frech, M., Cron, P., Cohen, P., Lucocq, J. M. and Hemmings, B. A. (1997) J Biol Chem 272, 31515-31524

31 Kohn, A. D., Takeuchi, F. and Roth, R. A. (1996) J Biol Chem 271, 21920-21926

32 Flynn, P., Mellor, H., Casamassima, A. and Parker, P. J. (2000) J Biol Chem 275, 11064-11070

33 Balendran, A., Hare, G. R., Kieloch, A., Williams, M. R. and Alessi, D. R. (2000) FEBS Lett 484, 217-223-34 Brazil, D. P. and Hemmings, B. A. (2000) Curr Biol 10, R592-594

1. A method of identifying a compound that modulates the protein kinase activity of a protein kinase having a hydrophobic pocket in the position equivalent to the hydrophobic pocket of mouse Protein Kinase A (PKA) that is defined by residues including Lys76, Leu116, Val80 and/or Lys111 of full-length mouse PKA, wherein the ability of the compound to inhibit, promote or mimic the interaction of the said hydrophobic pocket-containing protein kinase with an interacting polypeptide is measured and a compound that inhibits, promotes or mimics the said interaction is selected, wherein the interacting polypeptide interacts with the hydrophobic pocket of the protein kinase and/or comprises the amino acid sequence Phe/Tyr-Xaa-Xaa-Phe/Tyr.

2. The method of claim 1 wherein the polypeptide comprises the amino acid sequence Phe/Tyr-Xaa-Xaa-Phe/Tyr-Zaa-Phe/Tyr, wherein Zaa represents a negatively charged amino acid residue.

3. The method of claim 1 wherein the polypeptide comprises the amino acid sequence Phe-Xaa-Xaa-Phe.

4. The method of claim 1, 2 or 3 wherein the protein kinase is PDK1.

5. The method of claim 1, 2 or 3 wherein the protein kinase is SGK, PKB, PKA, p70 S6 kinase, p90 RSK, PKCα, PKCδ, PKCζ; or PRK2.

6. The method of any of the previous claims wherein the interaction is an interaction of the hydrophobic pocket of the said protein kinase with the polypeptide comprising the amino acid sequence Phe/Tyr-Xaa-Xaa-Phe/Tyr.

7. The method of any of the previous claims wherein the interacting polypeptide is part of the same polypeptide chain as the protein kinase.

8. The method of claim 7 wherein the interaction is an intramolecular interaction.

9. The method of any of the preceding claims wherein the ability of the compound to inhibit, promote or mimic the interaction of the protein kinase with the interacting polypeptide is measured using surface plasmon resonance.

10. A method of identifying a compound that modulates the protein kinase activity of a hydrophobic pocket-containing protein kinase as defined in claim 1, wherein the effect of the said compound on the rate or degree of phosphorylation of a substrate polypeptide of the said hydrophobic pocket-containing protein kinase by the said hydrophobic pocket-containing protein kinase in the presence of an interacting polypeptide is determined, and a compound that modulates the said rate or degree of phosphorylation is selected, wherein the interacting polypeptide interacts with the hydrophobic pocket of the protein kinase and/or comprises the amino acid sequence Phe/Tyr-Xaa-Xaa-Phe/Tyr and is comprised in a separate polypeptide chain to the hydrophobic pocket-containing protein kinase, and wherein the substrate polypeptide has fewer than 400 amino acids.

11. The method of claim 10 wherein the substrate polypeptide comprises a portion that is the interacting polypeptide.

12. The method of claim 11 wherein the protein kinase is PDK1 and the substrate polypeptide comprises or consists of the sequence KTFCGTPEYLAPEVRREPRILSEEEQEMFRDFDYIADWC.

13. The method of claim 10 wherein the substrate portion and the interacting portion are on separate polypeptide chains.

14. The method of claim 13 wherein the hydrophobic pocket-containing protein kinase is PDK1, the substrate polypeptide comprises or consists of the sequence KTFCGTPEYLAPEV, and the interacting polypeptide comprises or consists of the sequence EPRILSEEEQEMFRDFDYIADWC.

15. A method of identifying a compound that modulates the protein kinase activity of a hydrophobic pocket-containing protein kinase as defined in claim 1, wherein the effect of the said compound on the rate or degree of phosphorylation of a substrate polypeptide of the said hydrophobic pocket-containing protein kinase by the said hydrophobic pocket-containing protein kinase is determined, and a compound that modulates the said rate or degree of phosphorylation is selected, wherein the effect of the compound is determined in the absence of an interacting polypeptide, wherein an interacting polypeptide is one which interacts with the hydrophobic pocket of the protein kinase and/or comprises the amino acid sequence Phe/Tyr-Xaa-Xaa-Phe/Tyr, and wherein the substrate polypeptide has fewer than 400 amino acids.

16. The method of claim 15 wherein hydrophobic pocket-containing protein kinase is PDK1 and the substrate polypeptide consists of or comprises the amino acid sequence KTFCGTPEYLAPEV or KTFCGTPEYLAPEVRR.

17. A method of selecting or designing a compound that modulates the activity of a hydrophobic pocket-containing protein kinase as defined in any of the preceding claims, the method comprising the step of using molecular modelling means to select or design a compound that is predicted to interact with the said hydrophobic pocket-containing protein kinase, wherein a three-dimensional structure of a compound is compared with a three-dimensional structure of the said hydrophobic pocket and/or with a three-dimensional structure of an interacting polypeptide as defined in any of the preceding claims, and a compound that is predicted to interact with the said hydrophobic pocket is selected.

18. A compound capable of modulating the protein kinase activity of a hydrophobic pocket-containing protein kinase as defined in claim 1, wherein the compound inhibits the interaction of the said protein kinase with an interacting polypeptide, wherein the interacting polypeptide interacts with the hydrophobic pocket of the said protein kinase and/or comprises the amino acid sequence Phe/Tyr-Xaa-Xaa-Phe/Tyr, wherein the compound does not comprise a polypeptide having the amino acid sequence Phe/Tyr-Xaa-Xaa-Phe/Tyr-Zaa-Phe/Tyr and is not PKA.

19. A compound capable of modulating the protein kinase activity of a hydrophobic pocket-containing protein kinase as defined in claim 1, wherein the compound modulates the rate or degree of phosphorylation of a substrate polypeptide of the said hydrophobic pocket-containing protein kinase by the said hydrophobic pocket-containing protein kinase in the absence of an interacting polypeptide, wherein an interacting polypeptide interacts with the hydrophobic pocket of the protein kinase and/or comprises the amino acid sequence Phe/Tyr-Xaa-Xaa-Phe/Tyr, and wherein the substrate polypeptide has fewer than 400 amino acids.

20. A compound that modulates the protein kinase activity of a hydrophobic pocket-containing protein kinase as defined in claim 1, wherein the compound modulates the rate or degree of phosphorylation of a substrate polypeptide of the said hydrophobic pocket-containing protein kinase by the said hydrophobic pocket-containing protein kinase in the presence of an interacting polypeptide, wherein the interacting polypeptide interacts with the hydrophobic pocket of the protein kinase and/or comprises the amino acid sequence Phe/Tyr-Xaa-Xaa-Phe/Tyr and is comprised in a separate polypeptide chain to the hydrophobic pocket-containing protein kinase, and wherein the substrate polypeptide has fewer than 400 amino acids.

21. A compound identifiable by the method of any one of claims 1 to 17, provided that the compound is not a polypeptide having the amino acid sequence Phe/Tyr-Xaa-Xaa-Phe/Tyr-Zaa-Phe/Tyr and is not full length PKA.

22. A mutated protein kinase, wherein the protein kinase before mutation has a hydrophobic pocket in the position equivalent to the hydrophobic pocket of mouse Protein Kinase A (PKA) that is defined by residues including Lys76, Leu116, Val80 and/or Lys111 of full-length mouse PKA, and wherein one or more residues defining the hydrophobic pocket of the protein kinase is mutated.

23. The mutated protein kinase of claim 22 wherein the protein kinase before mutation is PDK1, SGK, p70 S6 kinase or PKB.

24. The mutated protein kinase of claim 23 wherein the mutated residue(s) are the residues equivalent to residue Lys76, Val80, Lys111 and/or Leu116 of full length mouse PKA.

25. The mutated protein kinase of claim 24 wherein the residue at the position equivalent to residue Lys76 of fill length mouse PKA is mutated to an Ala and/or the residue at the position equivalent to Leu116 of full length mouse PKA is mutated to a Ser, Asp or Glu.

26. A preparation comprising a hydrophobic pocket-containing protein kinase as defined in claim 1, and a second, interacting compound, wherein the interacting compound interacts with the hydrophobic pocket of the protein kinase and/or comprises the amino acid sequence Phe/Tyr-Xaa-Xaa-Phe/Tyr, wherein the said preparation further comprises a substrate polypeptide as defined in any of claims 10 to 16 and does not comprise all of the components found in a cell in which said protein kinase or compound is naturally found.

27. A preparation comprising a hydrophobic pocket-containing protein kinase as defined in claim 1, and a second, interacting compound, wherein the interacting compound interacts with the hydrophobic pocket of the protein kinase, wherein the said preparation does not comprise all of the components found in a cell in which said protein kinase or compound is naturally found, and wherein when the protein kinase is PDK1, the interacting compound is not a polypeptide comprising the amino acid sequence Phe/Tyr-Xaa-Xaa-Phe/Tyr-Zaa-Phe/Tyr.

28. A method of phosphorylating a substrate polypeptide for a hydrophobic pocket-containing protein kinase as defined in claim 1, wherein a preparation according to claim 27 is used.

29. A method of phosphorylating p70 S6 kinase on the residue equivalent to Thr412 of full length human p70 S6 kinase wherein the said p70 S6 kinase is exposed to recombinant PDK1.

30. A method of identifying a compound that modulates the activation and/or phosphorylation of p70 S6 kinase on the residue equivalent to Thr412 of full length human p70 S6 kinase by PDK1 wherein the activation and/or phosphorylation of p70 S6 kinase on the residue equivalent to Thr412 of full length human p70 S6 kinase by PDK1 is measured in the presence of more than one concentration of the compound.

31. A compound identified or identifiable by the method of claim 30.

32. The use of an interacting polypeptide as defined in claim 1 or a compound as defined in any of claims 18 to 21 in a method of stabilising a hydrophobic pocket-containing protein kinase as defined in claim 1, wherein the protein kinase is exposed to the compound or polypeptide.

33. A method of modulating in a cell the protein kinase activity of a hydrophobic pocket-containing protein kinase as defined in claim 1, wherein a recombinant interacting polypeptide is expressed in the cell, wherein the interacting polypeptide interacts with the hydrophobic pocket of the protein kinase and/or has the amino acid sequence Phe/Tyr-Xaa-Xaa-Phe/Tyr.

34. A polypeptide comprising the amino acid sequence Phe/Tyr-Xaa-Xaa-Phe/Tyr, wherein said polypeptide does not comprise the amino acid sequence Phe/Tyr-Xaa-Xaa-Phe/Tyr-Zaa-Phe/Tyr or Phe/Tyr-Xaa-Xaa-Phe/Tyr-Ser/Thr-Phe/Tyr and is not full-length PKA.

35. A polypeptide according to claim 34 comprising or consisting essentially of the C-terminal 223 amino acids of full length PKA.

36. A fusion polypeptide of a polypeptide according to claim 34 or 35, wherein the fusion polypeptide is not full length PKA.

37. A polynucleotide encoding a polypeptide according to any one of claims 34 to 36 or a mutated protein kinase according to any of claims 22 to 25.

38. A recombinant polynucleotide suitable for expressing a polypeptide according to any one of claims 34 to 36 or a mutated protein kinase according to any of claims 22 to 25.

39. A host cell comprising a polynucleotide according to claim 37 or 38.

40. A method of making a polypeptide according to any one of claims 34 to 36 or a mutated protein kinase according to any of claims 22 to 25, the method comprising culturing a host cell according to claim 39 which expresses said polypeptide or mutated protein kinase and isolating said polypeptide.

41. A polypeptide obtainable by the method of claim 40.

42. A cell containing a recombinant nucleic acid suitable for expressing a hydrophobic pocket-containing protein kinase as defined in claim 1, and a recombinant nucleic acid suitable for expressing a second polypeptide comprising the amino acid sequence Phe/Tyr-Xaa-Xaa-Phe/Tyr, wherein when the said protein kinase is PDK1, the said second polypeptide is not EDVKKHPFFRLIDWSALMDKKVKPPFIPTIRGREDVSNFDDEFTSEAPILTPPREPRILSEEEQEMFRDFDYIADWC (PIF), and does not comprise the amino acid sequence Phe/Tyr-Xaa-Xaa-Phe/Tyr-Ser/Thr-Phe/Tyr.

43. A method of making a preparation comprising a hydrophobic pocket-containing protein kinase as defined in claim 1, and a second, interacting polypeptide, wherein the interacting polypeptide interacts with the hydrophobic pocket of the protein kinase, wherein the said preparation does not comprise all of the components found in a cell in which said protein kinase or compound is naturally found, and wherein when the protein kinase is PDK1, the interacting polypeptide is not a polypeptide comprising the amino acid sequence Phe/Tyr-Xaa-Xaa-Phe/Tyr-Zaa-Phe/Tyr, wherein the said protein kinase and the said interacting polypeptide are co-expressed in a cell according to claim 42.

44. A kit of parts useful in carrying out a method according to any one of claims 1 to 16, comprising a hydrophobic pocket-containing protein kinase as defined in claim 1 and a separate interacting polypeptide wherein the interacting polypeptide interacts with the hydrophobic pocket of the said protein kinase and/or comprises the amino acid sequence Phe/Tyr-Xaa-Xaa-Phe/Tyr and does not comprise the amino acid sequence Phe/Tyr-Xaa-Xaa-Phe/Tyr-Zaa-Phe/Tyr or Phe/Tyr-Xaa-Xaa-Phe/Tyr-Ser/Thr-Phe/Tyr.

45. A kit of parts useful in carrying out a method according to any one of claims 10 to 16, comprising a hydrophobic pocket-containing protein kinase as defined in claim 1 and a substrate polypeptide as defined in any one of claims 10 to 16 and optionally a separate interacting polypeptide wherein the interacting polypeptide interacts with the hydrophobic pocket of the said protein kinase and/or comprises the amino acid sequence Phe/Tyr-Xaa-Xaa-Phe/Tyr.

46. A compound according to claim 18 to 21 or 31, or polypeptide according to any of claims 34 to 36 or polynucleotide according to claim 37 or 38 for use in medicine.

47. The use of a compound or polypeptide or polynucleotide as defined in claim 46 in the manufacture of a medicament for the treatment of a patient in need of modulation of signalling by a hydrophobic pocket-containing protein kinase as defined in claim 1.

48. The use of claim 47 wherein the patient has cancer or diabetes or is in need of inhibition of apoptosis, for example a patient suffering from tissue injury or ischaemic injury, including stroke.

49. A polypeptide comprising non-overlapping interacting and substrate portions, wherein the interacting portion comprises the amino acid sequence Phe/Tyr-Xaa-Xaa-Phe/Tyr and the substrate portion comprises a consensus sequence for phosphorylation by a hydrophobic pocket-containing protein kinase as defined in claim 1, wherein the amino acid sequence Phe/Tyr-Xaa-Xaa-Phe/Tyr and the said consensus sequence for phosphorylation are separated by between about 5 and 100 amino acids.

50. An interacting polypeptide which interacts with the hydrophobic pocket of a hydrophobic pocket-containing protein kinase as defined in claim 1 and/or comprises the amino acid sequence Phe/Tyr-Xaa-Xaa-Phe/Tyr, immobilised on a surface of an article suitable for use as a test surface in a surface plasmon resonance method, wherein the interacting polypeptide is not PIF or PIFtide.

51. A method of identifying a compound that modulates the protein kinase activity of a protein kinase having a hydrophobic pocket in the position equivalent to the hydrophobic pocket of mouse Protein Kinase A (PKA) that is defined by residues including Lys76, Leu116, Val80 and/or Lys111 of full-length mouse PKA comprising the steps of (1) determining the effect of a test compound on the protein kinase activity of the said protein kinase, and/or a mutant thereof, and (2) selecting a compound capable of modulating the protein kinase activity of the said protein kinase to different extents towards (i) a substrate that binds to the said hydrophobic pocket of the said protein kinase (hydrophobic pocket-dependent substrate) and (ii) a substrate (such as PKB) that does not bind, or binds to a lesser extent than the first said substrate (hydrophobic pocket-independent substrate), to the said hydrophobic pocket of the said protein kinase.

52. The method of claim 51 wherein a compound that inhibits the protein kinase activity of the said protein kinase to a greater extent towards the hydrophobic pocket-dependent substrate than towards the hydrophobic pocket-independent substrate is selected.

53. The method of claim 51 or 52 wherein the protein kinase is PDK1.

54. The method of claim 53 wherein the hydrophobic pocket-dependent substrate is SGK, PRK2, S6K1 or PKCζ.

55. The method of claim 53 or 54 wherein the hydrophobic pocket-independent substrate is PKB.

56. A method of identifying a compound that modulates the protein kinase activity of a protein kinase having a hydrophobic pocket in the position equivalent to the hydrophobic pocket of mouse Protein Kinase A (PKA) that is defined by residues including Lys76, Leu116, Val80 and/or Lys111 of full-length mouse PKA (for example PDK1), comprising the step of determining the effect of the compound on the protein kinase activity of, or ability of the compound to bind to (1) the said protein kinase mutated at a residue defining at least part of the said hydrophobic pocket of the protein kinase, for example the residue equivalent to lysine 76 of full-length mouse PKA.

57. A method according to claim 56 further comprising determining the effect of the compound on the protein kinase activity of, or ability of the compound to bind to, the protein kinase which is not mutated at the said residue defining at least part of the said hydrophobic pocket of PDK1.

58. The method of any one of claims 51 to 57 wherein the effect of the compound on the rate or degree of phosphorylation of a hydrophobic pocket-dependent substrate is determined.

59. The method of any one of claims 51 to 57 wherein a compound is selected that decreases the protein kinase activity of the protein kinase towards a hydrophobic pocket-dependent substrate and does not affect or increases the protein kinase activity of the protein kinase towards a hydrophobic pocket-independent substrate.

60. A kit of parts useful in carrying out a method according to any one of claims 51 to 59, comprising (1) a mutated protein kinase as defined in claim 56 and/or the protein kinase which is not a mutated said protein kinase as defined in claim 56 and (2) a hydrophobic pocket-dependent substrate and a hydrophobic pocket-independent substrate of the said protein kinase.

61. The use of a compound capable of inhibiting to a different extents the rate or degree of phosphorylation by a protein kinase having a hydrophobic pocket in the position equivalent to the hydrophobic pocket of mouse Protein Kinase A (PKA) that is defined by residues including Lys76, Leu116, Val80 and/or Lys111 of full-length mouse PKA (for example PDK1), of (1) a hydrophobic pocket-dependent substrate and (2) a hydrophobic pocket-independent substrate of the protein kinase, in the manufacture of a medicament for the treatment of a patient in need of inhibition to different extents of (1) phosphorylation of a hydrophobic pocket-dependent substrate of the said protein kinase and (2) phosphorylation of a hydrophobic pocket-dependent substrate of the said protein kinase.

62. The use according to claim 61 wherein the compound or composition inhibits to a greater degree the rate or degree of phosphorylation by the protein kinase of (1) a hydrophobic pocket-dependent substrate of the protein kinase than (2) a hydrophobic pocket-independent substrate of the protein kinase.

63. The use of claim 61 or 62 wherein the patient has diabetes or cancer.


SEQUENCE LISTING (68 sequences)

SEQ ID NO: 1 - protein, 4 aa, Artificial Sequence
  VARIANT (1): Tyr or Phe
XXXX

SEQ ID NO: 2 - protein, 6 aa, Artificial Sequence
  VARIANT (1): Tyr or Phe
XXXXXX

SEQ ID NO: 3 - protein, 39 aa, Artificial Sequence
  Description of Artificial Sequence Polypeptide
KTFCGTPEYLAPEVRREPRILSEEEQEMFRDFDYIADWC

SEQ ID NO: 4 - protein, 14 aa, Artificial Sequence
  Description of Artificial Sequence Polypeptide
KTFCGTPEYLAPEV

SEQ ID NO: 5 - protein, 23 aa, Artificial Sequence
  Description of Artificial Sequence Polypeptide
EPRILSEEEQEMFRDFDYIADWC

SEQ ID NO: 6 - protein, 16 aa, Artificial Sequence
  Description of Artificial Sequence Polypeptide
KTFCGTPEYLAPEVRR

SEQ ID NO: 7 - protein, 6 aa, Artificial Sequence
  VARIANT (1): Tyr or Phe
XXXXXX

SEQ ID NO: 8 - protein, 77 aa, Artificial Sequence
  Description of Artificial Sequence Polypeptide
EDVKKHPFFRLIDWSALMDKKVKPPFIPTIRGREDVSNFDDEFTSEAPILTPPREPRILSEEEQEMFRDFDYIADWC

SEQ ID NO: 9 - protein, 8 aa, Artificial Sequence
  VARIANT (2) or (3): Variable amino acid
FXXFGGGG

SEQ ID NO: 10 - protein, 5 aa, Artificial Sequence
  Description of Artificial Sequence Consensus sequence
FEGFA

SEQ ID NO: 11 - protein, 5 aa, Artificial Sequence
  Description of Artificial Sequence Consensus sequence
FAGFS

SEQ ID NO: 12 - protein, 6 aa, Artificial Sequence
  VARIANT (1): Tyr or Phe
XXXXXX

SEQ ID NO: 13 - protein, 6 aa, Artificial Sequence
  VARIANT (1): Tyr or Phe
XXXXXX

SEQ ID NO: 14 - protein, 11 aa, Artificial Sequence
  Description of Artificial Sequence Consensus sequence
GRPRTSSFAEG

SEQ ID NO: 15 - protein, 7 aa, Artificial Sequence
  Description of Artificial Sequence Peptide
RPRAATF

SEQ ID NO: 16 - protein, 7 aa, Artificial Sequence
  Description of Artificial Sequence Peptide
LRRASLG

SEQ ID NO: 17 - protein, 11 aa, Artificial Sequence
  Description of Artificial Sequence Peptide
EILSRRPSYRK

SEQ ID NO: 18 - protein, 6 aa, Artificial Sequence
  VARIANT (1): Tyr or Phe
XXXXXX

SEQ ID NO: 19 - protein, 8 aa, Artificial Sequence
  VARIANT (1): Ser or Thr
XFCGTXEL

SEQ ID NO: 20 - protein, 5 aa, Artificial Sequence
  VARIANT (3): Any small residue
RRXSX

SEQ ID NO: 21 - protein, 6 aa, Artificial Sequence
  VARIANT (6): Thr or Ser
RXRXXX

SEQ ID NO: 22 - protein, 24 aa, Artificial Sequence
  Description of Artificial Sequence Peptide
REPRILSEEEQEMFRDFDYIADWC

SEQ ID NO: 23 - protein, 24 aa, Artificial Sequence
  Description of Artificial Sequence Peptide
REPRILSEEEQEMARDFDYIADWC

SEQ ID NO: 24 - protein, 24 aa, Artificial Sequence
  Description of Artificial Sequence Peptide
REPRILSEEEQEMFGDFDYIADWC

SEQ ID NO: 25 - protein, 53 aa, Artificial Sequence
  Description of Artificial Sequence Polypeptide
EDVKKHPFFRLIDWSALMDKKVKPPFIPTIRGREDVSNFDDEFTSEAPILTPP

SEQ ID NO: 26 - protein, 5 aa, Artificial Sequence
  VARIANT (3): Variable amino acid
RRXSV

SEQ ID NO: 27 - protein, 16 aa, Artificial Sequence
  Description of Artificial Sequence Polypeptide
KTFCGTPEYLAPEVRR

SEQ ID NO: 28 - protein, 18 aa, Artificial Sequence
  Description of Artificial Sequence Peptide
SESANQVFLGFTYVAPSV

SEQ ID NO: 29 - DNA, 52 bp, Artificial Sequence
  Description of Artificial Sequence PCR primer
aggatccaccatgcaccatcaccatcaccatatgaggcgagcaaggaggcgg

SEQ ID NO: 30 - DNA, 33 bp, Artificial Sequence
  Description of Artificial SequencePCR primer
gcggccgctcaactttcaagtacagatggagcc

SEQ ID NO: 31 - protein, 24 aa, Artificial Sequence
  Description of Artificial Sequence Peptide
REPRILSEEEQEMFRDFAYLADWC

SEQ ID NO: 32 - protein, 26 aa, Artificial Sequence
  Description of Artificial Sequence Peptide
YRRAAVPPSPSLSRHSSPHQAEDEEE

SEQ ID NO: 33 - protein, 25 aa, Artificial Sequence
  Description of Artificial Sequence Peptide
KKVKPPFIPTIRGREDVSNFDDEFT

SEQ ID NO: 34 - protein, 39 aa, Artificial Sequence
  Description of Artificial Sequence Peptide
KTFCGTPEYLAPEVRREPRILSEEEQEMFRDFDYLADWC

SEQ ID NO: 35 - protein, 13 aa, Artificial Sequence
  Description of Artificial Sequence Peptide
KDGATMKTFCGTP

SEQ ID NO: 36 - protein, 15 aa, Artificial Sequence
  Description of Artificial Sequence Peptide
HDGTVTHTFCGTIEY

SEQ ID NO: 37 - protein, 12 aa, Artificial Sequence
  VARIANT (6) or (7): Variable amino acid
TFCGTXXYXAPD

SEQ ID NO: 38 - protein, 12 aa, Artificial Sequence
  Description of Artificial Sequence Consensus
TFCGTPEYLAPE

SEQ ID NO: 39 - protein, 12 aa, Homo sapiens
TFCGTPDYIAPE

SEQ ID NO: 40 - protein, 12 aa, Homo sapiens
TFCGTPNYIAPE

SEQ ID NO: 41 - protein, 12 aa, Homo sapiens
TFCGTPEFLAPE

SEQ ID NO: 42 - protein, 12 aa, Homo sapiens
TFCGTIEYMAPE

SEQ ID NO: 43 - protein, 12 aa, Homo sapiens
SFCGTVEYMAPE

SEQ ID NO: 44 - protein, 12 aa, Homo sapiens
SFCGTIEYMAPE

SEQ ID NO: 45 - protein, 6 aa, Artificial Sequence
  VARIANT (2) or (3): Variable amino acid
FXXFSY

SEQ ID NO: 46 - protein, 6 aa, Homo sapiens
FPQFSY

SEQ ID NO: 47 - protein, 6 aa, Homo sapiens
FLGFSY

SEQ ID NO: 48 - protein, 6 aa, Homo sapiens
FEGFSY

SEQ ID NO: 49 - protein, 6 aa, Homo sapiens
FAGFSY

SEQ ID NO: 50 - protein, 6 aa, Homo sapiens
FEGFSF

SEQ ID NO: 51 - protein, 6 aa, Homo sapiens
FGGFTY

SEQ ID NO: 52 - protein, 6 aa, Homo sapiens
FAGFSF

SEQ ID NO: 53 - protein, 6 aa, Homo sapiens
FEGFEY

SEQ ID NO: 54 - protein, 6 aa, Homo sapiens
FLDFDF

SEQ ID NO: 55 - protein, 6 aa, Homo sapiens
FRDFDY

SEQ ID NO: 56 - protein, 6 aa, Homo sapiens
FLGFTY

SEQ ID NO: 57 - protein, 6 aa, Homo sapiens
FRGFSF

SEQ ID NO: 58 - protein, 6 aa, Homo sapiens
FRDFSF

SEQ ID NO: 59 - protein, 6 aa, Homo sapiens
FRGFSF

SEQ ID NO: 60 - protein, 12 aa, Homo sapiens
SFCGTIEYMAPD

SEQ ID NO: 61 - protein, 12 aa, Homo sapiens
SFCGTIEYMAPE

SEQ ID NO: 62 - protein, 12 aa, Homo sapiens
TLCGTPEYLAPE

SEQ ID NO: 63 - protein, 12 aa, Homo sapiens
SFVGTAQYVSPE

SEQ ID NO: 64 - protein, 6 aa, Homo sapiens
FQGYSF

SEQ ID NO: 65 - protein, 4 aa, Homo sapiens
FSEF

SEQ ID NO: 66 - protein, 6 aa, Artificial Sequence
  VARIANT (2) or (3): Variable amino acid
FXXFTY

SEQ ID NO: 67 - protein, 6 aa, Artificial Sequence
  VARIANT (2) or (3): Variable amino acid
FXXFEY

SEQ ID NO: 68 - protein, 6 aa, Artificial Sequence
  VARIANT (2) or (3): Variable amino acid
FXXFAY